SEQUENCE LISTING (7 sequences)

SEQ ID NO: 1 - DNA, 20 bp, Mus musculus
gagttcatgcgcttcaaggt

SEQ ID NO: 2 - DNA, 20 bp, Mus musculus
cccatggtcttcttctgcat

SEQ ID NO: 3 - DNA, 20 bp, Mus musculus
cgccatggatcctgatgatg

SEQ ID NO: 4 - DNA, 26 bp, Mus musculus
ccatggcttcacaaagatcgcctgac

SEQ ID NO: 5 - DNA, 19 bp, Discosoma
agcgcgtgatgaacttcga

SEQ ID NO: 6 - DNA, 24 bp, Discosoma
gccgatgaacttcaccttgtagat

SEQ ID NO: 7 - DNA, 15 bp, Discosoma
acccaggactcctcc

(19) United States
(12) Patent Application Publication
(10) Pub. No.: US 2014/0142035 A1
(43) Pub. Date: May 22, 2014

(54) METHODS AND COMPOSITIONS FOR MODULATING INSULIN REGULATION

(71) Applicant: The Washington University, St. Louis, MO (US)

(72) Inventors: Burton M. Wice, St. Louis, MO (US); Kenneth S. Polonsky, St. Louis, MO (US)

(21) Appl. No.: 14/065,381

(22) Filed: Oct. 28, 2013

Related U.S. Application Data
(62) Division of application No. 12/868,307, filed on Aug. 25, 2010, now abandoned.

(51) Int. Cl. A61K 38/22

(52) U.S. Cl. CPC A61K 38/22
USPC 514/6.7; 514/9.7

(57) ABSTRACT

The present invention provides a combination of compounds capable of modulating xenin activity and, insulin secretion and weight gain. The invention also provides methods for modulating GIP activity and insulin secretion in a subject, by modulating xenin activity in the subject.

### CROSS REFERENCE TO RELATED APPLICATIONS

The application claims the priority of U.S. patent application Ser. No. 12/868,307 filed Aug. 25, 2010, which claims the priority of International Application No. PCT/US2009/034965, filed Feb. 24, 2009, which claims the priority of U.S. Provisional Application Ser. No. 61/031,285, each of which is hereby incorporated by reference in its entirety.

### GOVERNMENTAL RIGHTS

This invention was made with government support under DK-31842 and P30 DK-56341 awarded by the National Institutes of Health. The government has certain rights in the invention.

### FIELD OF THE INVENTION

The present invention generally provides for compositions and methods of modulating insulin secretion and weight gain.

### BACKGROUND OF THE INVENTION

The entero-insulin axis is a physiological system that comprises peptides secreted from the gastro-intestinal tract that play an important role in regulating insulin secretion from the pancreatic islet beta cell. To date attention has been focused on two intestinal peptides, glucagon like peptide 1 (GLP-1) and glucose-dependent insulinotropic polypeptide (GIP). Both of these hormones are released into the blood immediately after ingestion of a meal and potentiate glucose-stimulated insulin release. The increase in insulin secretion precipitated by these so called incretin peptides has been termed the incretin effect. Importantly, incretin-mediated potentiation of glucose-stimulated insulin release occurs only in the presence of elevated blood glucose. This critical property of incretins prevents continued insulin release and subsequent hypoglycemia once blood glucose levels return to normal.

An increase in the activity of the circulating incretin GLP-1 has significant therapeutic benefit in patients with type 2 diabetes. Two drugs that accomplish this goal have recently been introduced into the market with substantial success. Exenatide is a GLP-1 analogue that increases insulin secretion leading to substantial improvements in glucose control in patients with type 2 diabetes. Sitagliptin inhibits the enzyme dipeptidyl peptidase IV (DPP IV) responsible for GLP-1 breakdown in the circulation and increases circulating levels of endogenous GLP-1 by reducing its metabolism.

The other major incretin hormone, GIP, has been reported as ineffective in persons with type 2 diabetes. Therefore, potential therapeutics based on GIP has not been pursued. Given the rise in the incidence of type 2 diabetes and obesity, there is a need in the art for additional therapeutics that target the entero-insulin axis.

### SUMMARY OF THE INVENTION

One aspect of the invention encompasses a combination comprising a compound capable of modulating xenin activity and at least one compound selected from the group consisting of compounds capable of modulating insulin secretion and compounds capable of modulating weight gain.

Another aspect of the invention encompasses a method for modulating GIP activity in a subject. The method comprises administering a composition to the subject, wherein the composition modulates the xenin activity in the subject.

Yet another aspect of the invention encompasses a method for modulating insulin secretion in a subject. The method comprises administering to the subject a compound capable of modulating xenin activity in the subject.

Other aspects and iterations of the invention are described more thoroughly below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

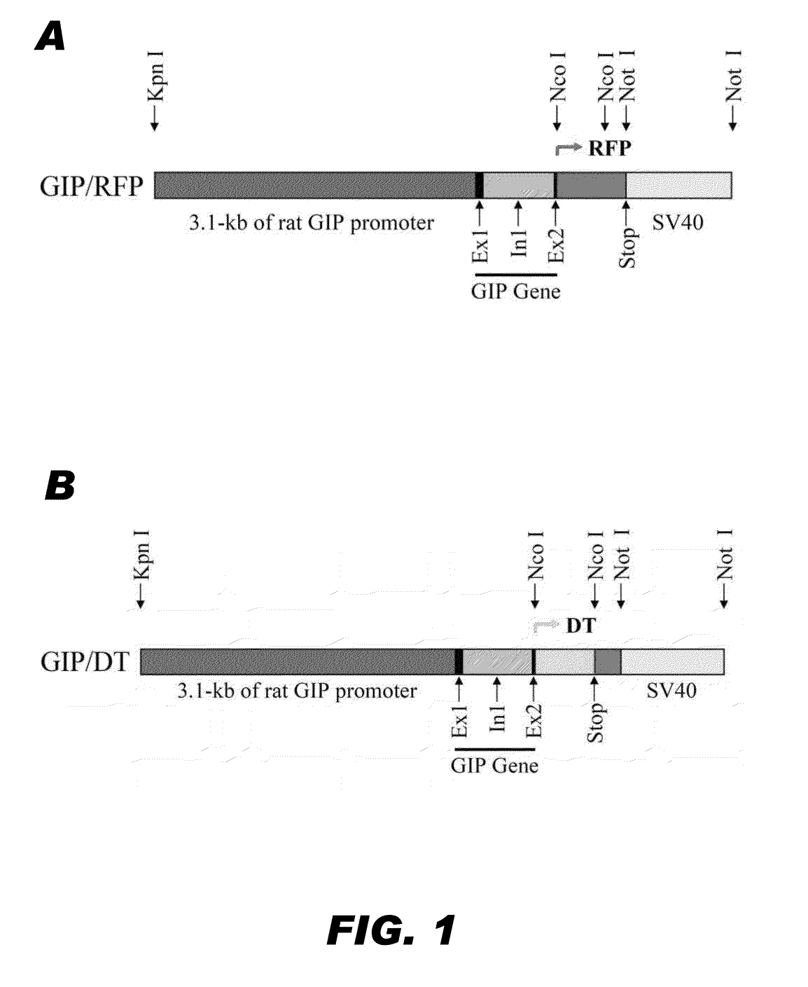

FIG. 1 depicts an illustration of two transgenic constructs. (a) A fragment containing the GIP promoter through the Nco I site within the initiator methionine in exon 2 of the GIP structural gene was isolated and fused in frame to the initiator methionine encoding RFP. A second intron, a splice site, and polyadenylation signals are present in the 3′-UTR from SV40 Large T antigen. The resulting GIP/RFP construct encodes a chimeric mRNA transcript but not a chimeric RFP protein. Colors represent the GIP promoter (dark blue); exonic (Ex) sequences from the GIP gene (black), intron 1 (In 1) from the GIP gene (gray), RFP cDNA (red), the SV40 3′-UTR (light blue). (b) To generate the GIP/DT construct, an Nco I fragment from the RFP cDNA was replaced with an Nco I fragment encoding the DT cDNA (green). The attenuated diphtheria toxin A chain without any additional amino acids was generated from the chimeric transcript.

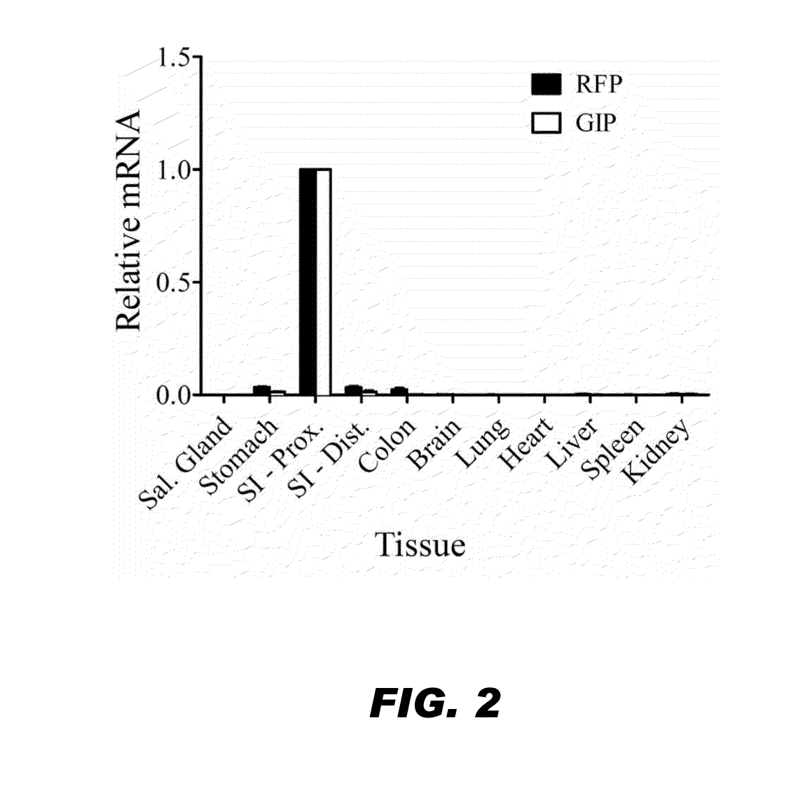

FIG. 2 depicts a graph showing that regulatory sequences from the rat GIP promoter and structural gene confer proper transgene expression in vivo. RNA was isolated from the indicated tissue from GIP/RFP mice and assayed for GIP and RFP using real time PCR. Values are relative to those in the most proximal 2-cm of small intestine (SI-Prox). Note that like the endogenous GIP gene, RFP is expressed at high levels in the proximal small intestine and at much lower levels in the stomach and the most distal 2-cm of the small intestine (SI-Dist). Expression in the first 2-cm of colon is 50-fold less than that in the proximal small intestine. GIP and RFP transcripts were not expressed at detectable levels in the submandibular salivary (Sal) gland.

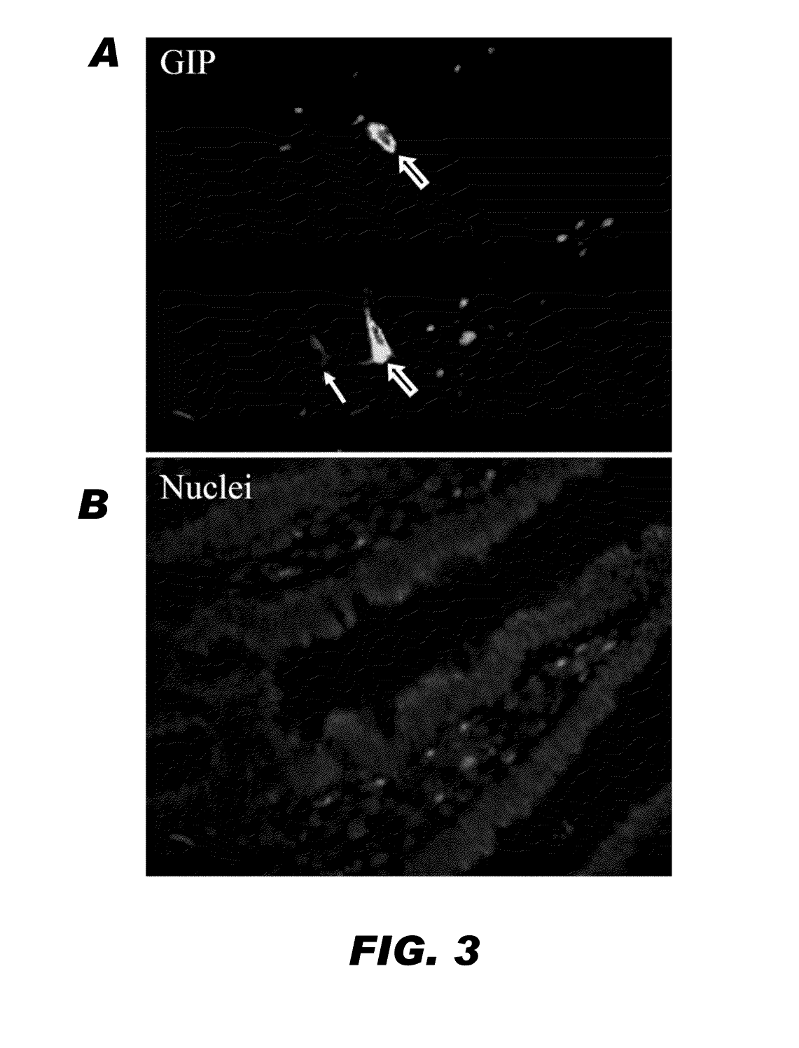

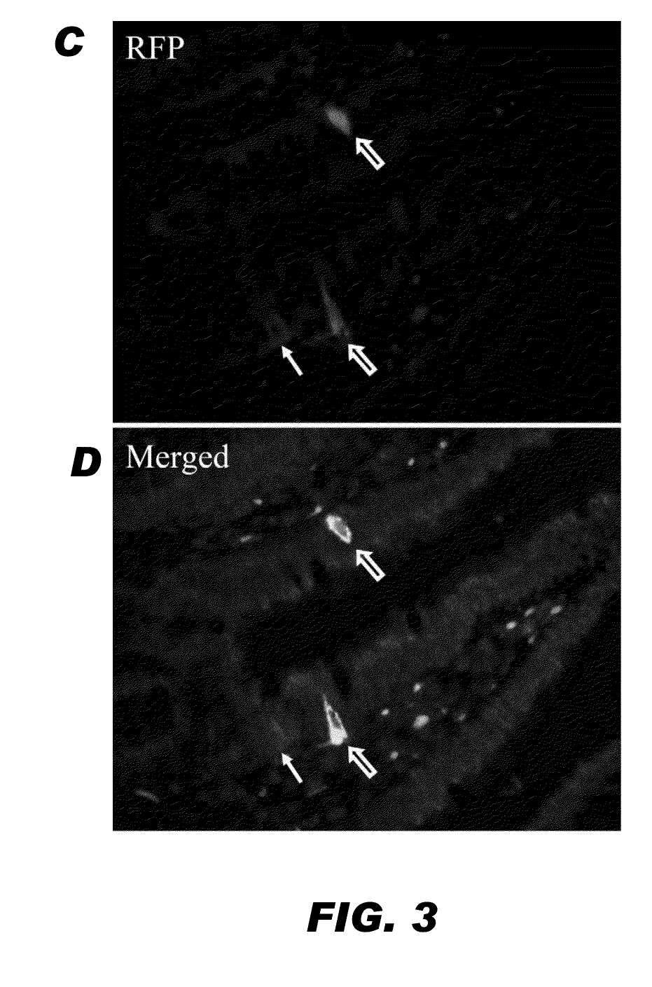

FIG. 3 depicts micrographs showing that GIP/RFP transgene expression is confined to GIP-producing cells in the intestinal epithelium. A single paraffin embedded section from a GIP/RFP mouse intestine was stained with antibodies to RFP (red) (c) plus GIP (green) (a). Nuclei were counterstained blue (b). Each fluorescent dye was photographed as a single color and then overlaid in Adobe Photoshop to generate a merged image (d) Note that high levels of GIP and RFP are expressed in the same scattered, rare cells in the intestinal epithelium (open arrows). Low levels of both RFP and GIP can also be seen in a third cell (solid arrow). RFP staining was not observed in sections prepared from the intestines from wild type mice.

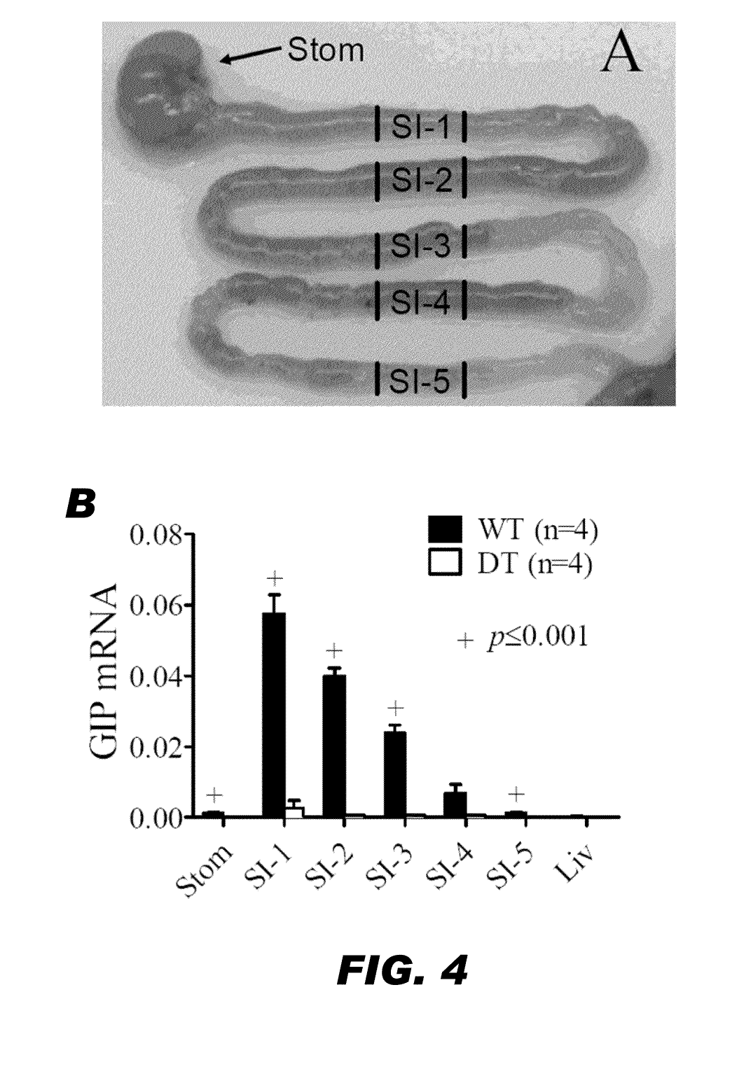

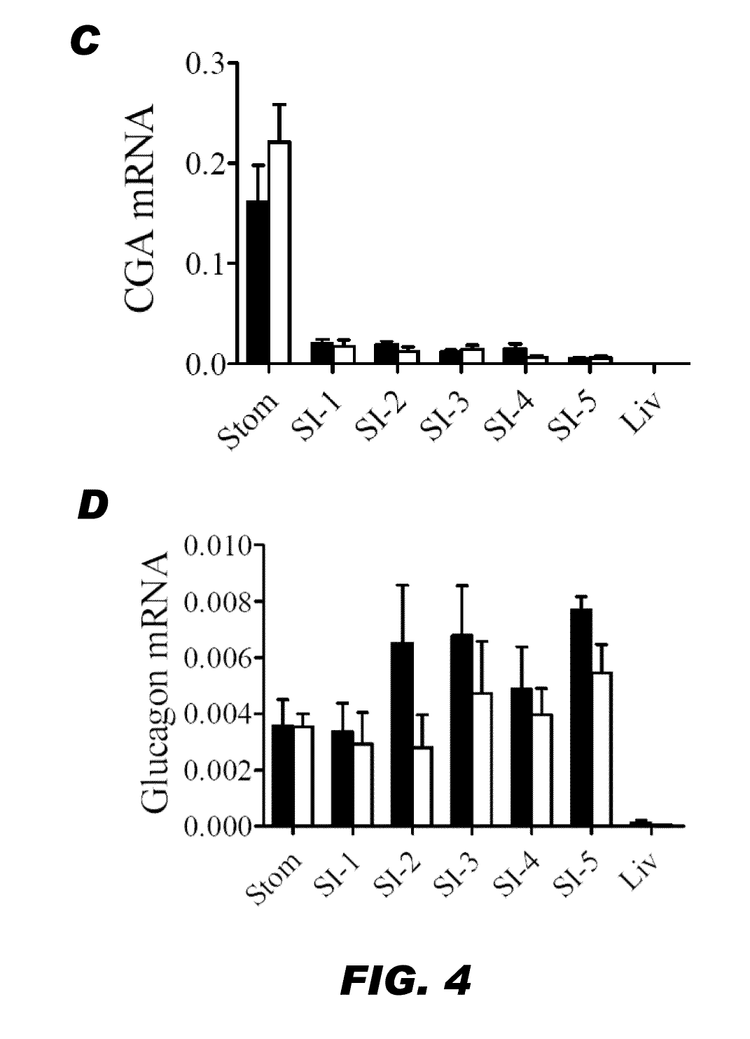

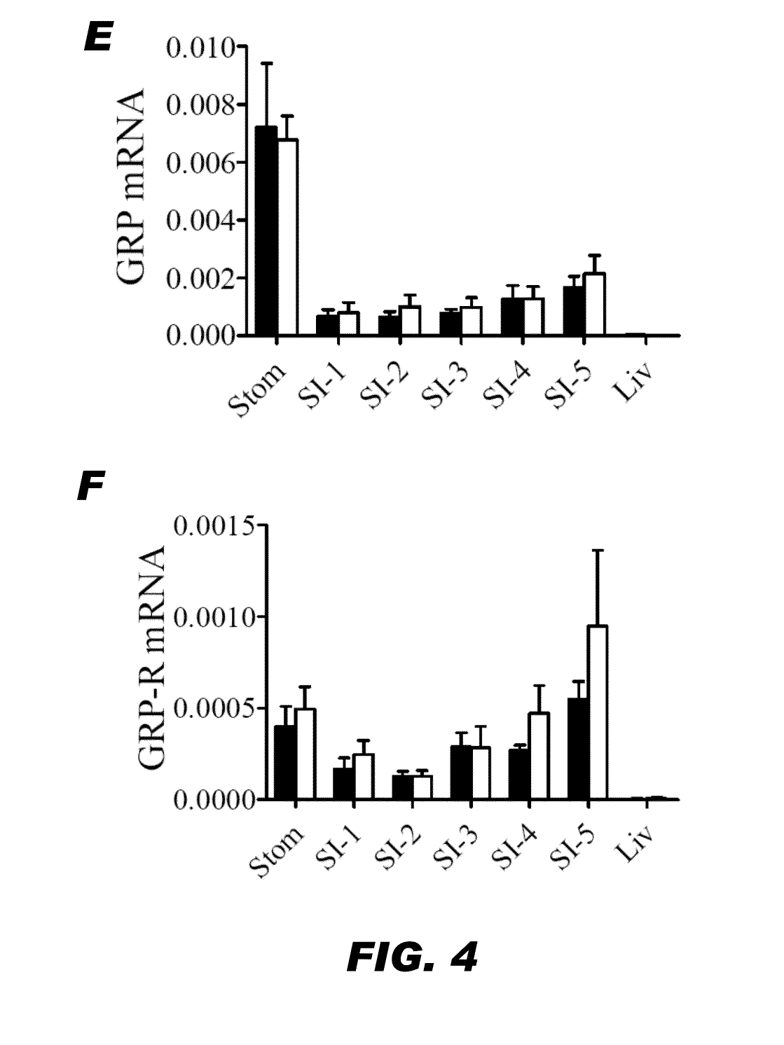

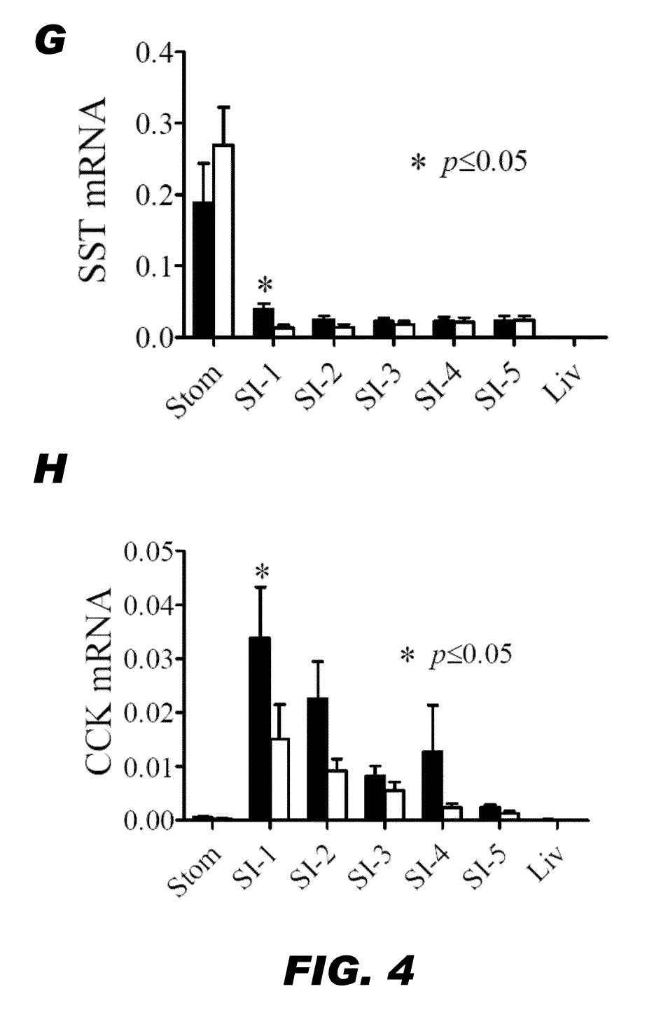

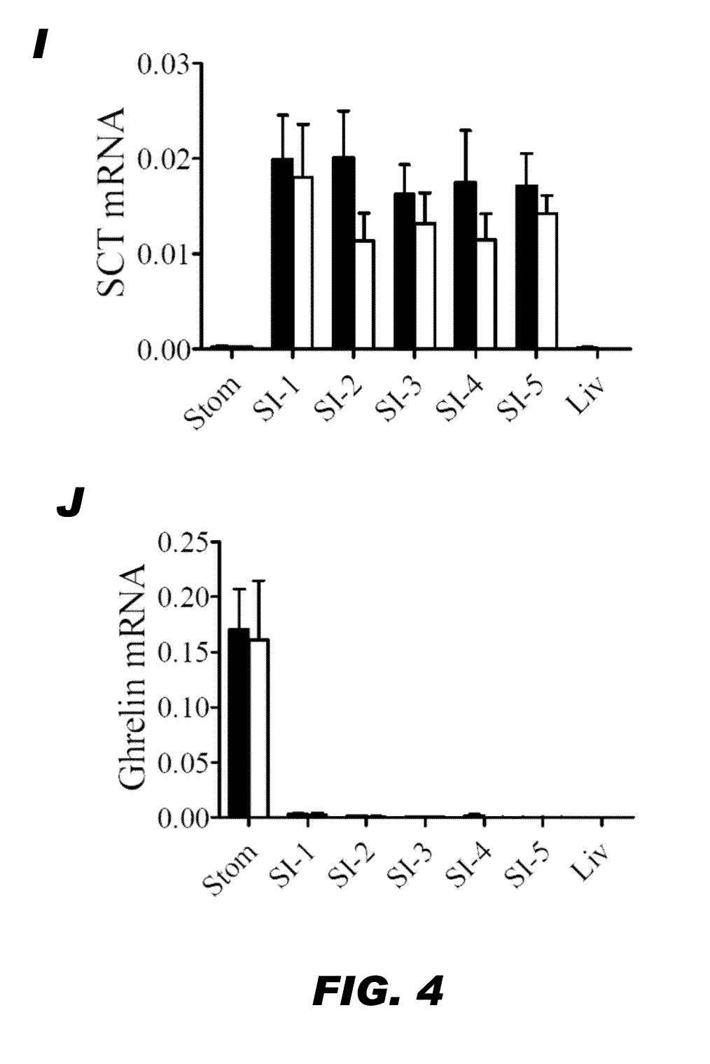

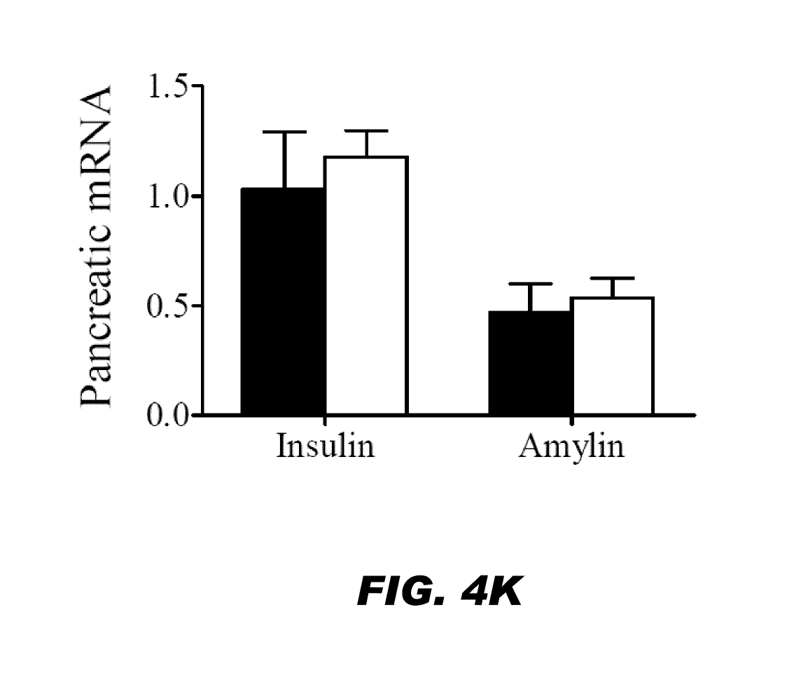

FIG. 4 depicts a series of graphs showing that transcripts encoding GIP, but not other enteroendocrine (EE) cell products, are greatly reduced in GIP/DT mice. RNA was isolated from the stomach, from the mid-portion of sequential segments of the small intestine [SI-1 to SI-5 (see Panel A)], and from the pancreas. Liver served as a negative control. Real time PCR was used to quantify the mRNA levels for the indicated transcript. Note that GIP mRNA levels (b) are nearly absent in the GIP/DT (DT) mice whereas transcripts that encode all other products are essentially normal (c)-(j). Also note that transcripts encoding insulin and amylin, produced by pancreatic islet b-cells, are normal in the GIP/DT mice (k). Results represent the average from 4 wild type (WT) and 4 GIP/DT mice.

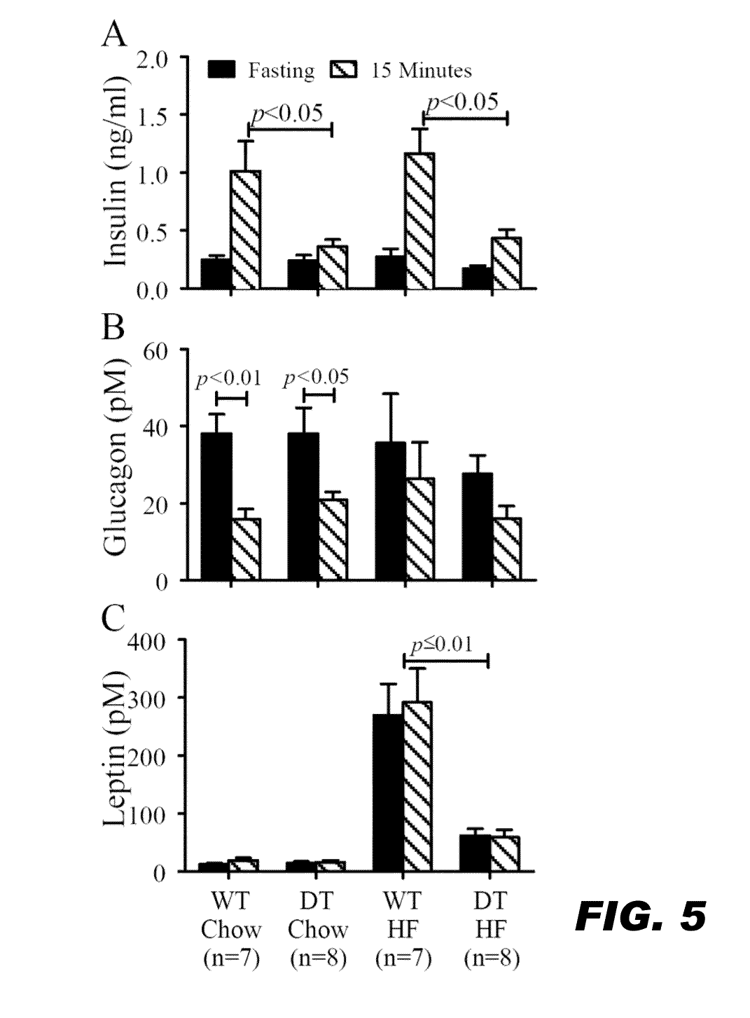

FIG. 5 depicts three graphs showing that the incretin effect is absent in mice lacking GIP-producing cells. Wild type (WT) and GIP/DT (DT) mice fed standard chow (Chow) or high fat food (HF) for 15 weeks were fasted for 16 h. Plasma hormone levels were determined in plasma prepared from blood that was collected from the same animals before and 15-minutes after administration of glucose (3 mg per g body weight) by intragastric gavage. Panel A: Note that oral glucose-stimulated insulin release is essentially absent in the GIP/DT mice fed standard chow but is partially restored following high fat feeding. Panel B: Note that glucagon levels and responses are similar in wild type and GIP/DT mice. Panel C: Note that GIP/DT mice fed high fat diet produce much less leptin than wild type animals. It should be noted that leptin levels reflect adipose mass and are not increased by acute ingestion of nutrients. Thus, the 0- and 15-minutes values represent duplicates.

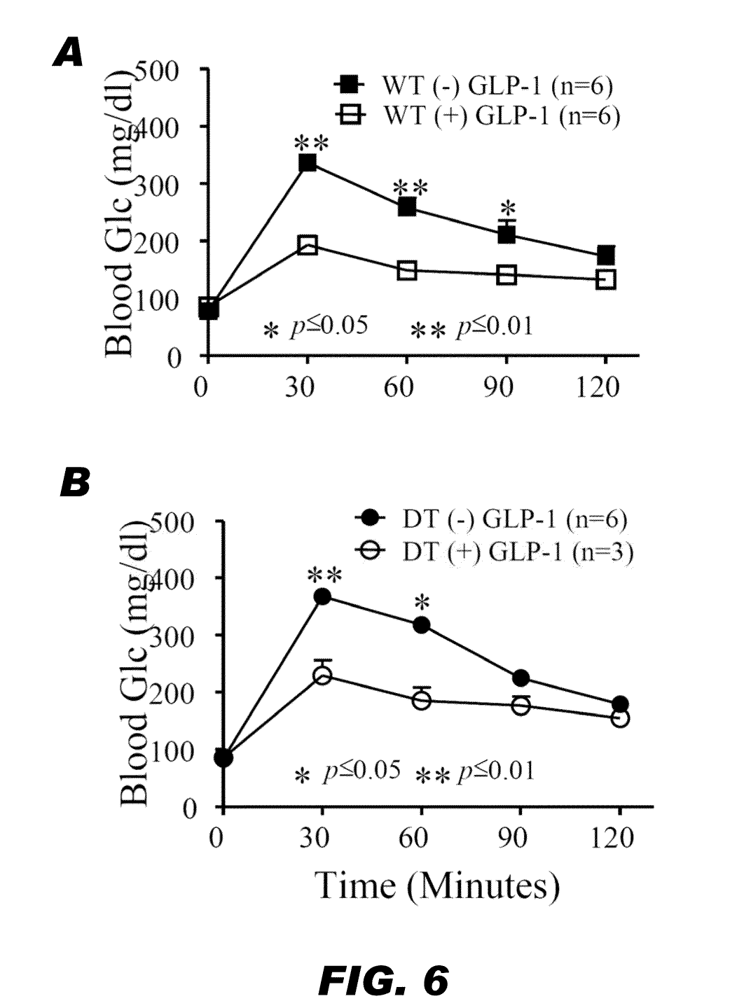

FIG. 6 depicts two graphs showing that exogenous GLP-1 potentiates intraperitoneal glucose-stimulated insulin release in mice lacking GIP-producing cells. Wild type (WT) (a) and GIP/DT (DT) (b) mice were fasted overnight. Blood glucose levels were measured before (0 minutes) and at the indicated time after intraperitoneal administration of glucose (1 mg per g body weight) with (+) or without (−) 1 nmole synthetic GLP-1(7-36). Note that GLP-1 potentiated glucose clearance to similar extents in wild type and GIP/DT mice.

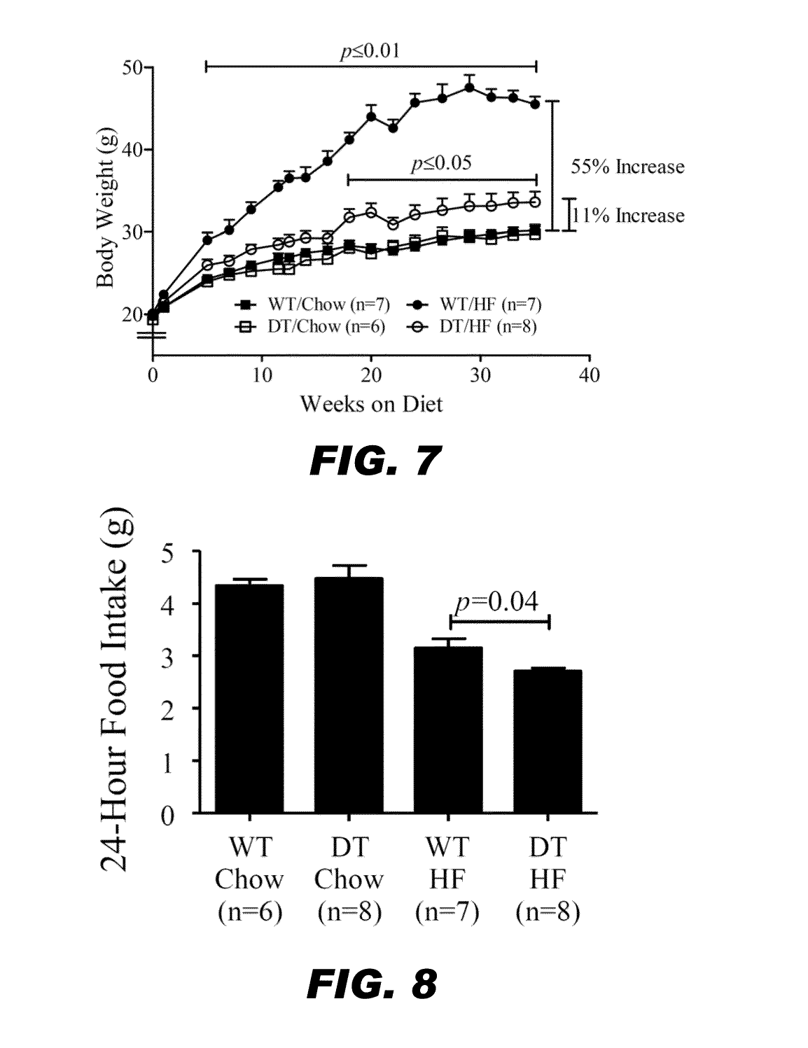

FIG. 7 depicts a graph showing that GIP/DT mice resist development of high fat diet-induced obesity. At 8 weeks of age, groups of mice were either switched to the high fat (HF) diet or maintained on standard chow (Chow). Body weights were determined at the indicated time after commencing the high fat diet. Note that wild type (WT) mice fed a high fat diet weigh 55% more than those maintained on standard chow. Conversely, high fat feeding promoted only an 11% gain in body weight of the GIP/DT animals.

FIG. 8 depicts a graph showing that high fat food intake is reduced in mice lacking GIP-producing cells. Food intake per mouse was measured in individually housed, well-acclimated wild type (WT) and GIP/DT (DT) mice fed standard chow or a HF diet for 21-weeks. Values represent the amount of food consumed per 24 h and are the average from 5 consecutive days. Note that on the HF diet, the transgenic animals eat ˜16% less HF food per day.

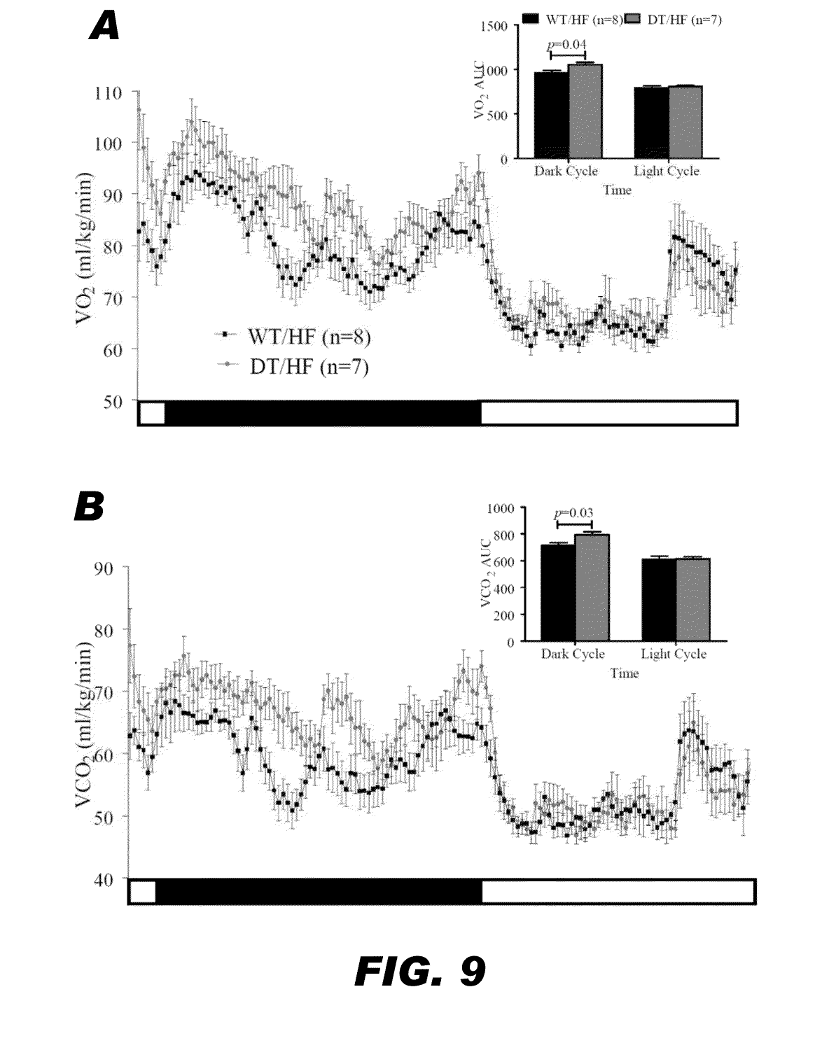

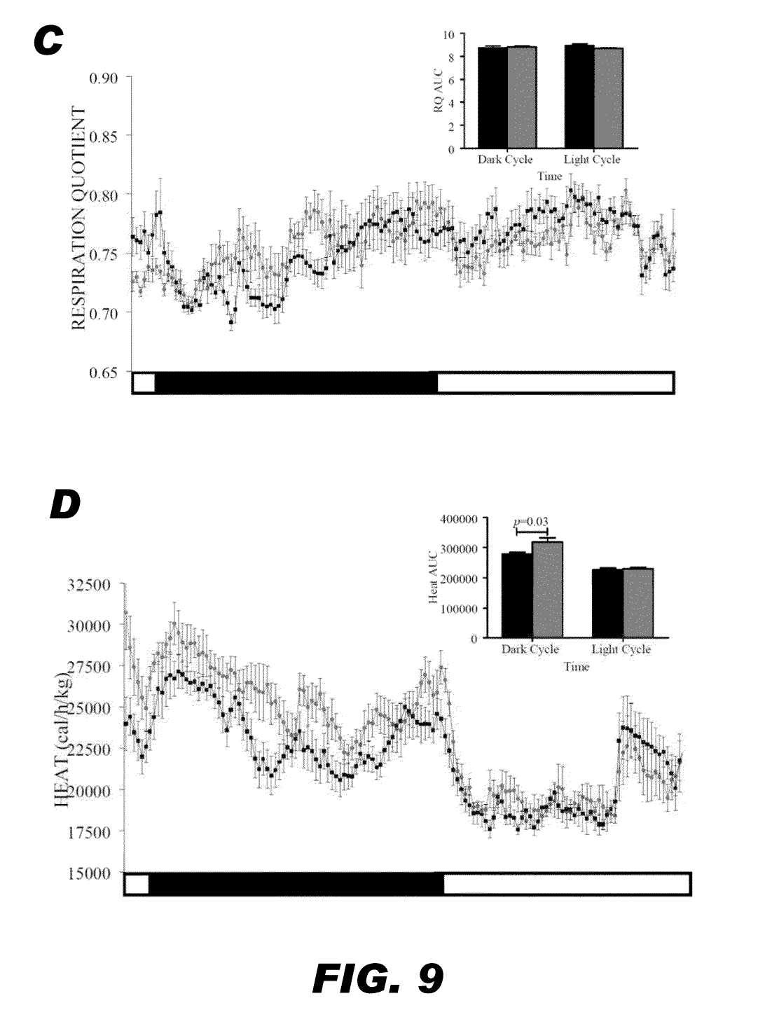

FIG. 9 depicts four graphs showing that energy expenditure is increased in mice lacking GIP-producing cells fed a high fat diet. Following 13 weeks of high fat feeding, wild type (black) and GIP/DT (red) mice were placed in the PhysioScan chamber and energy expenditure was recorded for 24 h. Black and white bars represent dark and light cycles, respectively. Insets show the area under the curve (AUC) for oxygen consumption (VO2) (a), carbon dioxide production (VCO2) (b), respiration quotient (c), and heat output (d). Note that energy expenditure is increased in GIP/DT mice during the dark cycle.

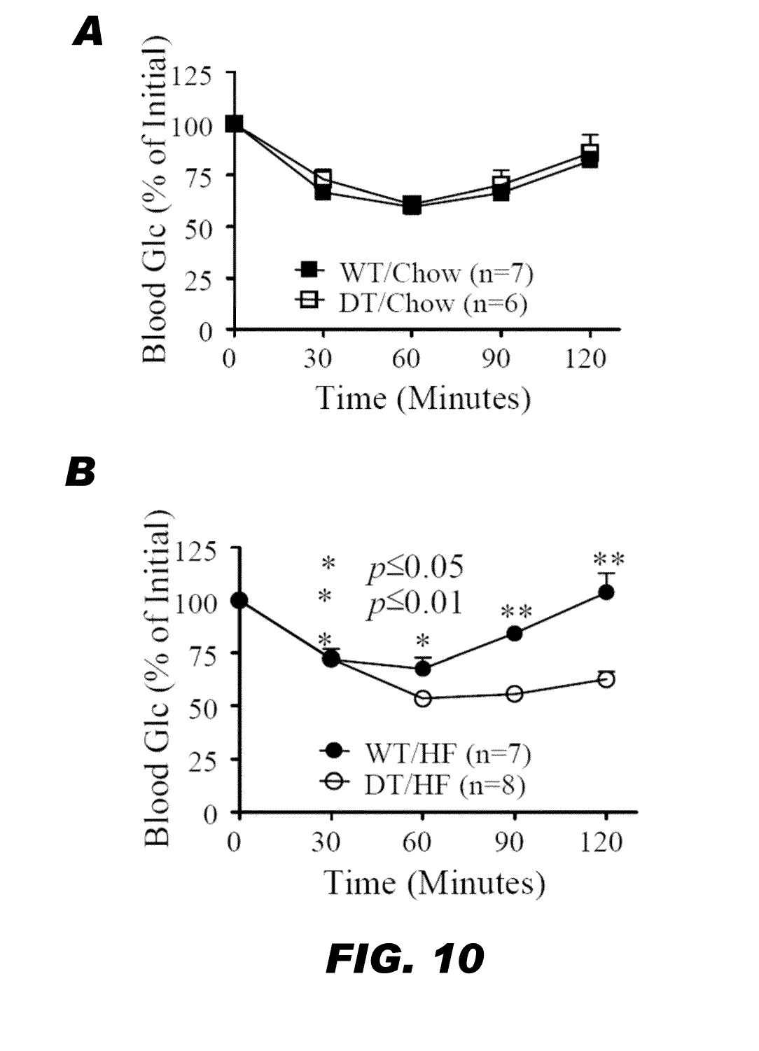

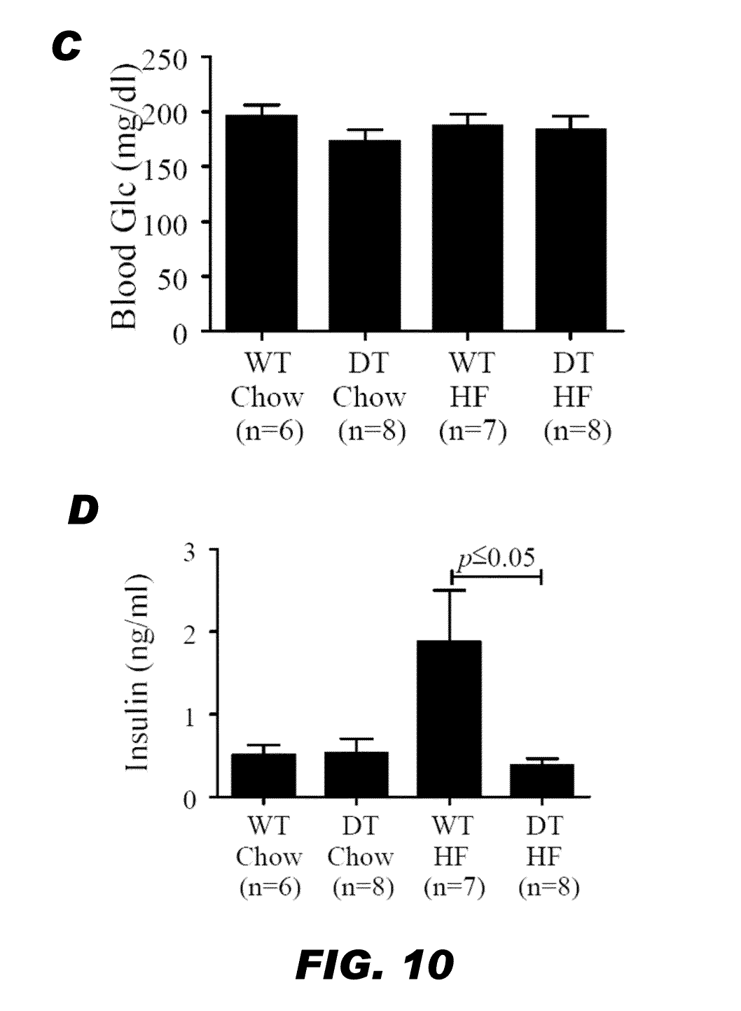

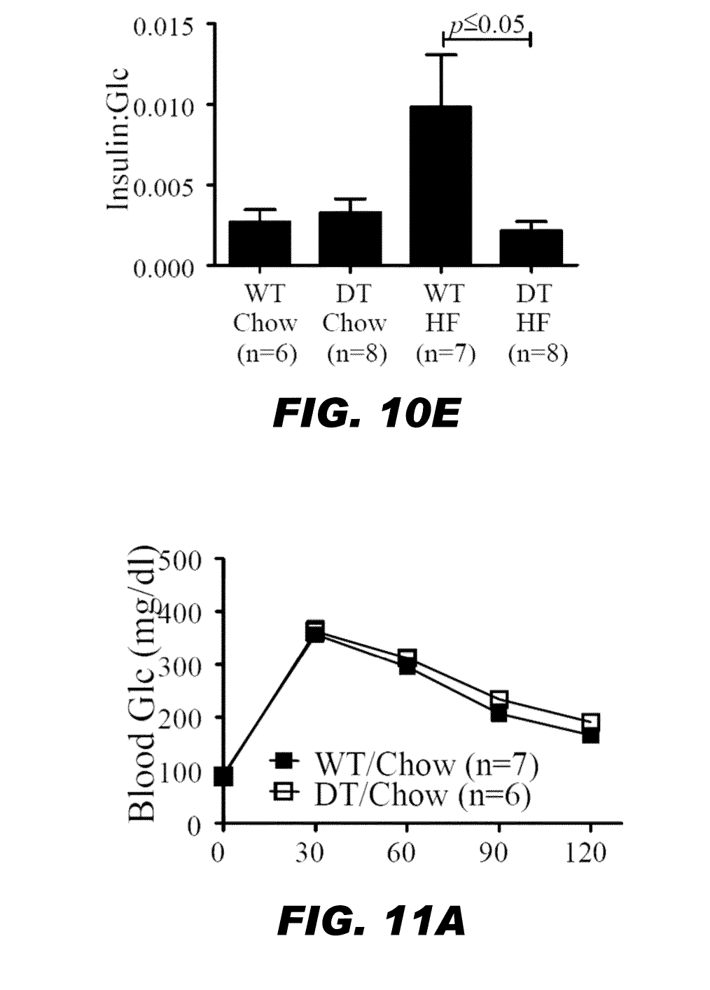

FIG. 10 depicts a series of graphs showing that insulin sensitivity is improved in GIP/DT mice fed a high fat diet. Panels A and B, Insulin tolerance tests (ITT): Wild type (WT) and GIP/DT (DT) mice fed standard chow (Chow; Panel A) or high fat food (HF; Panel B) for 33 weeks were fasted for 5 h and then administered human insulin by intraperitoneal injection (0.5 units/kg body weight). Blood glucose levels were determined before (0 minutes) and at the indicated time following administration of insulin. Note that glucose clearance rates from blood are identical in wild type and GIP/DT mice fed standard chow whereas insulin sensitivity is greatest in GIP/DT mice fed high fat food. Panels C to E; Insulin to glucose ratios: Mice were maintained on standard chow or high fat food for 27-weeks. Blood was then collected from non-fasted wild type and GIP/DT mice between 10 AM and noon. (Panel C). Blood glucose and plasma insulin levels are shown in panels C and D, respectively. The insulin to glucose ratio (Panel E) was calculated using glucose and insulin values from individual mice. Note that insulin levels and insulin to glucose ratios are elevated only in wild type mice fed a high fat diet indicating wild type, but not GIP/DT mice, are insulin resistant.

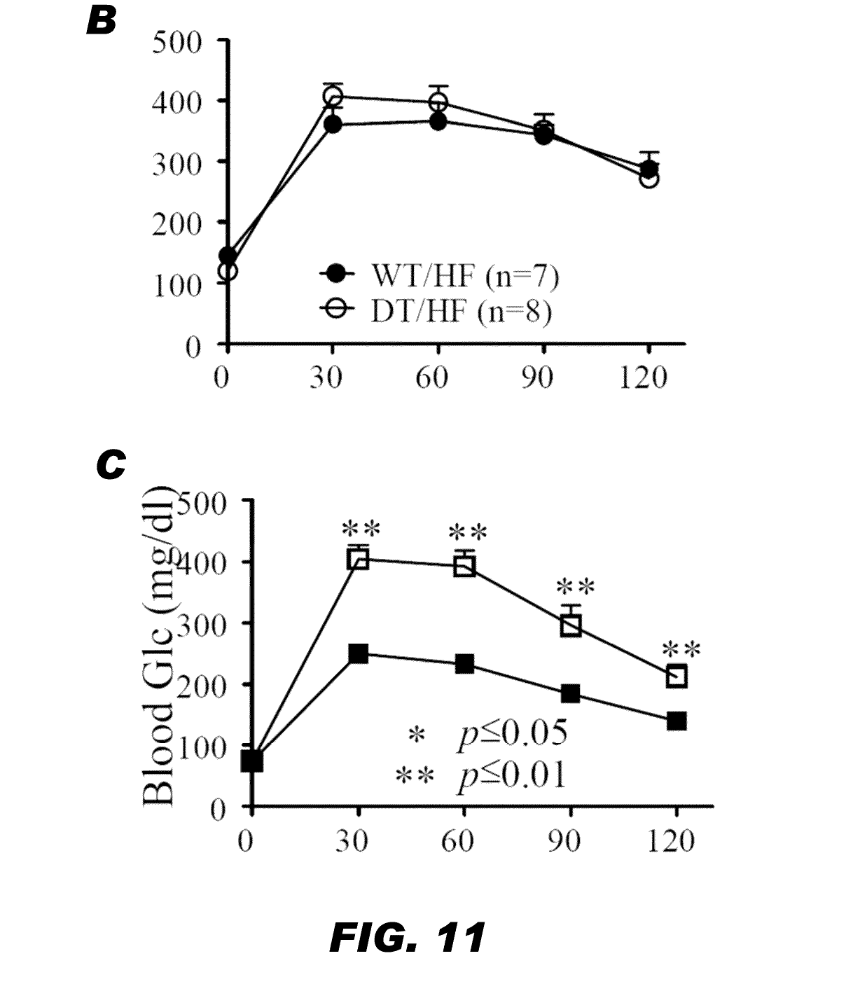

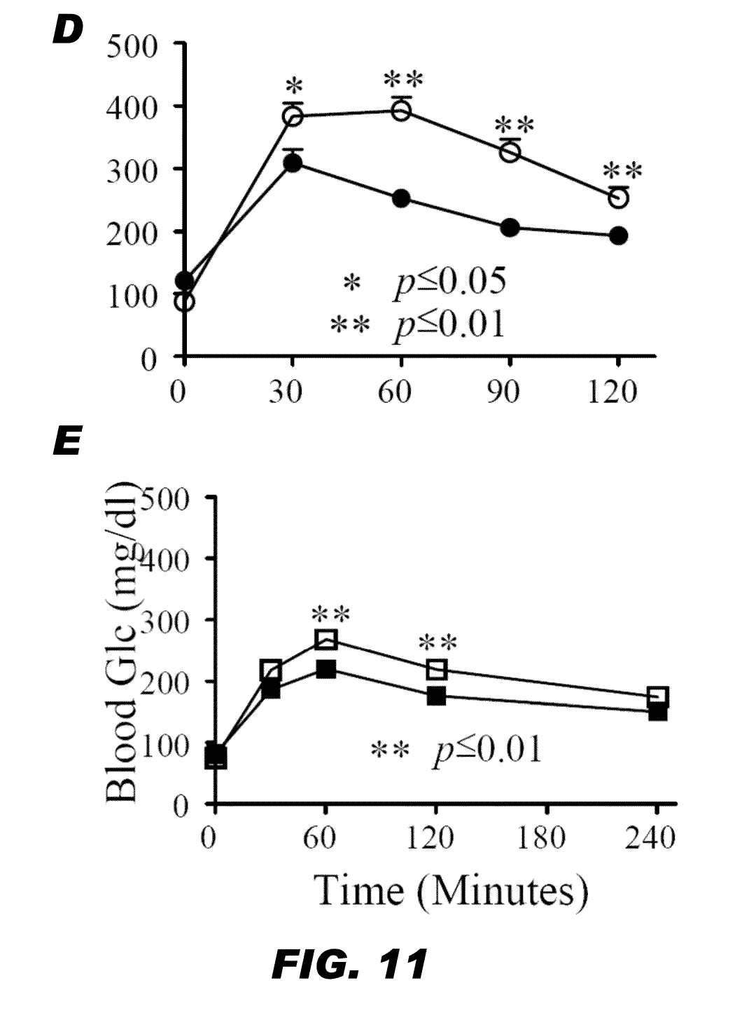

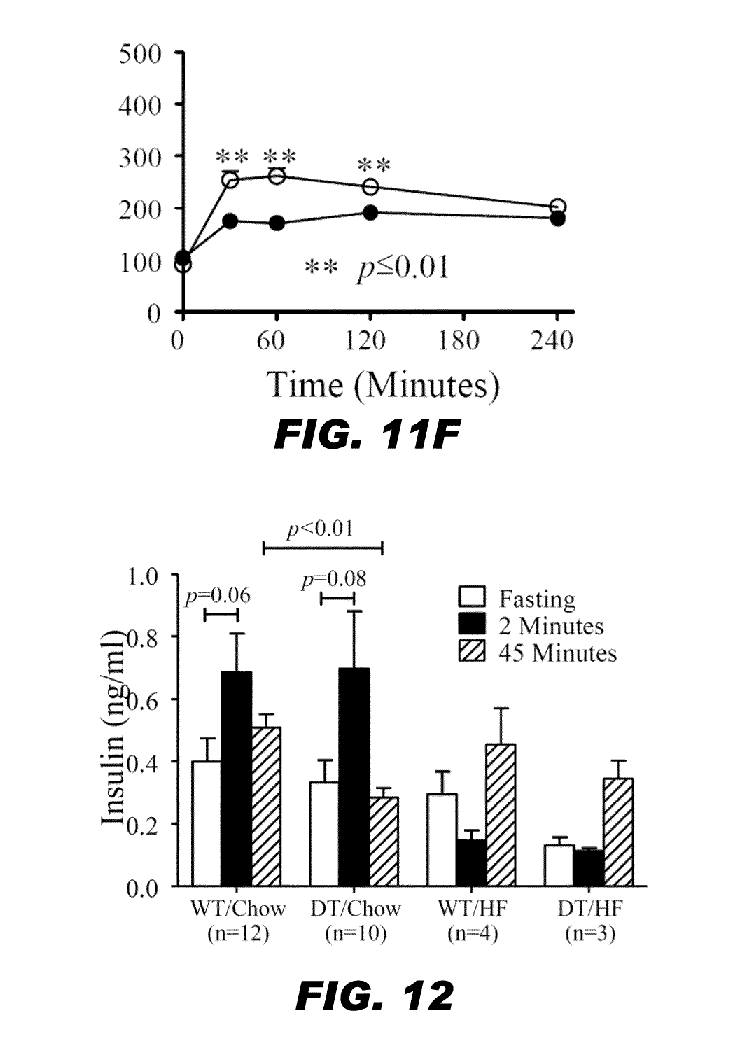

FIG. 11 depicts a series of graphs showing that glucose homeostasis is similar in wild type (WT) and GIP/DT (DT) mice. Mice were fasted for 16 h. Blood glucose levels were determined before (time 0) and at the indicated time after animals were given intraperitoneal glucose (1 mg per g body weight; IPGTT; Panels A and B), oral glucose (3 mg per g body weight; OGTT; Panels C and D), or free access to standard chow (Chow) or high fat (HF) food (FTT; Panels E and F). Panels A and B. Note that on standard chow, the GIP/DT mice exhibited normal clearance of glucose from blood following administration of intraperitoneal glucose. Conversely, high fat feeding resulted in a similarly reduced rate of glucose clearance in both wild type and GIP/DT mice compared to parallel groups on a standard chow diet. Panels C and D. Note that GIP/DT mice fed either standard chow or high fat food exhibit impaired oral glucose tolerance due to the lack of an incretin effect. However, high fat feeding worsened oral glucose tolerance only in the wild type mice. Panels E and F. Note that in contrast to administration of oral glucose, normal food intake results in only a modest increase in blood glucose levels. IPGTT, OGTT, and FTT were conducted following 30, 31, and 20 weeks, respectively, on a high fat diet.

FIG. 12 depicts a graph showing that intraperitoneal glucose stimulated insulin release is normal in mice lacking GIP-producing cells. Wild type (WT) and GIP/DT (DT) mice were fasted for 16 h. Blood was collected at the indicated time before (Fasting) or after intraperitoneal injection of glucose (1 mg per g body weight).

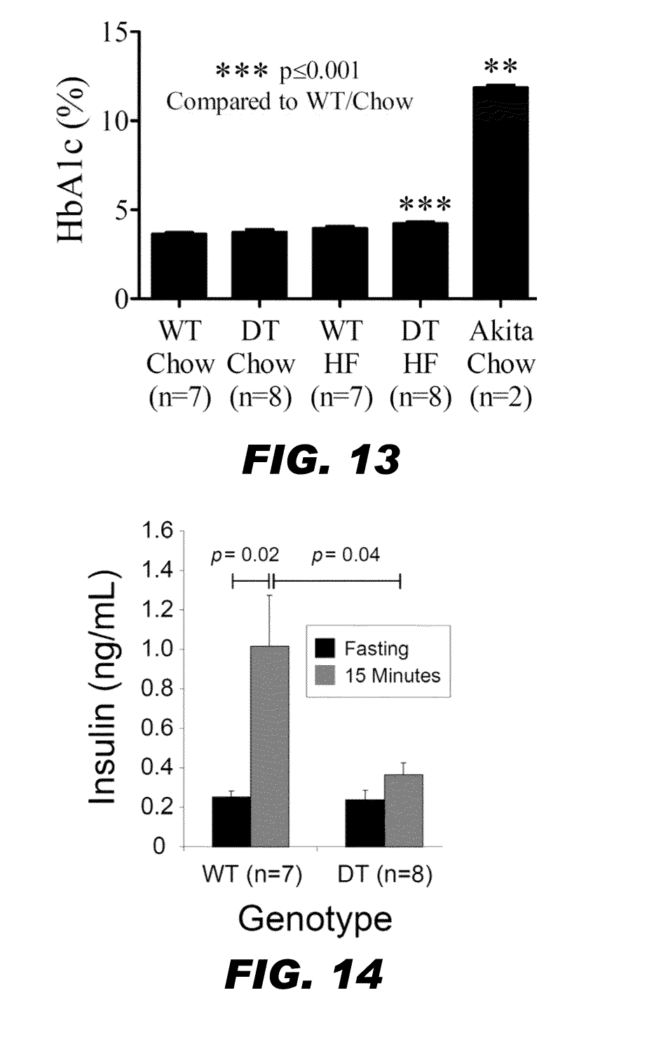

FIG. 13 depicts a graph showing that long-term glucose homeostasis is not perturbed in GIP/DT mice. HbA1c levels were determined in blood collected from wild type (WT) and GIP/DT (DT) mice that were fed standard chow (Chow) or high fat (HF) food for 36 weeks. Diabetic Akita mice on a C57BL/6J background served as positive control to confirm the validity of the assay. Note that HbA1c levels are similar in wild type and GIP/DT mice fed standard chow and are elevated only 15% in the GIP/DT mice fed the high fat diet.

FIG. 14 depicts a graph showing that the incretin response is nearly absent in mice lacking GIP-producing cells. Novel transgenic GIP/DT (DT) mice were generated that express an attenuated diphtheria transgene using regulatory elements from the rat GIP promoter/gene. The DT mice lack nearly all GIP and any other product produced by GIP-producing cells. Wild type (WT) and DT mice fed standard chow were fasted for 16 h. Plasma insulin levels were determined before and 15-minutes after administration of glucose (3 mg per g body weight) by intragastric gavage. Note that oral glucose-stimulated insulin release is essentially absent in the DT mice.

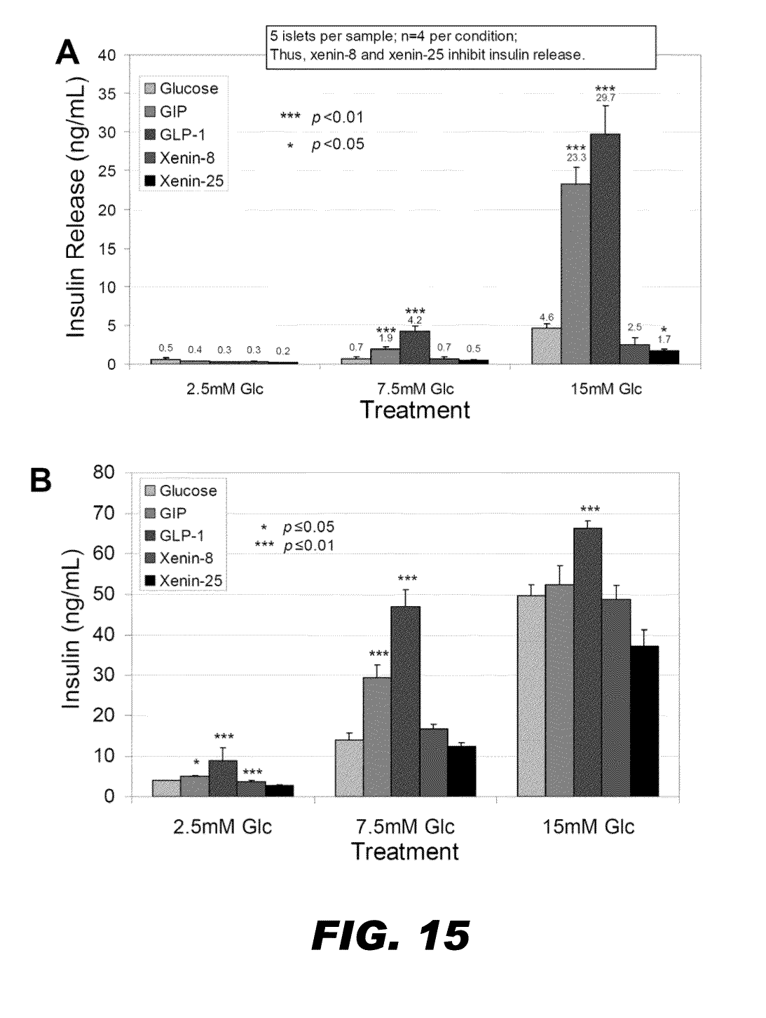

FIG. 15 depicts two graphs showing that xenin does not exhibit classical incretin activity in primary mouse islets or MIN6 cells. Primary mouse islets (panel A) or MIN6 cells (panel B) were incubated for 1-h in insulin assay buffer (KRB) containing 2.5 mM glucose. Islets or cells were then switched to KRB containing the indicated concentration of glucose with or without 100 nM GIP, GLP-1, Xenin-8, or Xenin-25. Sixty-minutes later, assay buffer was collected and the amount of insulin released determined by RIA. Note that GIP and GLP-1, but not Xenin-8 or -25, potentiate glucose-stimulated insulin release.

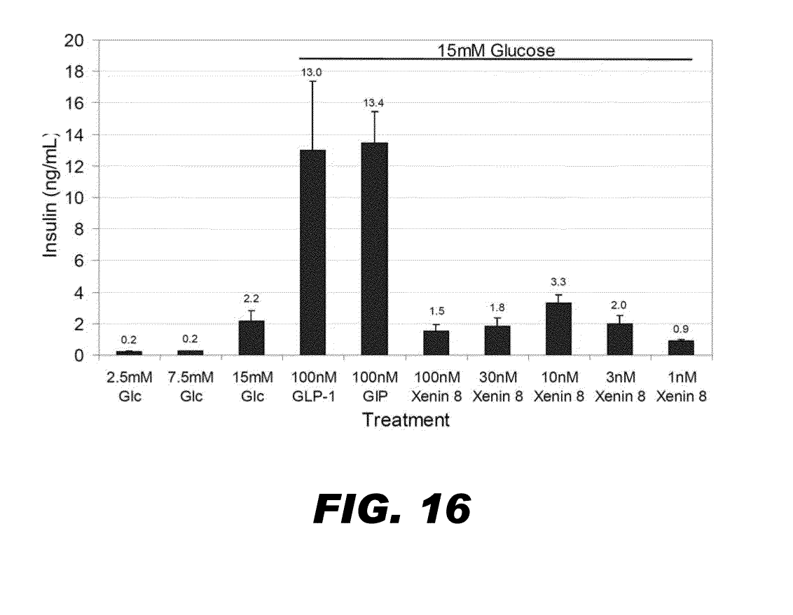

FIG. 16 depicts a graph showing that xenin does not exhibit classical incretin activity in primary mouse islets. Primary mouse islets were incubated for 1-h in insulin assay buffer (KRB) containing 2.5 mM glucose. Islets were then switched to KRB containing the indicated concentration of glucose with or without 100 nM Xenin-8. Sixty-minutes later, assay buffer was collected and the amount of insulin released determined by RIA. Note that GIP and GLP-1, but not Xenin-8, potentiate glucose-stimulated insulin release.

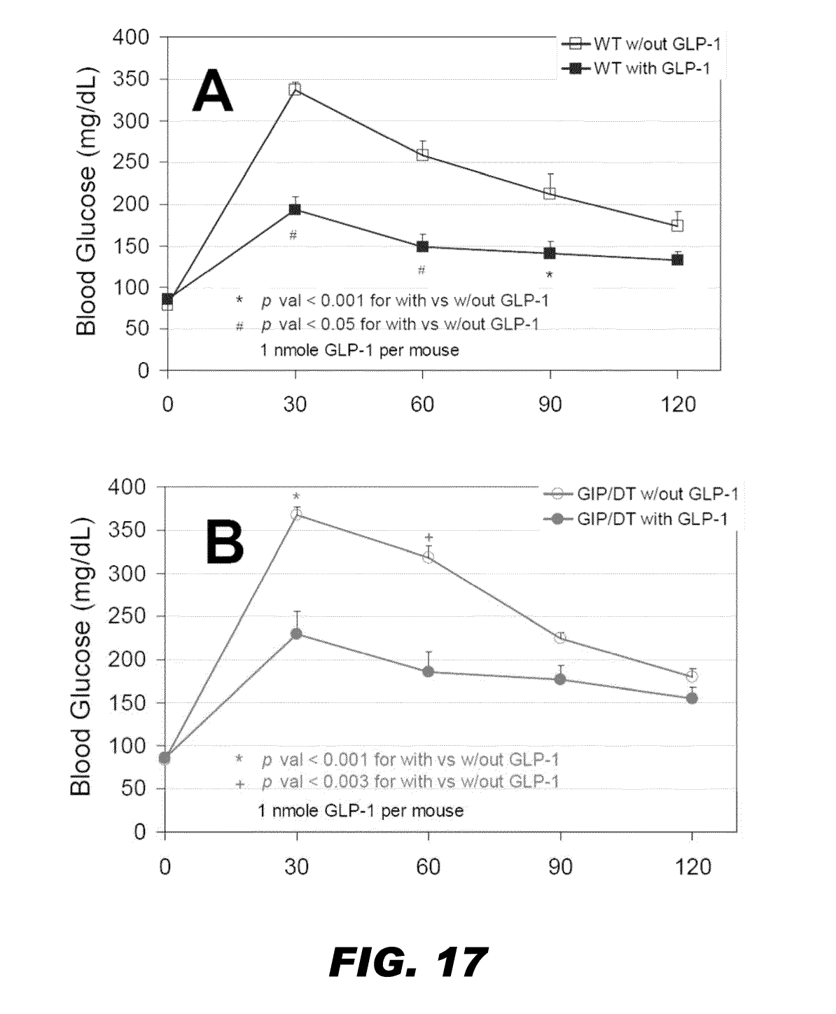

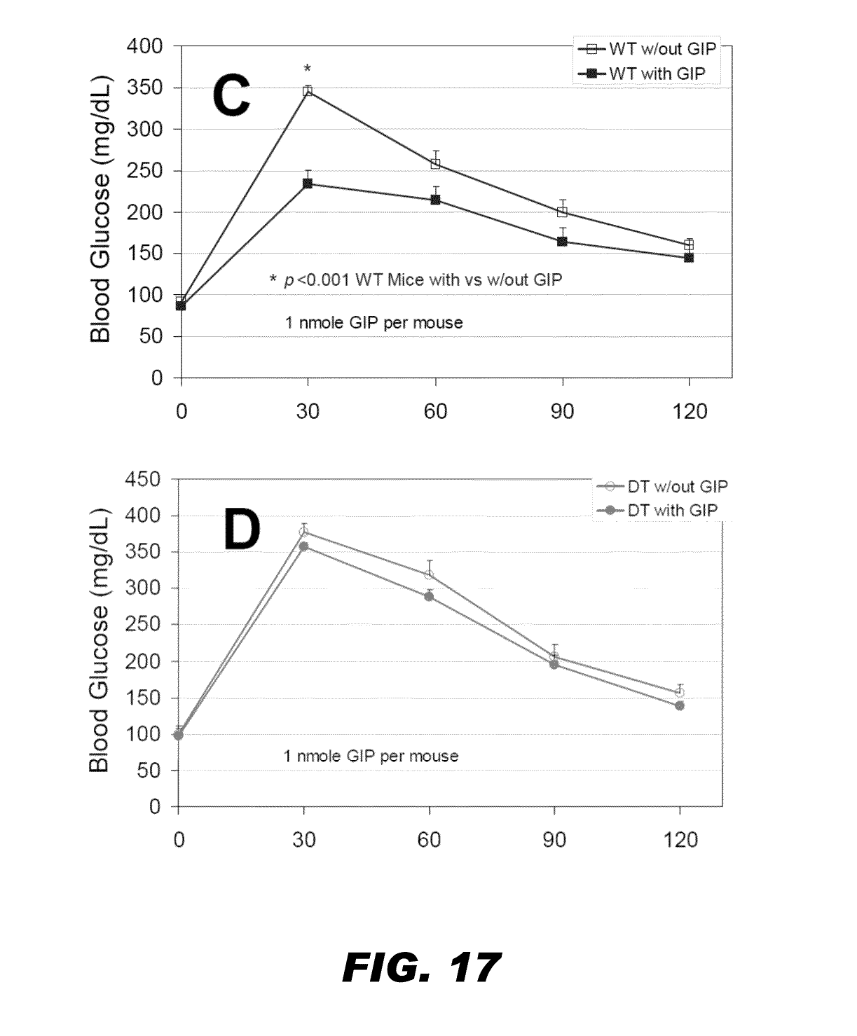

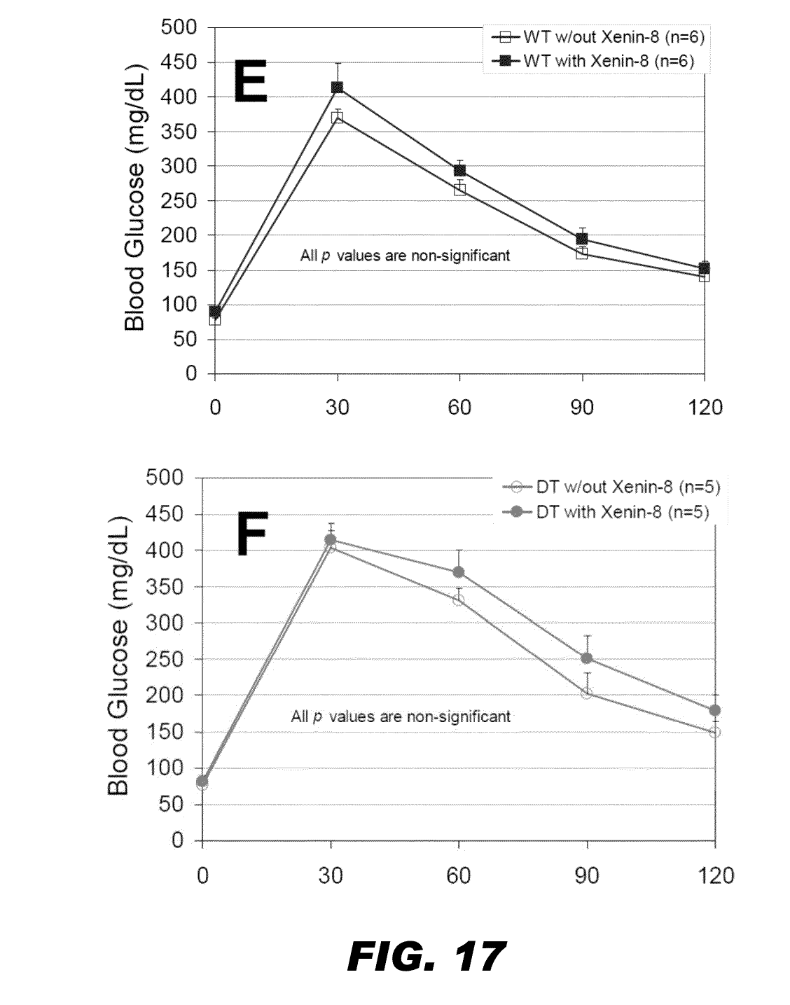

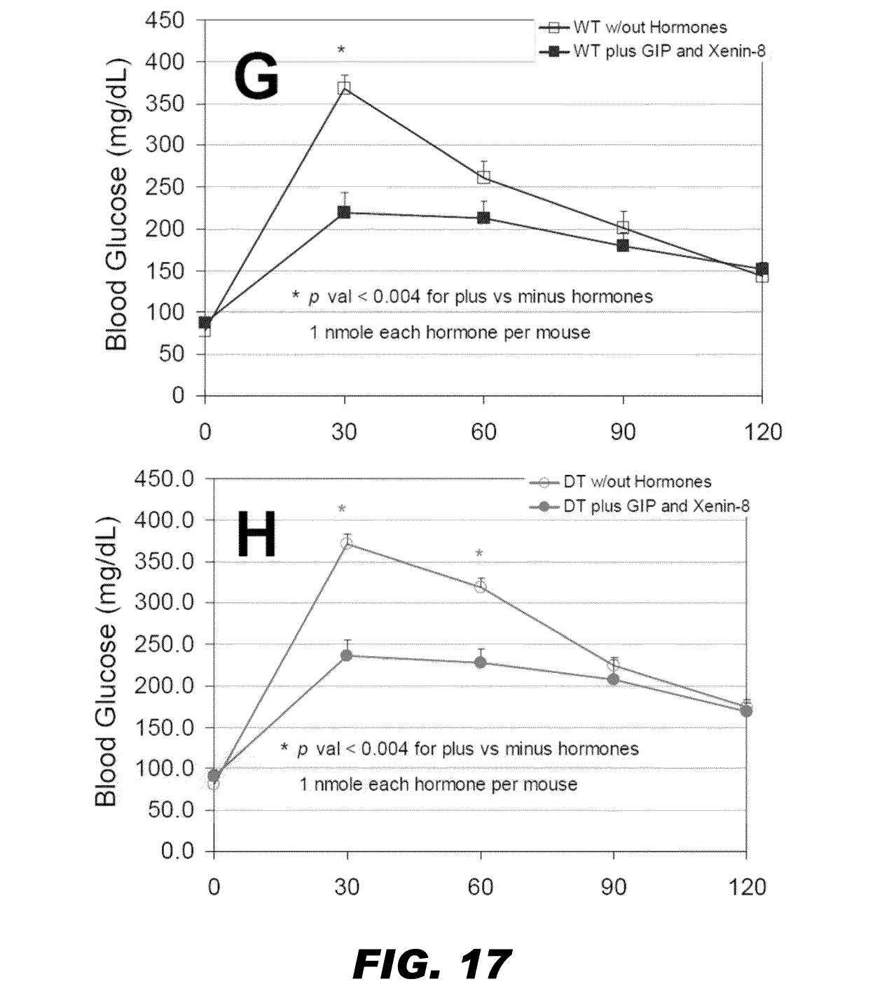

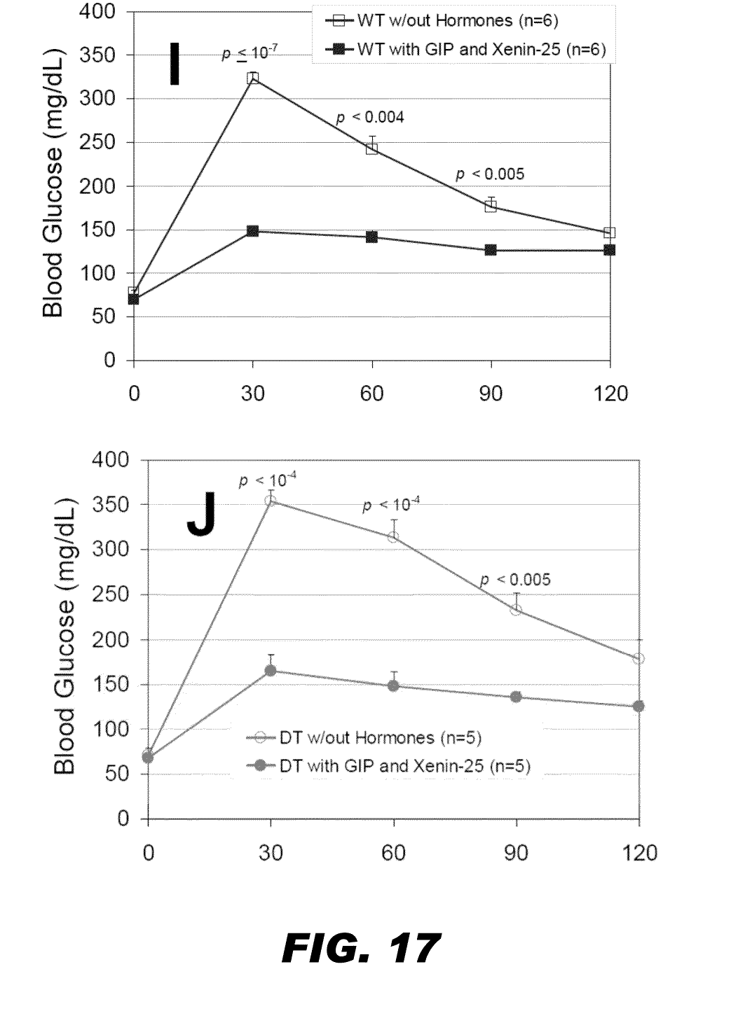

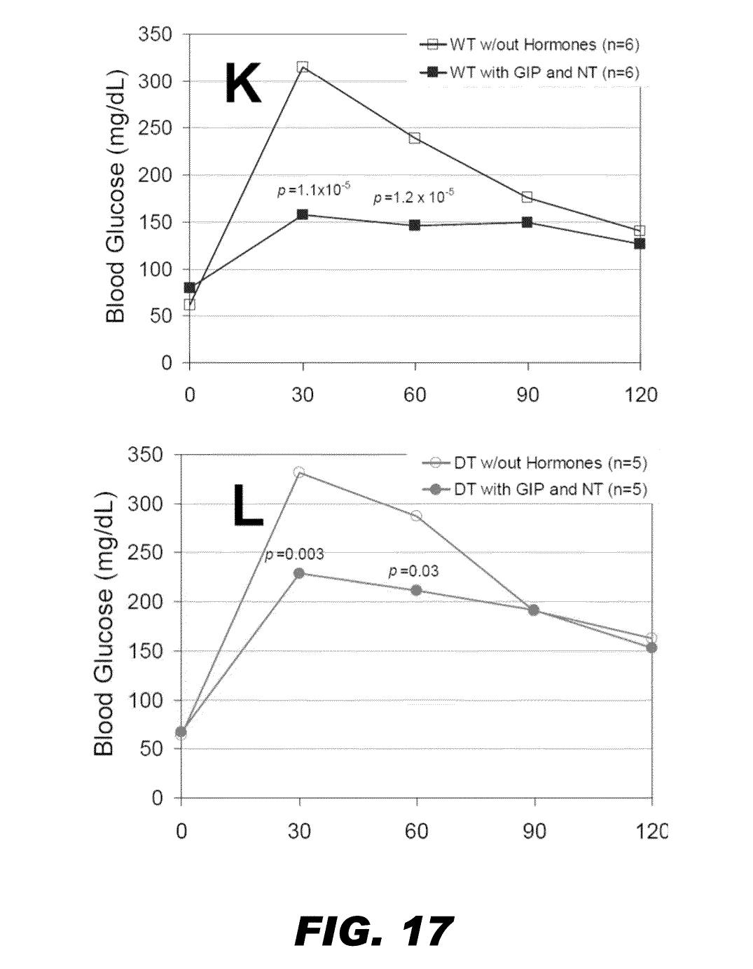

FIG. 17 depicts a series of graphs showing that GIP plus xenin, but not GIP or xenin alone, increases blood glucose clearance rates in mice lacking K cells. Wild type (WT) and GIP/DT (DT) mice were fasted overnight. Blood glucose levels were determined before (0 minutes) and at the indicated time after intraperitoneal administration of glucose (1 mg per g body weight) with or without 1 nmole of the indicated hormone(s). Note that: a) GLP-1 alone potentiates glucose clearance to similar extents in wild type and DT mice (panels A and B); b) GIP alone potentiates glucose clearance only in WT mice (panels C and D); c) Xenin-8 alone does not increase glucose clearance in WT or DT mice (panels E and F); d) A combination of GIP plus Xenin-8 or Xenin-25 potentiates glucose clearance in DT mice (panels G-J); e) Neurotensin (NT) only partially restores the GIP-mediated incretin effect in DT mice (panels K and L).

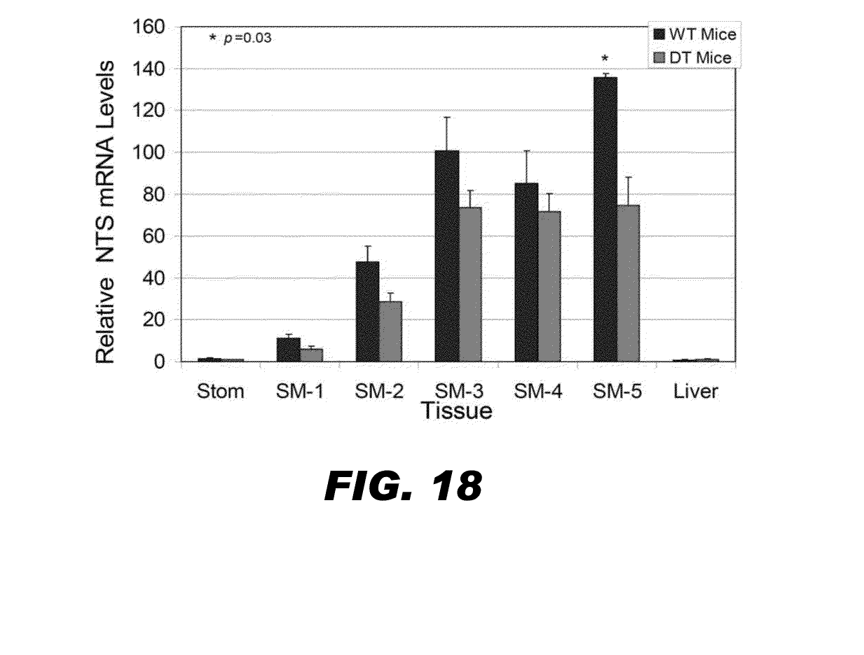

FIG. 18 depicts a graph showing that neurotensin (NT) mRNA levels are similar in the intestine of Wild type (WT) and GIP/DT (DT) mice. The mid-portion of sequential proximal to distal segments of mouse stomach (Stom) through small intestine (SM) were assayed for NT transcripts using real time PCR assays. SM-1 through SM-5 represent the most proximal (duodenum) through most distal (ileum), respectively, segments of small intestine. Note that NT mRNA levels are similar in both lines of mice. Liver served as a negative control.

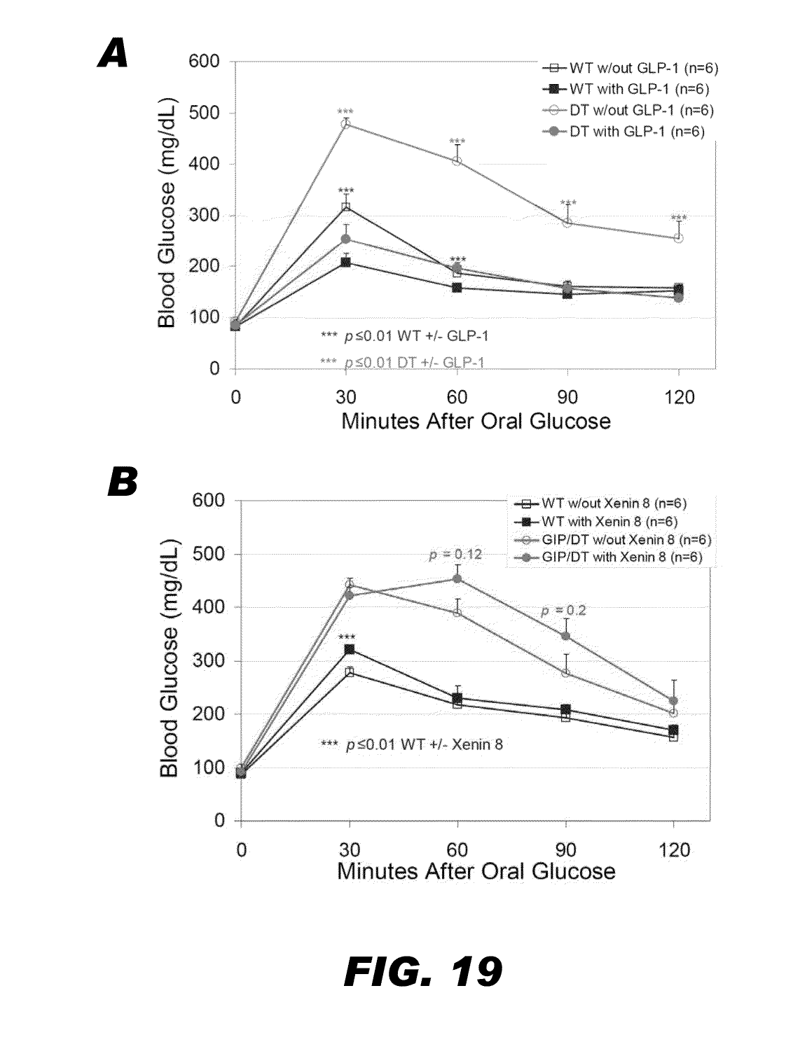

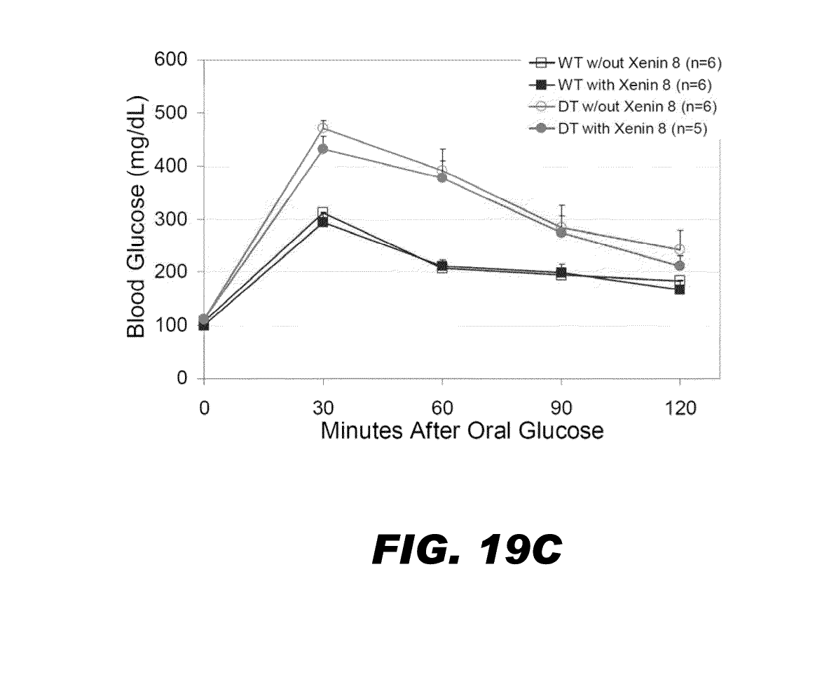

FIG. 19 depicts a graph showing that xenin does not increase the endogenous GLP-1-mediated incretin effect. Mice were fasted for 16-hours before they received glucose by intragastric gavage plus the indicated hormone via i.p. injection. Blood glucose levels were determined before and at the indicated time after administration of glucose and peptides. Note that: a) WT mice do not exhibit a robust response to GLP-1 alone because they already produce GLP-1, GIP, and Xenin; b) DT mice respond to GLP-1 because they do not produce GIP or Xenin and endogenous GLP-1 activity in WT and DT mice is low; c) Xenin alone has no effect on oral glucose clearance suggesting that it only potentiates the GIP-mediated incretin response.

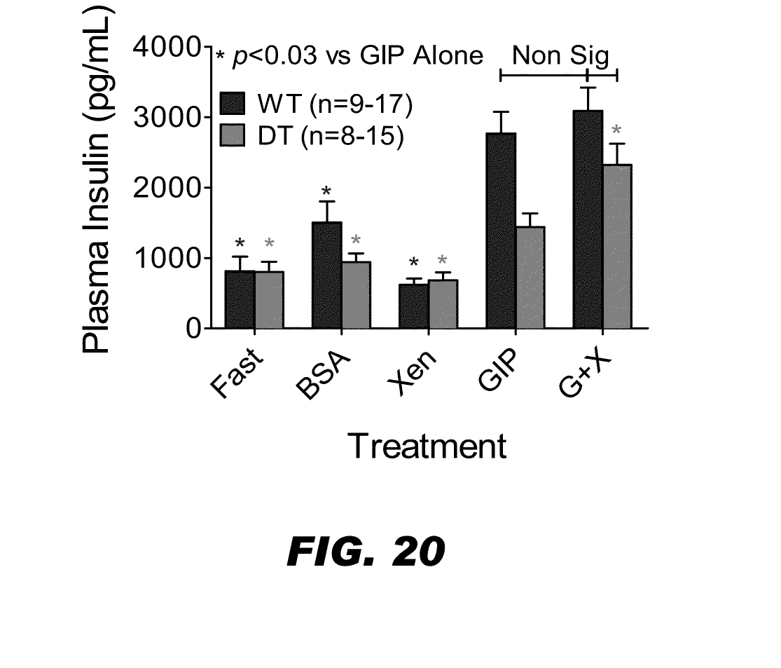

FIG. 20 depicts a graph showing that xenin-25 potentiates GIP-mediated insulin release in GIP/DT mice.

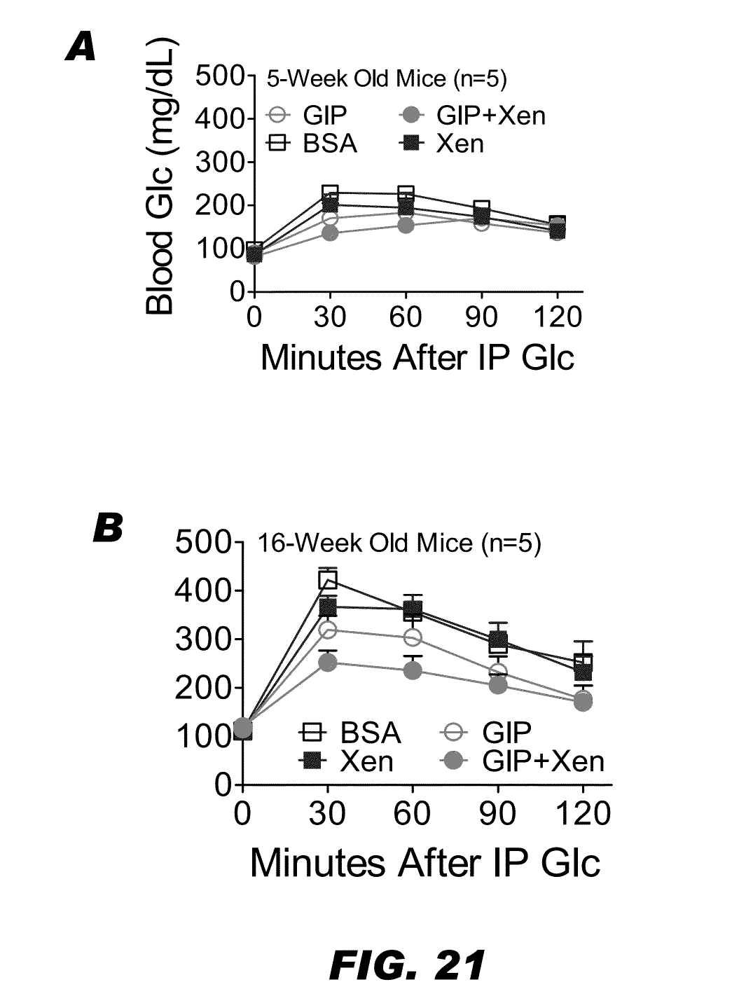

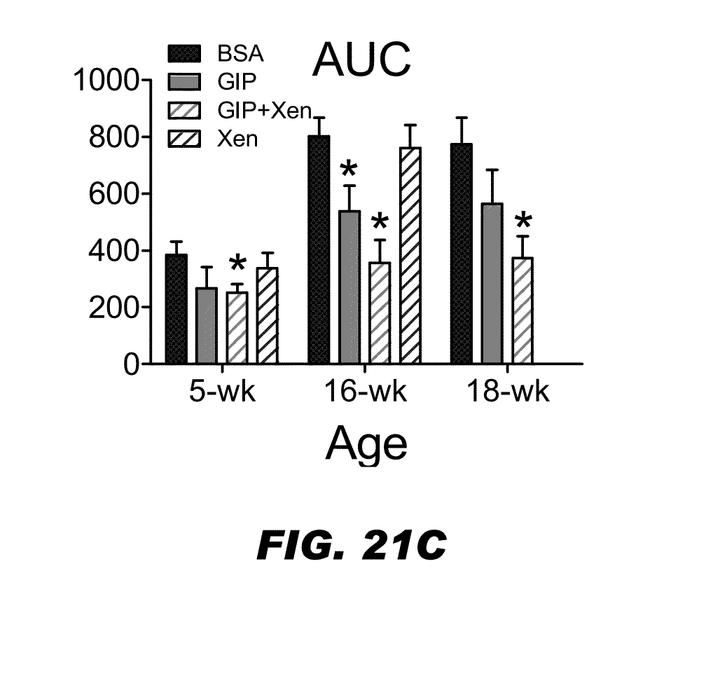

FIG. 21 depicts a series of graphs showing that Xenin-25 potentiates GIP action in a mouse model of human T2DM. Panel A depicts blood glucose levels in 5-week old pre-diabetic mice. Panel B depicts blood glucose levels in 16-week old diabetic mice. Panel C depicts the area under the curve for data in panels A and B as well as for a third experiment using 18-week old mice. * indicates P value<0.05 versus mice of the same age receiving BSA alone.

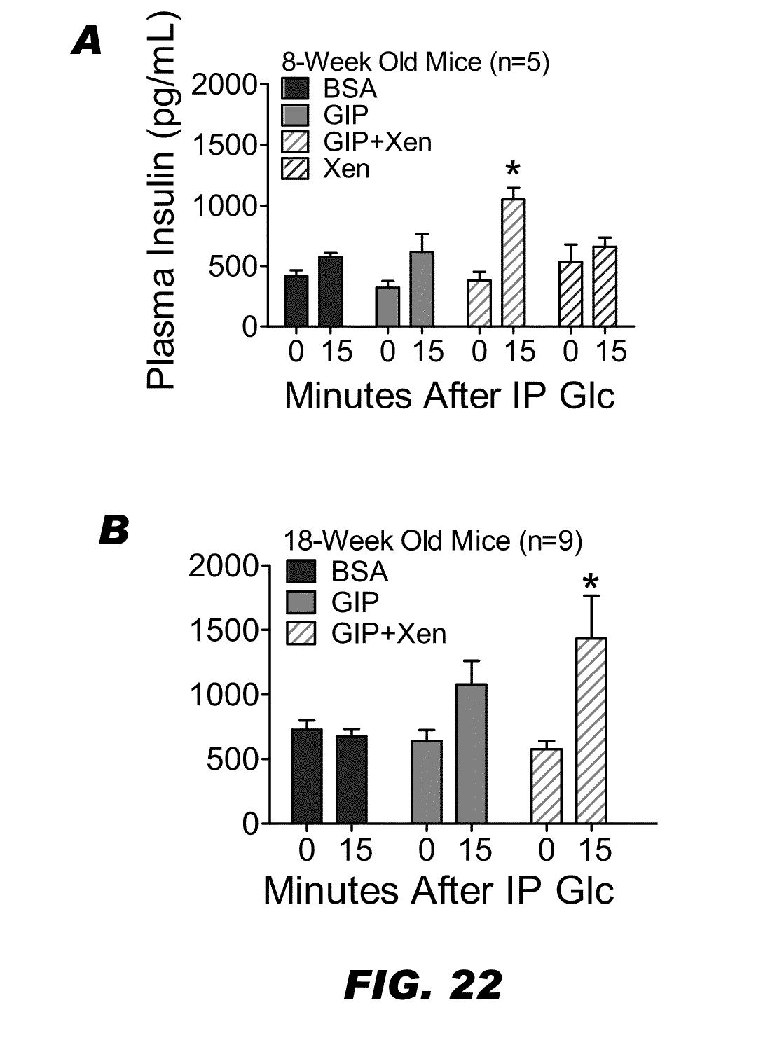

FIG. 22 depicts a series of graphs showing that xenin-25 potentiates GIP-mediated insulin release in a mouse model of human T2DM. Panel A depicts plasma insulin levels in 8-week old pre-diabetic mice. Panel B depicts plasma insulin levels in 18-week old mice. * indicates P value=0.032 and 0.05 versus fasting value and 15 minute value in animals receiving BSA, respectively.

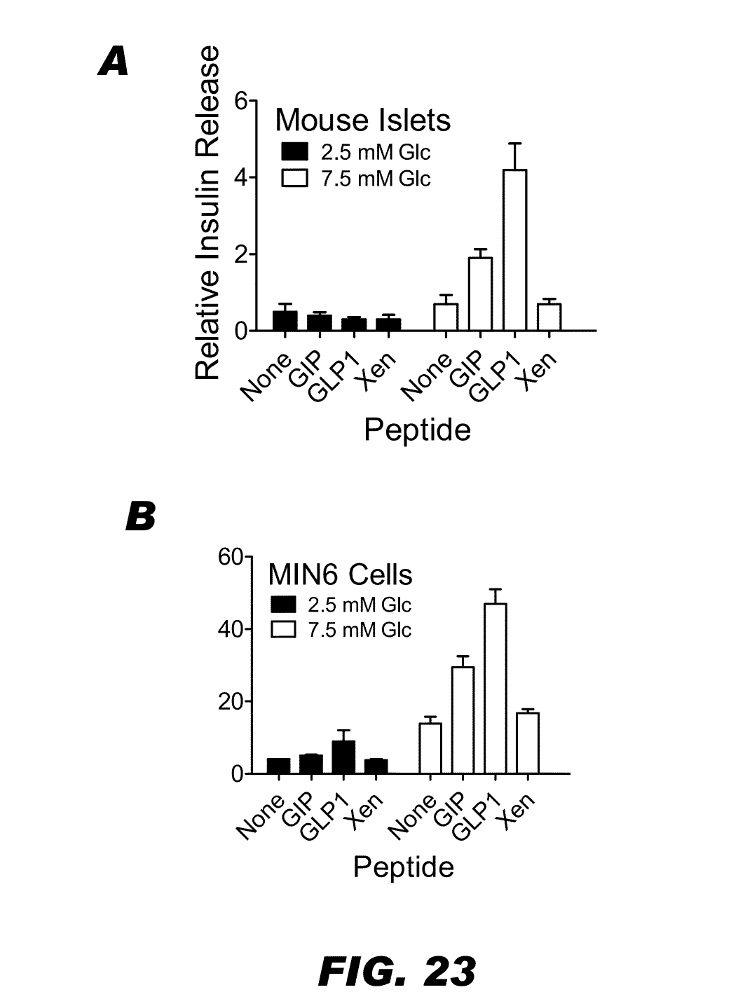

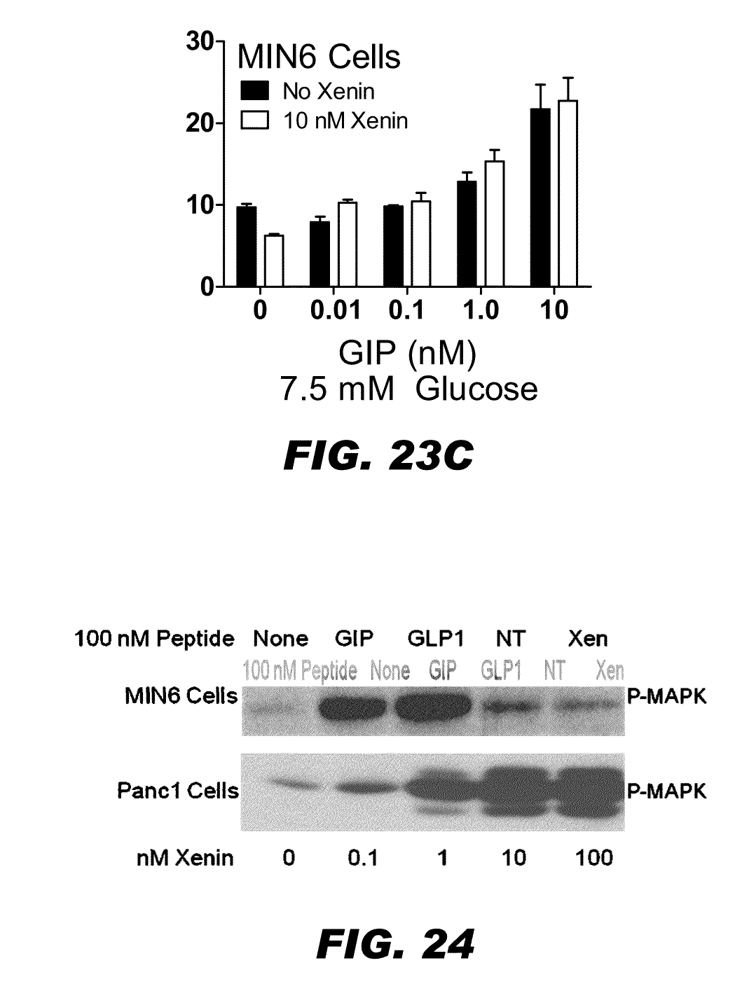

FIG. 23 shows that Xenin-25 does not directly stimulate insulin release. Insulin release assays (static incubations) were conducted at the indicated concentration of glucose with islets isolated from WT C57BL/6J mice (Panel A; 5 islets per sample; 6 samples per condition) or Min6 cells (Panels B, C; 4 wells per condition). 100 nM GIP, GLP-1, or Xenin-25 (Xen) was added to some samples in Panels A, B. Peptides were added at the indicated concentrations in Panels C. Note that Xenin-25 alone does not increase glucose-mediated insulin release and fails to potentiate GIP-mediated insulin release.

FIG. 24 presents Western blot analysis showing that incretins, but not Xenin-25, increase MAPK signaling in Min6 Cells. Top Panel presents a Western blot of extracts of MIN6 cells cultured overnight in 2.5 mM glucose without serum and then treated for 10 minutes with 7.5 mM glucose with/without 100 nM GIP, GLP-1, Neurotensin (NT) or Xenin-25 (Xen). Cells were harvested and subjected to Westerns blot analysis using an antibody specific for phosphorylated (active) MAPK. Note that GIP and GLP-1, but not NT or Xenin-25, activate MAPK. Bottom Panel presents a Western blot of extracts of Panc-1 cells incubated overnight in the absence of serum and then treated for 10 minutes with the indicated concentration of Xenin-25. Cells were analyzed by Western blots as described in the top panel. Note that Xenin-25 profoundly increased phosphorylation of MAPK even at 1 nM.

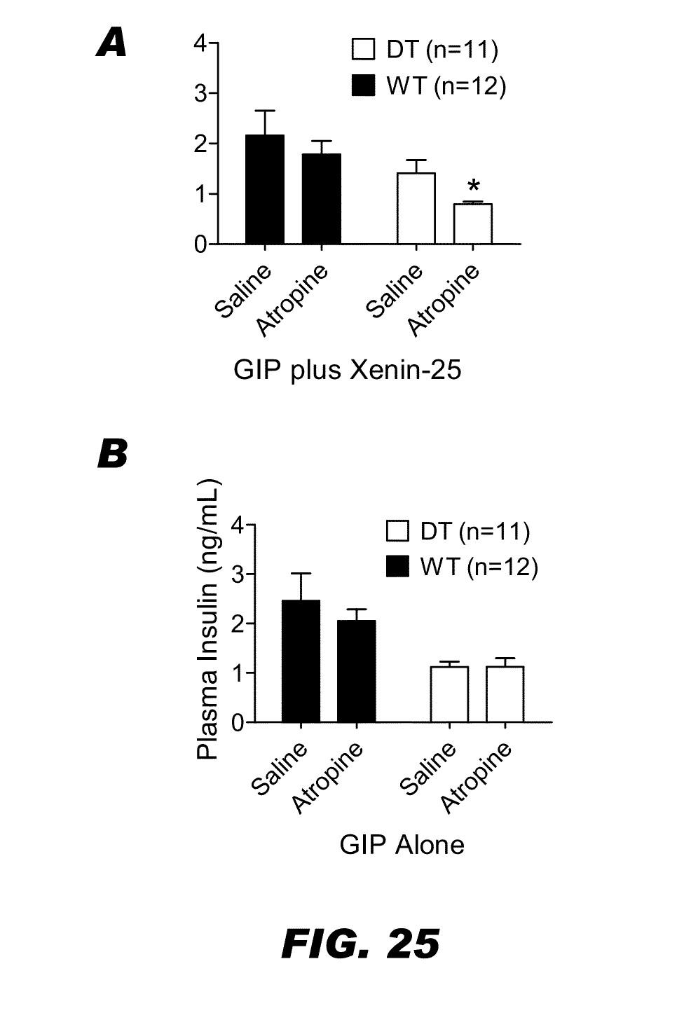

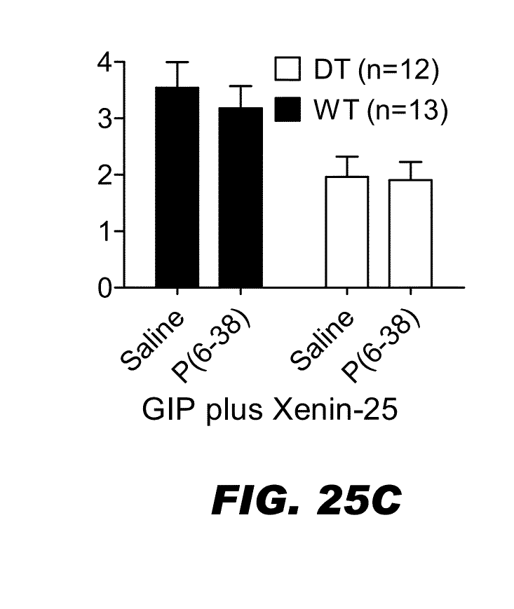

FIG. 25 depicts a graph showing that Xenin-25 potentiates GIP-mediated insulin release in vivo via an atropine sensitive pathway. WT and DT mice were fasted for 16-h (Fast). As indicated, some mice were then administered an intraperitoneal injection of glucose (1 g/kg) with vehicle alone (BSA), GIP alone, Xenin-25 alone (Xen) or GIP plus Xenin-25 (G+X). As indicated, some animals also received an intraperitoneal injection of atropine (500 micrograms/kg) or saline 15 minutes before administration of peptides (Panels A and B) or PACAP PACAP(6-38) [P6-38)] or saline along with peptides (Panel C). Five minutes after injection of glucose, blood was collected and plasma prepared for insulin assays. Note that the insulin response to GIP is blunted in the DT mice and Xenin-25 potentiates this response in DT, but not WT, animals (FIG. 20). Furthermore, atropine reduces the insulin response to GIP plus Xenin-25 but not GIP alone and P(6-38) does not inhibit the insulin response to GIP plus Xenin-25.

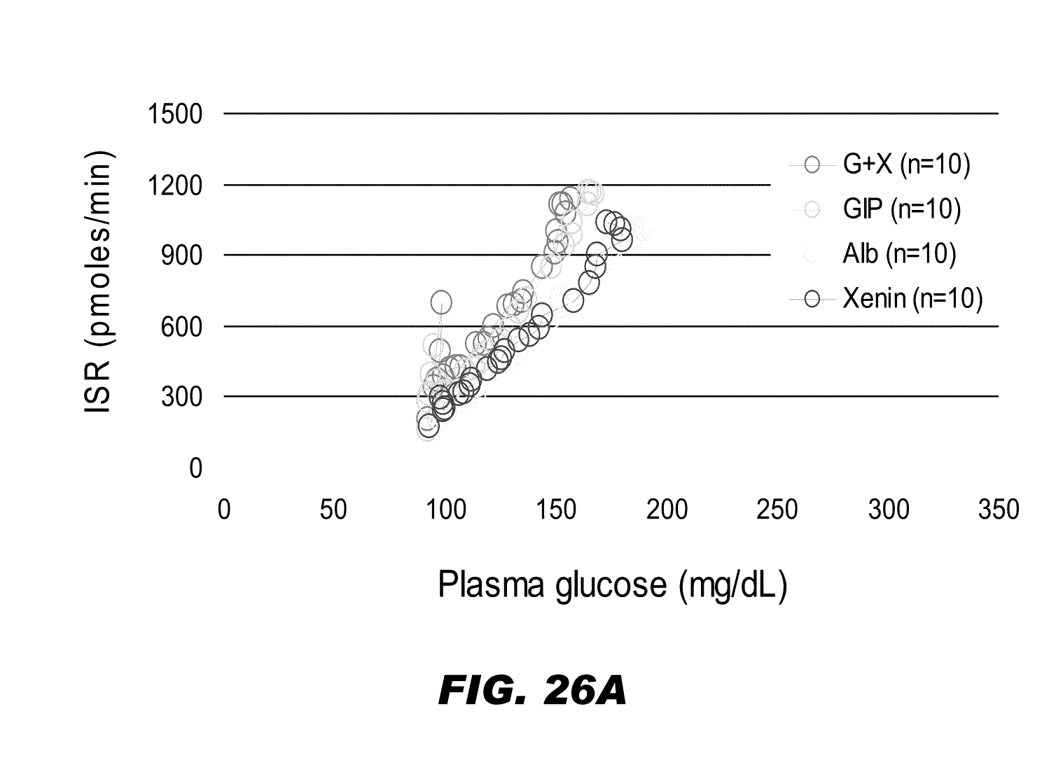

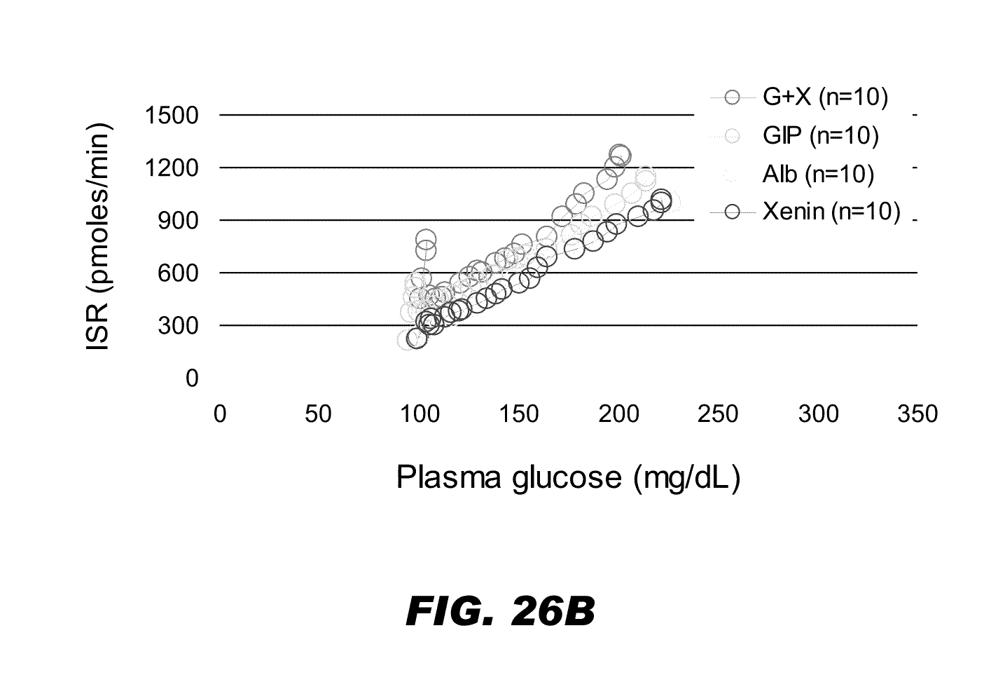

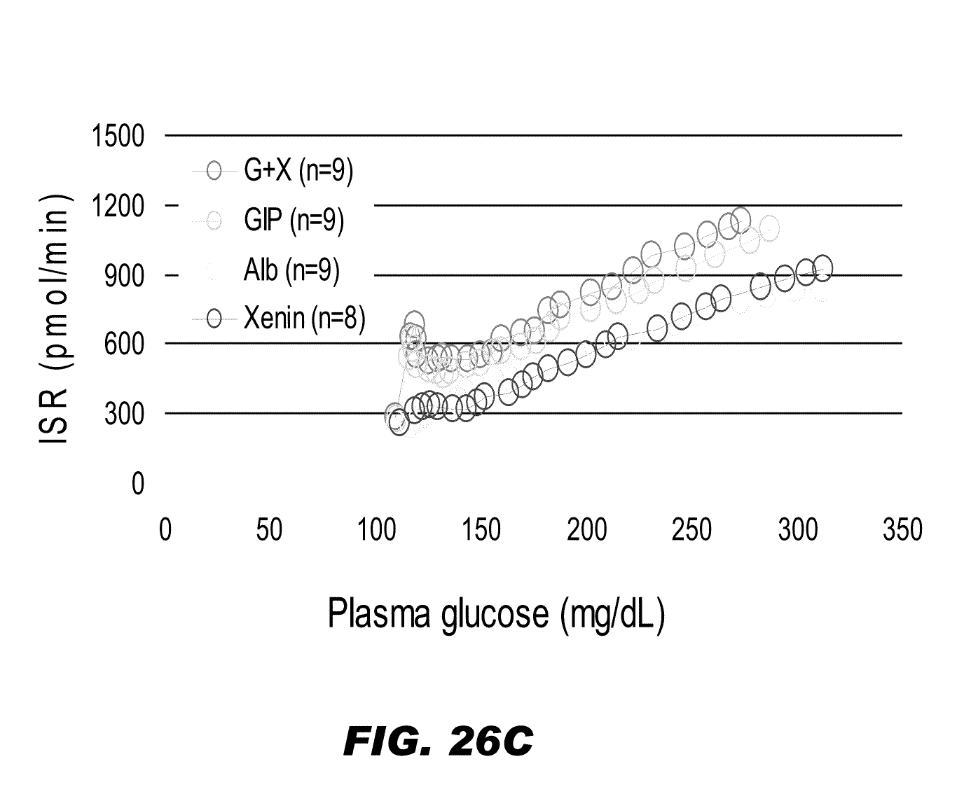

FIG. 26 depicts three graphs showing the insulin secretion rate (ISR) vs. plasma glucose during a graded glucose infusion (GGI) in subjects with (A) normal glucose tolerance, (B) impaired glucose tolerance, and (C) type II diabetes.

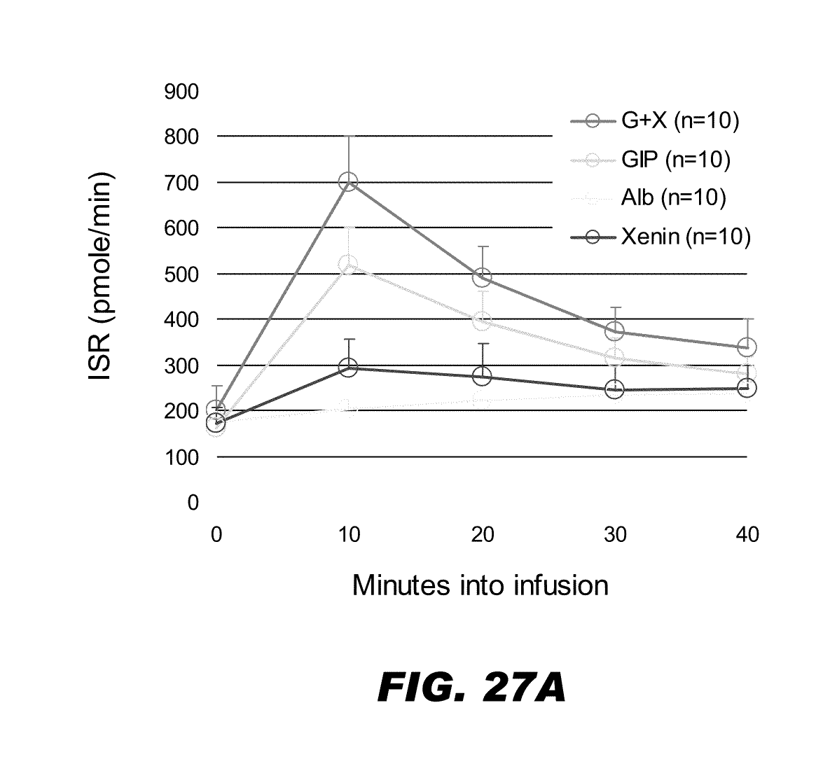

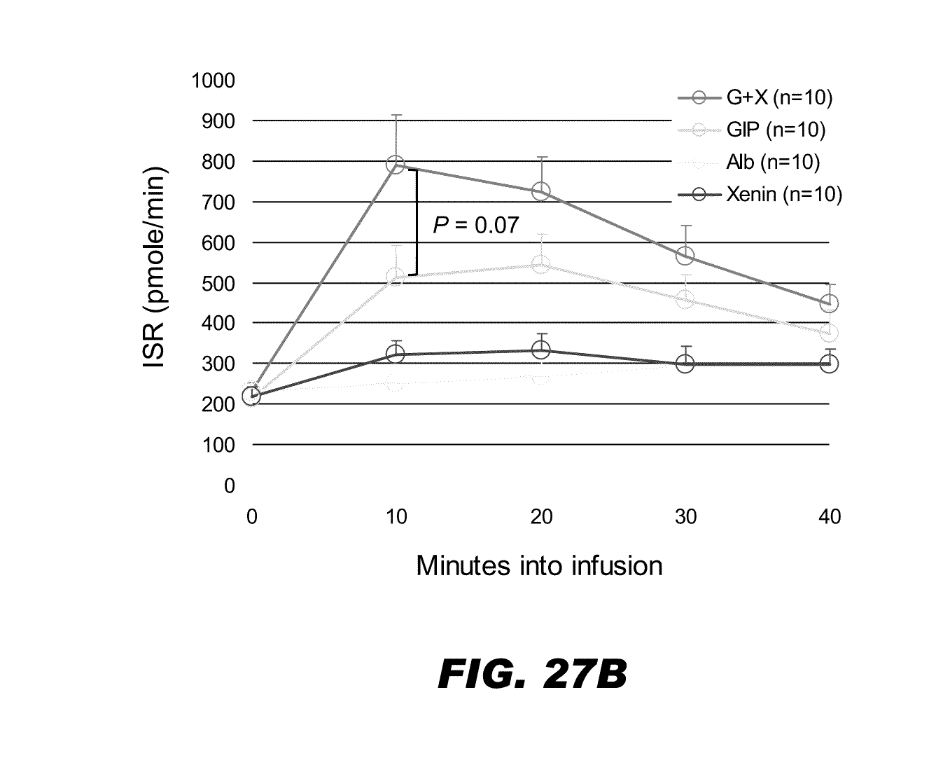

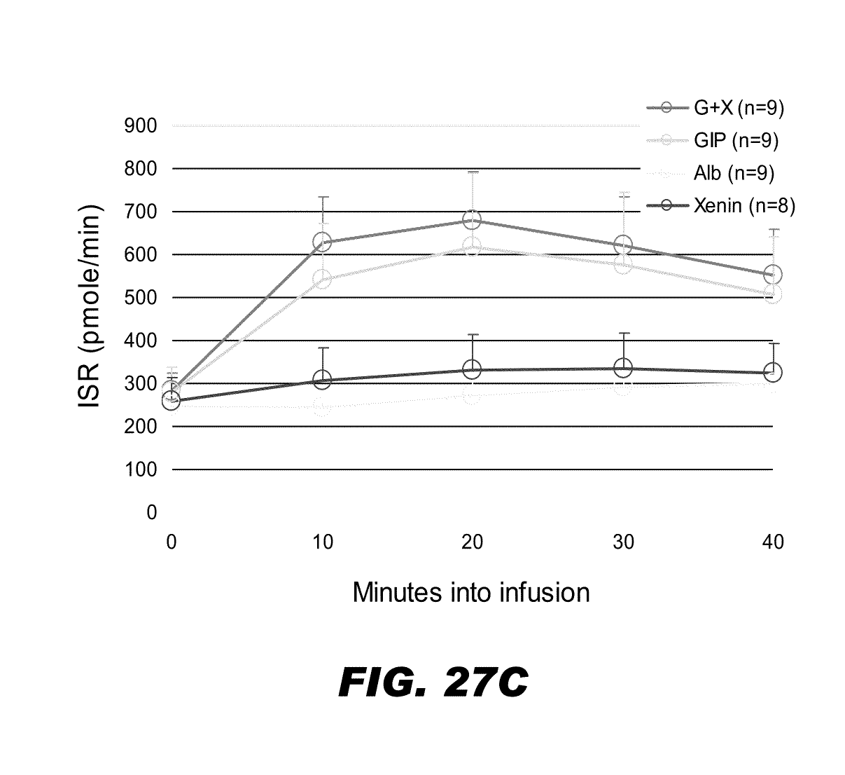

FIG. 27 depicts three graphs showing the ISR during the first 40 min of a GGI in subjects with (A) normal glucose tolerance, (B) impaired glucose tolerance, and (C) type II diabetes.

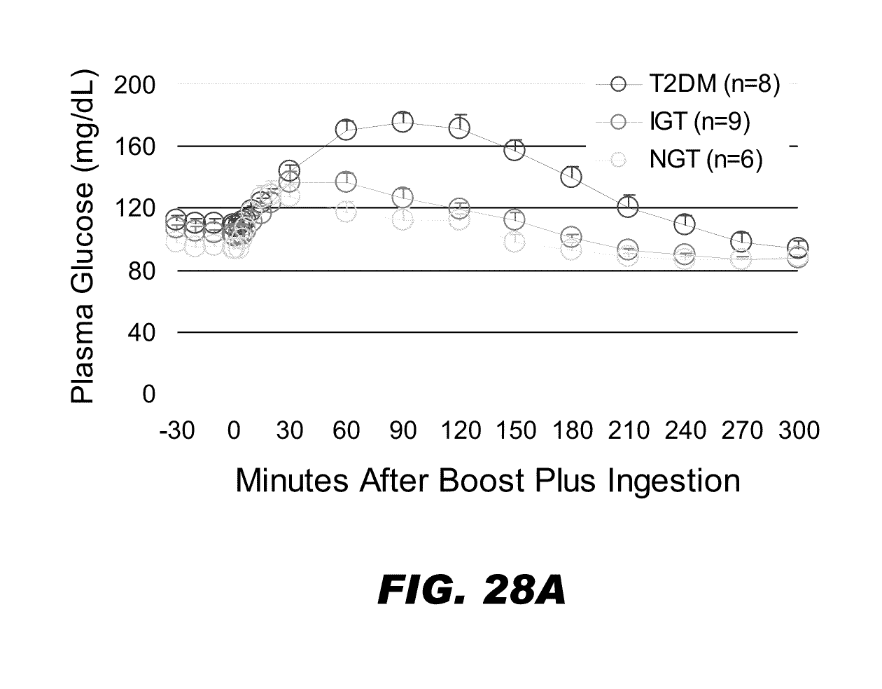

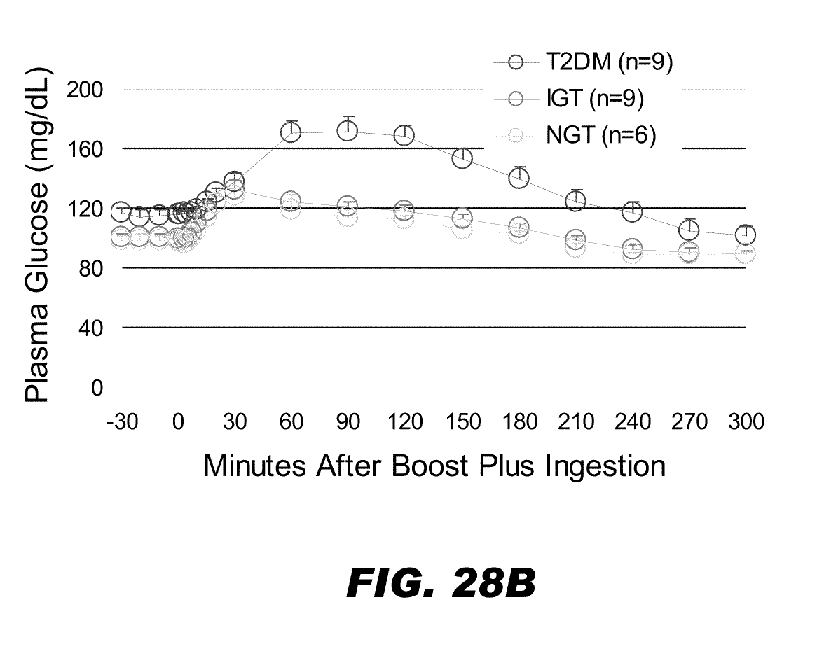

FIG. 28 depicts two graphs showing plasma glucose levels during a meal tolerance test (A) without peptides and (B) with a xenin-25 infusion.

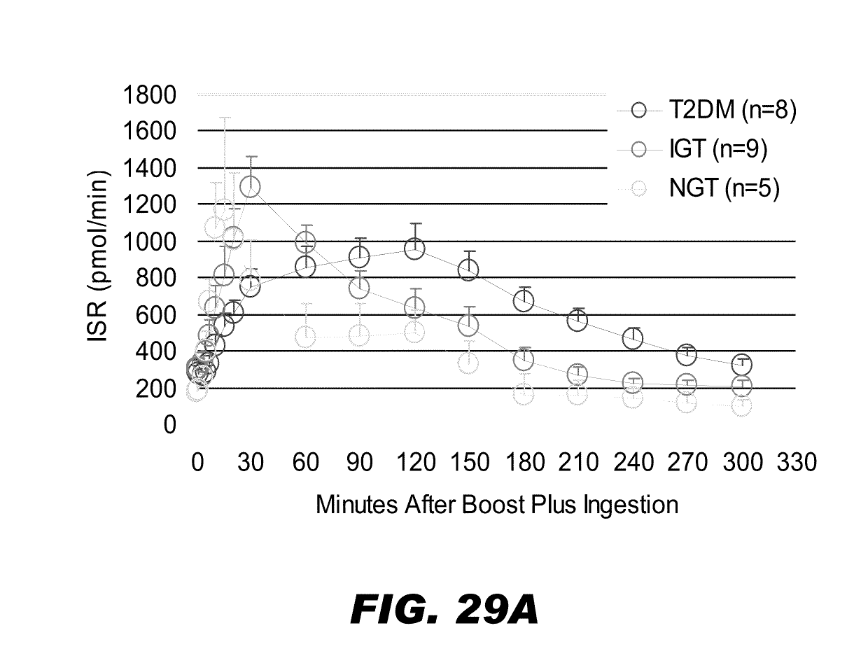

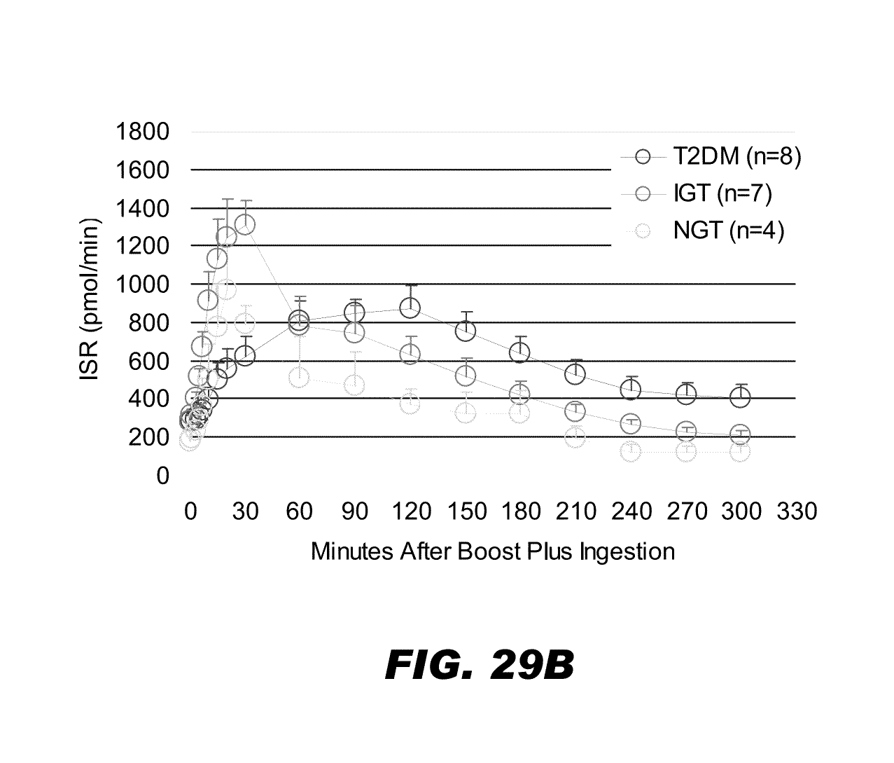

FIG. 29 depicts two graphs showing insulin secretion rates during a meal tolerance test (A) without peptides and (B) with a xenin-25 infusion.

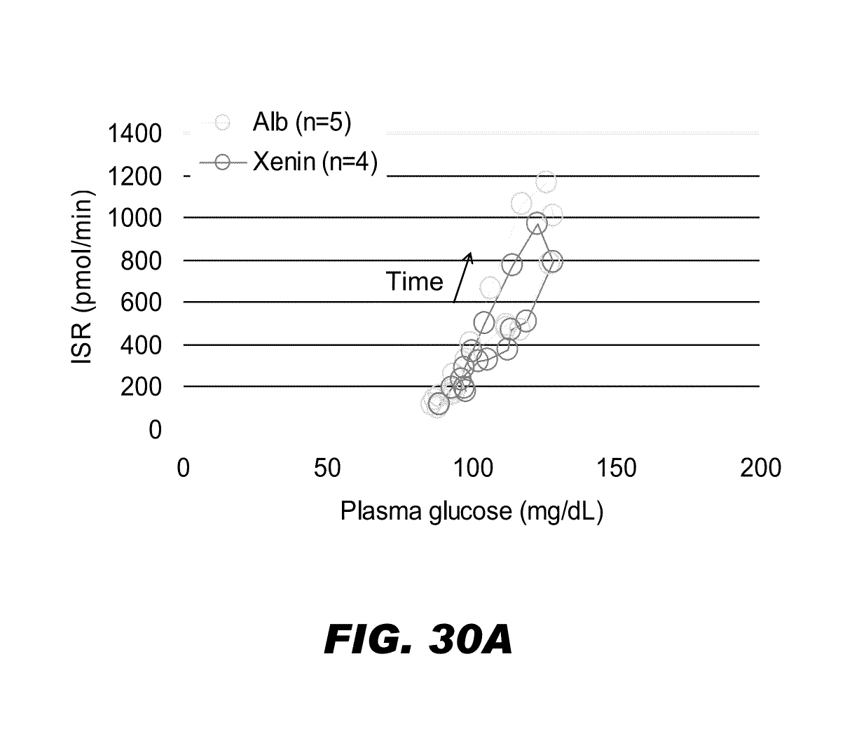

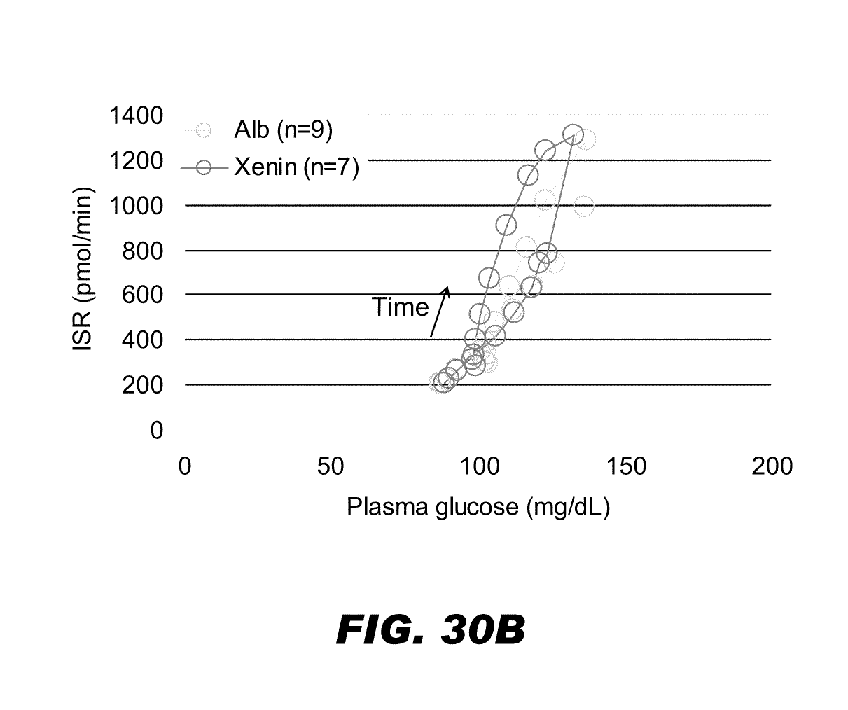

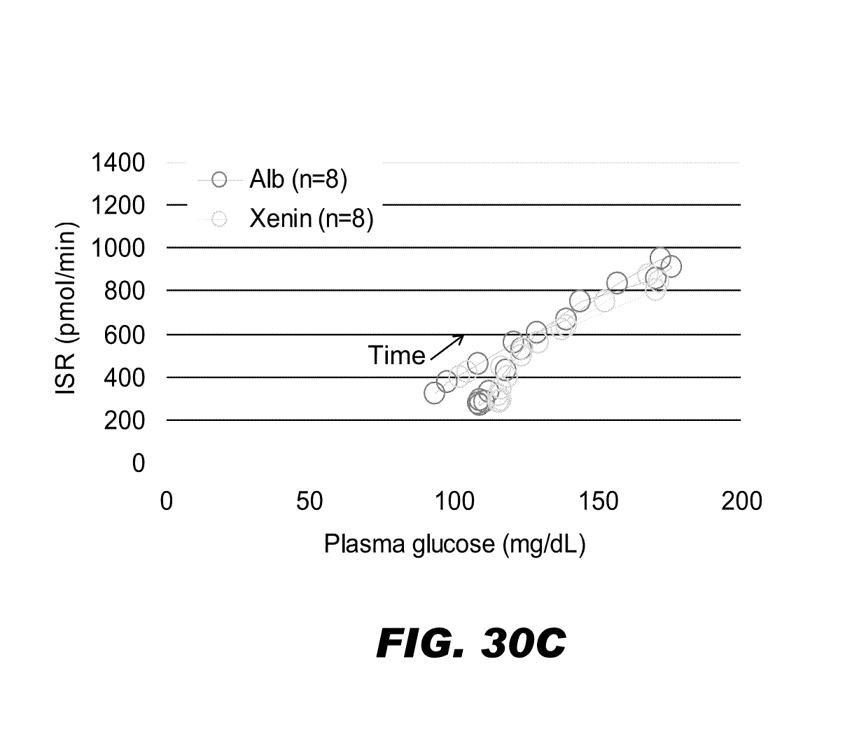

FIG. 30 depicts three graphs showing ISR v. plasma glucose levels during a meal tolerance test with and without a xenin-25 infusion in subjects with (A) normal glucose tolerance, (B) impaired glucose tolerance, and (C) type II diabetes.

### DETAILED DESCRIPTION OF THE INVENTION

The present invention provides a combination and a method that may be used to modulate GIP activity in a subject. The invention is based on the discovery that the protein xenin potentiates GIP activity. Consequently modulating xenin activity in a subject may in turn modulate GIP activity.

### I. Combinations for Insulin Modulation

One aspect of the present invention encompasses a combination that may be beneficially used to modulate insulin. In one embodiment, the combination comprises a compound capable of modulating xenin activity and at least one compound capable of modulating insulin secretion. In another embodiment, the combination comprises a compound capable of modulating xenin activity and at least one compound capable of modulating weight gain. As used herein, compound may refer to a biomolecule such as a protein, lipid, carbohydrate, nucleic acid or combination thereof such as a lipoprotein or a glycoprotein, a small molecule, or an antibody or fragment thereof. In each of the above embodiments, the molar ratio between the compound capable of modulating xenin activity and at least one compound capable of modulating insulin secretion or weight gain can and will vary depending on the selection of components comprising the combination. In an exemplary embodiment, the ratio that provides the greatest therapeutic benefit to the subject is generally used.

### (a) Compounds Capable of Modulating Xenin Activity.

In one embodiment, the invention encompasses a combination comprising a compound capable of modulating xenin activity. As used herein, “modulating” may refer to increasing or decreasing xenin activity in a subject. The phrase “xenin activity,” as used herein, may refer to the concentration of xenin mRNA, the concentration of xenin protein, and/or xenin's ability to potentiate the incretin activity of GIP or GIP's activity in adipocytes.

Methods of detecting and quantifying the concentration of xenin mRNA is known in the art. Methods of detecting and quantifying the concentration of xenin protein are known in the art. For instance, see Feurle et al., (1992) 267 (31):22305-09, hereby incorporated by reference in its entirety. As used herein, “xenin protein” refers to xenin-25, and active fragments thereof, such as xenin-8. Methods of detecting and quantifying xenin's ability to facilitate the incretin activity of GIP are detailed in the Examples below. Methods that may be used to detect and quantify xenin's ability to facilitate GIP's activity in adipocytes are known in the art. For instance, see Song et al., (2007) Gastroenterology, 133 (6):1796-1805.

In one embodiment, xenin activity is increased. Xenin activity may be increased by increasing the concentration of xenin mRNA. This may be through increasing the copy number of xenin mRNA, increasing the stability of xenin mRNA, or decreasing the degradation of xenin mRNA using techniques commonly known in the art.

Xenin activity may also be increased by increasing the concentration of xenin protein. This may be through increasing the amount of xenin protein such as xenin-25 or xenin-8, increasing the amount of proxenin, increasing the stability of xenin protein, or decreasing the degradation of xenin protein. For instance, the amount of xenin protein may be increased by administering xenin protein to a subject. A number of xenin proteins known in the art are suitable for use in the present invention. Generally speaking, the xenin protein is from a mammal. In certain aspects, a protein that is a homolog, ortholog, mimic or degenerative variant of a xenin protein is also suitable for use in the present invention. In an exemplary embodiment, the xenin protein administered to the subject is modified to increase the half-life of the protein by increasing the stability of the protein or decreasing the degradation of the protein. A number of methods may be employed to determine whether a particular homolog, mimic or degenerative variant possesses substantially similar biological activity relative to a xenin protein. For instance, activity may be determined by detecting and/or quantifying the effect of xenin on GIP activity.

In addition to having a substantially similar biological function, a homolog, ortholog, mimic or degenerative variant suitable for use in the invention will also typically share substantial sequence similarity to a xenin protein. In addition, suitable homologs, ortholog, mimic or degenerative variants preferably share at least 30% sequence homology with a xenin protein, more preferably, 50%, and even more preferably, are greater than about 75% homologous in sequence to a xenin protein. Alternatively, peptide mimics of xenin could be used that retain critical molecular recognition elements, although peptide bonds, side chain structures, chiral centers and other features of the parental active protein sequence may be replaced by chemical entities that are not native to xenin protein yet, nevertheless, confer activity.

In determining whether a polypeptide is substantially homologous to a xenin polypeptide, sequence similarity may be determined by conventional algorithms, which typically allow introduction of a small number of gaps in order to achieve the best fit. In particular, “percent homology” of two polypeptides or two nucleic acid sequences is determined using the algorithm of Karlin and Altschul [(Proc. Natl. Acad. Sci. USA 87, 2264 (1993)]. Such an algorithm is incorporated into the NBLAST and XBLAST programs of Altschul, et al. (J. Mol. Biol. 215, 403 (1990)). BLAST nucleotide searches may be performed with the NBLAST program to obtain nucleotide sequences homologous to a nucleic acid molecule of the invention. Equally, BLAST protein searches may be performed with the XBLAST program to obtain amino acid sequences that are homologous to a polypeptide of the invention. To obtain gapped alignments for comparison purposes, Gapped BLAST is utilized as described in Altschul, et al. (Nucleic Acids Res. 25, 3389 (1997)). When utilizing BLAST and Gapped BLAST programs, the default parameters of the respective programs (e.g., XBLAST and NBLAST) are employed. See www.ncbi.nlm.nih.gov for more details.

Xenin proteins suitable for use in the invention are typically isolated or pure and are generally administered as a composition in conjunction with a suitable pharmaceutical carrier, as detailed below. A pure polypeptide constitutes at least about 90%, preferably, 95% and even more preferably, at least about 99% by weight of the total polypeptide in a given sample.

The xenin protein may be synthesized, produced by recombinant technology, or purified from cells using any of the molecular and biochemical methods known in the art that are available for biochemical synthesis, molecular expression and purification of the xenin proteins [see e.g., Molecular Cloning, A Laboratory Manual (Sambrook, et al. Cold Spring Harbor Laboratory), Current Protocols in Molecular Biology (Eds. Ausubel, et al., Greene Publ. Assoc., Wiley-Interscience, New York)].

Alternatively, the amount of xenin protein may be increased by administering a compound that inhibits the degradation of xenin protein.

Additionally, xenin activity may be increased by increasing xenin's ability to potentiate the incretin activity of GIP. For instance, a xenin homologue that binds to a receptor with greater affinity than wild-type xenin may increase xenin's ability to potentiate the incretin activity of GIP. By way of non-limiting example, a pseudopeptide analog Y (CH₂NH) hexapeptide in which a Y reduced bond was introduced into the biologically important dibasic motif of the C-terminus of xenin exhibited increased biological activity with respect to secretion from the exocrine pancreas when compared to xenin-6 (Feurle et al., 2003, Life Sciences 74:697)]. Or alternatively, an antibody agonist may increase xenin's ability to potentiate the incretin activity of GIP.

Similarly, xenin activity may be increased by increasing xenin's ability to modulate GIP's activity in adipocytes. For instance, a xenin homologue that binds to a receptor with greater affinity than wild-type xenin may increase xenin's ability to modulate GIP's activity in adipocytes. Or alternatively, an antibody agonist may increase xenin's ability to modulate GIP's activity in adipocytes.

In another embodiment, xenin activity is decreased. Xenin activity may be decreased by decreasing the concentration of xenin mRNA. This may be through decreasing the copy number of xenin mRNA, decreasing the stability of xenin mRNA, or increasing the degradation of xenin mRNA using techniques commonly known in the art.

Xenin activity may also be decreased by decreasing the concentration of xenin protein. This may be through decreasing the amount of xenin protein, decreasing the stability of xenin protein, or increasing the degradation of xenin protein. For instance, the amount of xenin protein may be decreased by administering a compound that increases the degradation of xenin protein. In a further embodiment, the amount of proxenin may be decreased.

Additionally, xenin activity may be decreased by decreasing xenin's ability to potentiate the incretin activity of GIP. For instance, a molecule that blocks the binding of xenin to a receptor may decrease xenin's ability to potentiate the incretin activity of GIP. Or alternatively, an antibody antagonist may decrease xenin's ability to potentiate the incretin activity of GIP.

Similarly, xenin activity may be decreased by decreasing xenin's ability to facilitate the incretin activity of GIP. For instance, a molecule that blocks the binding of xenin to a receptor may decrease xenin's ability modulate GIP's activity in adipocytes. Or alternatively, an antibody antagonist may decrease xenin's ability modulate GIP's activity in adipocytes.

The amount of a compound capable of modulating xenin activity comprising a single dosage of the combination will vary depending upon the subject, the compound, and the particular mode of administration. In an illustrative example, xenin-25 may be administered intravenously at doses of 0.5 to 5.0 pmoles×kg⁻¹×min⁻¹ or up to 260 pmoles×kg⁻¹×min⁻¹ for a duration of up to 5 hours. Alternatively, a xenin-25 may be administered in an oral or IV bolus. Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

### (b) Compounds Capable of Modulating Insulin Secretion

In one embodiment, the invention encompasses a combination comprising at least one compound capable of modulating insulin secretion. As used herein, “modulating” may refer to increasing or decreasing insulin secretion in a subject. In some embodiments, the combination comprises at least two, at least three, or at least four compounds capable of modulating insulin secretion. Methods of detecting and quantifying insulin secretion are known in the art. For instance, see the Examples.

In some embodiments, the compound capable of modulating insulin secretion may be a GLP protein or a GLP protein homologue. For instance, the compound may be GLP-1(7-37) or GLP-1(7-36) amide. Alternatively, the compound may be a GLP homologue that possesses a longer pharmacological half-life. For instance, the GLP homologue may be resistant to cleavage by dipeptidyl peptidase IV (DPP-IV). Such homologues are known in the art. Methods of determining whether a protein is a homologue or analogue to GLP are known in the art, and detailed above with respect to xenin. Non-limiting examples include exendin-4 (also known as exenatide), and NN2211 (also known as liraglutide). Additional examples may be found in US Patent application no. 2004/0127414, 2005/0059605, and 2006/0234933, each of which are hereby incorporated by reference in their entirety. The compound may also be a GLP-1 receptor agonist, such as an antibody agonist or a small molecule agonist. Additionally, the compound may also be a GLP-1 receptor antagonist, such as an antibody antagonist or a small molecule antagonist.

The amount of GLP-1 capable of modulating insulin secretion comprising a single dosage of the combination will vary depending upon the subject, the compound, and the particular mode of administration. In an illustrative example, GLP-1 may be administered intravenously at doses of 0.1 to 5.0 pmoles×kg⁻¹×min⁻¹ for a duration of 0.5 to 55 hours. Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

In other embodiments, the compound capable of modulating insulin secretion may be a GIP protein or a GIP protein homologue. Such homologues are known in the art. Methods of determining whether a protein is a homologue or analogue to GIP are known in the art, and detailed above with respect to xenin. For instance, the compound may be a truncated or modified GIP protein, such as GIP(6-30)amide, GIP(7-30)amide, or (Pro³)GIP. Additionally, the homologue may be resistant to cleavage by DPP-IV. Alternatively, the compound may also be a GIP agonist, such as an antibody agonist or a small molecule agonist. Similarly, the compound may also be a GIP antagonist, such as an antibody antagonist or a small molecule antagonist. In some embodiments, the GIP may be endogenous GIP, for instance, GIP secreted after food consumption by the subject. In other embodiment, the GIP may be exogenously administered GIP.

The amount of GIP capable of modulating insulin secretion comprising a single dosage of the combination will vary depending upon the subject, the compound, and the particular mode of administration. In an illustrative example, GIP may be administered intravenously at doses of 0.5 to 20 pmoles×kg⁻¹×min⁻¹ for a duration of 0.5 to 5 hours. Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

In certain embodiments, the compound capable of modulating insulin secretion may be a DPP-IV inhibitor. Such compounds may increase the pharmacological half-life of an incretin protein, homologue, or analogue. DPP-IV inhibitors are known in the art, and non-limiting examples may include P32/98, NVP DPP728, sitagliptin phosphate, vildagliptin, and LAF237. In addition, DPP-IV inhibitors may be found in US Patent application no. 2002/0110560, 2005/0107309, and 2005/0203030, each of which is hereby incorporated by reference in their entirety.

In other embodiments, the compound capable of modulating insulin secretion may be a parasympathomimetic drug. Products released from parasympathetic neurons are known to increase insulin release from pancreatic islet beta cells. And, the results described in Example 14 suggest that the effects of xenin-25 on GIP-mediated insulin release in vivo are mediated by products released from parasympathetic neurons. Parasympathomimetic drugs, also known as cholinergic drugs or agents or agonists, are known in the art, and may include acetylcholine precursors and cofactors, acetylcholine receptor agonists and cholinergic enzymes. Non-limiting examples of parasympathomimetic drugs may include muscarine, pilocarpine, nicotine, suxamethonium, Dyflos, ecothiopate, physostigmine and neostigmine.

In further embodiments, the compound capable of modulating insulin secretion may be a compound used to treat diabetes. For instance, the compound may be used to treat type II diabetes. Non-limiting examples of such compounds may include insulin sensitizers with primary action in the liver, insulin sensitizers with primary action in peripheral tissues, insulin secretagogues, compounds that slow the absorption of carbohydrates, and insulin or insulin analogues. Examples of insulin sensitizers with primary action in the liver may include biguanides such as metformin. Examples of insulin sensitizers with primary action in peripheral tissues may include the thiazolidinedione class of drugs, often termed TZDs or glitazones, such as troglitazone, pioglitazone or rosiglitazone. Examples of insulin secretagogues may include sulfonylureas, meglitinides such as repaglinide, or nateglinide. Generally speaking, insulin secretagogues bind to the sulfonylurea receptor (SUR1), a subunit of the ATP-sensitive potassium channel (KATP) on plasma membrane of pancreatic beta cells. Examples of compounds that slow the absorption of carbohydrates may include α-glucosidase inhibitors. Further examples may be found, for instance, in US Patent application no. 2006/0198839, 2006/0079542, 2003/0139429, and 2003/0114469, each of which is hereby incorporated by reference in their entirety.

The amount of a compound capable of modulating insulin secretion comprising a single dosage of the combination will vary depending upon the subject, the compound, and the particular mode of administration. Those skilled in the art will appreciate that dosages may also be determined with guidance from Goodman & Gilman's The Pharmacological Basis of Therapeutics, Ninth Edition (1996), Appendix II, pp. 1707-1711 and from Goodman & Gilman's The Pharmacological Basis of Therapeutics, Tenth Edition (2001), Appendix II, pp. 475-493.

### (c) Compounds Capable of Modulating Weight Gain

In yet another embodiment, the invention encompasses a combination comprising at least one compound capable of modulating weight gain. In some embodiments, the combination comprises at least two, at least three, or at least four compounds capable of modulating weight gain. Methods of detecting and quantifying weight gain are known in the art.

Generally speaking, compounds will include those that decrease body fat or promote weight loss. In one embodiment, acarbose may be administered with any compound described herein. Acarbose is an inhibitor of α-glucosidases and is required to break down carbohydrates into simple sugars within the gastrointestinal tract of the subject. In another embodiment, an appetite suppressant such as an amphetamine or a selective serotonin reuptake inhibitor such as sibutramine may be administered with any compound described herein. In still another embodiment, a lipase inhibitor such as orlistat or an inhibitor of lipid absorption such as Xenical may be administered with any compound described herein. The combination of therapeutic compounds may act synergistically to decrease body fat or promote weight loss.

The amount of a compound capable of modulating weight gain comprising a single dosage of the combination will vary depending upon the subject, the compound, and the particular mode of administration. Those skilled in the art will appreciate that dosages may also be determined with guidance from Goodman & Gilman's The Pharmacological Basis of Therapeutics, Ninth Edition (1996), Appendix II, pp. 1707-1711 and from Goodman & Gilman's The Pharmacological Basis of Therapeutics, Tenth Edition (2001), Appendix II, pp. 475-493.

### (d) Pharmaceutical Combinations

A compound detailed above may be in the form of a free base or pharmaceutically acceptable acid addition salt thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine- (N-methylglucamine) and procaine. All of these salts may be prepared by conventional means from the corresponding compound by reacting, for example, the appropriate acid or base with any of the compounds of the invention.

Combinations of the invention may comprise a pharmaceutical composition. The compounds of the invention may be formulated separately, or in combination. In some embodiments, the compositions may comprise pharmaceutically acceptable excipients. Examples of suitable excipients may include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The compositions may additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention may be formulated so as to provide quick, sustained or delayed release of the active ingredient after administration to a subject by employing procedures known in the art.

The active compounds of the invention may be effective over a wide dosage ranges and are generally administered in pharmaceutically effective amounts. It will be understood, however, that the amount of the compounds actually administered will be determined by a physician, in the light of the relevant circumstances, including the condition to be treated, the analgesic to be administered, the age, weight, and response of the individual patient, the severity of the patient's symptoms, and the like.

Additionally, the compounds may be formulated into pharmaceutical compositions and administered by a number of different means that will deliver a therapeutically effective dose. Such compositions may be administered orally, parenterally, by inhalation spray, rectally, intradermally, transdermally (for instance see US 2006/0084604), or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Solid dosage forms for oral administration may include capsules, tablets, pills, powders, and granules. In such solid dosage forms, the compound is ordinarily combined with one or more adjuvants appropriate to the indicated route of administration. If administered per os, the compound can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

The tablets or capsules of the present invention may be coated or otherwise compounded to provide a dosage form affording the advantage of prolonged action. For example, the tablet or capsule can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate as are known in the art.

The liquid forms in which the compositions of the present invention may be incorporated for administration include aqueous solutions, suitably flavored syrups, oil suspensions and flavored emulsions with edible oils such as cottonseed oil, sesame oil, coconut oil, or peanut oil as well as elixirs and similar pharmaceutical vehicles. Liquid dosage forms for oral administration may also include pharmaceutically acceptable emulsions, solutions, suspensions, and elixirs containing inert diluents commonly used in the art, such as water. Such compositions may also comprise adjuvants, such as wetting agents, emulsifying and suspending agents, and sweetening, flavoring, and perfuming agents.

Injectable preparations of a composition of the invention, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally or intrathecally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

For therapeutic purposes, formulations for administration of the composition may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

### II. Methods

Another aspect of the invention encompasses a method for modulating GIP activity in a subject. The method typically comprises administering a composition to the subject, wherein the composition modulates the xenin activity in the subject. Compounds that modulate xenin activity are detailed in section I(a) above.

Subject, as used herein, may refer to a rodent, a companion animal, a livestock animal, a non-human primate, or a human. Non-limited examples of rodents may include mice, rats, and guinea pigs. Non-limited examples of companion animals include dogs, cats, and horses. Non-limited examples of livestock animals include cattle, goats, and swine.

In certain embodiments, a method for modulating GIP activity in a subject may comprise administering a compound capable of modulating xenin activity in combination with a compound capable of modulating insulin secretion or weight gain as detailed in section 1 above. For such combinations, the compounds may be administered simultaneously, either in the same composition or in more than one composition, or the compounds may be administered sequentially.

As used herein, modulating GIP activity may refer to modulating GIP mRNA concentration, modulating GIP protein concentration, modulating the incretin activity of GIP, and/or modulating GIP's activity in adipocytes. Modulating may refer to increasing or decreasing GIP activity, as discussed in more detail below.

### (a) Modulating the Incretin Activity of GIP

In one embodiment, the invention encompasses a method for modulating the incretin activity of GIP. Generally speaking, the method comprises administering a compound that modulates xenin activity in the subject. For instance, a method for increasing the incretin activity of GIP may comprise administering a compound that increases xenin activity in the subject. Conversely, a method for decreasing the incretin activity of GIP may comprise administering a compound that decreases xenin activity in the subject. Compounds that increase or decrease xenin activity are detailed in section I(a) above.

A method for increasing the incretin activity of GIP may aid in glucose regulation in a subject with type II diabetes or in a subject with impaired glucose tolerance. Consequently, the invention encompasses a method for treating type II diabetes or impaired glucose tolerance. Generally speaking, such a method comprises administering to a subject in need thereof a composition that increases xenin activity in the subject. Increasing xenin activity may in turn increase GIP incretin activity, which in turn may aid in glucose regulation, which may help treat type II diabetes or impaired glucose tolerance. Methods of diagnosing type II diabetes and impaired glucose tolerance are well known in the art. In another embodiment a method for treating type II diabetes or impaired glucose tolerance comprises administering to a subject a combination of compounds detailed in sections I(a) and I(b) above.

### (b) Modulating Activity of GIP in Adipocytes

In another embodiment, the invention encompasses a method for modulating the activity of GIP in adipocytes. Generally speaking, the method comprises administering a compound that modulates xenin activity in the subject. In one embodiment, a method for increasing the activity of GIP in adipocytes may comprise administering a compound that increases xenin activity in the subject. Conversely, a method for decreasing the activity of GIP in adipocytes may comprise administering a compound that decreases the xenin activity in the subject. Compounds that increase or decrease xenin activity are detailed in section I(a) above.

A method for decreasing the activity of GIP in adipocytes may reduce high fat diet-induced obesity in a subject. Consequently, the invention encompasses a method for reducing high fat diet-induced obesity. Methods of diagnosing obesity are known in the art. Generally speaking, a method for reducing high fat diet-induced obesity comprises administering to a subject a composition that decreases xenin activity in the subject. Decreasing xenin activity may in turn decrease GIP activity, which in turn may aid in reducing high fat diet-induced obesity. In another embodiment a method for reducing high fat diet-induced obesity comprises administering to a subject a combination of compounds detailed in sections I(a) and I(c) above that decreases GIP activity in adipocytes.

### (c) Detecting Xenin in a Subject

In certain embodiments, the invention encompasses a method for detecting xenin in a biological sample collected from a subject. Generally speaking, the method comprises collecting a sample from the subject, contacting the sample with an antibody or antibody fragment that specifically recognizes xenin, and detecting the association of the antibody with xenin in the sample. Suitable biological samples may include blood samples, tissue samples, or other suitable biological samples. Methods of collecting blood samples or tissue samples are known in the art.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### DEFINITIONS

As used herein, the phrase “xenin agonist” encompasses xenin receptor agonists.

As used herein, the phrase “xenin antagonist” encompasses xenin receptor antagonists.

As used herein, the phrase “xenin protein” encompasses xenin mimetics.

### EXAMPLES

The following examples illustrate various iterations of the invention.

### Background for Examples 1-7

Enteroendocrine (EE) cells are a complex population of rare, diffusely distributed hormone producing intestinal epithelial cells (1-3). Peptides and hormones secreted by EE cells play important roles in many aspects of gastrointestinal and whole animal physiology (4-6). There are at least 16 different sub-types of EE cells based upon the major product(s) synthesized and secreted by individual cells (1). Several EE cell products including GIP, glucagon-like peptide 1 (GLP-1), ghrelin, cholecystokinin (CCK), and peptide tyrosine tyrosine regulate food intake and/or degree of adiposity (7-11).

GIP is produced predominantly by K cells located in the proximal small intestine and is secreted immediately after ingestion of a meal (4,5,12,13). GIP release is regulated by nutrients in the intestinal lumen, but not by those in the blood (4,6,13,14). Glucose (12,15,16), protein hydrolysates (17), specific amino acids (18), and fat (19) are major GIP secretagogues. Long-term administration of a high fat diet increases intestinal GIP mRNA and peptide levels (12), as well as the circulating amount of plasma GIP (20,21). There is a large body of biochemical and animal data suggesting that GIP signaling promotes the accumulation of fat (22-31). Obese humans also hyper secrete GIP (32-36) suggesting that GIP may promote obesity in humans.

It has long been thought that the major role of GIP was to potentiate glucose-stimulated insulin release from pancreatic islet β-cells. However, mice lacking GIP receptors (GIPR−/−) exhibited only a subtle defect in glucose homeostasis (37) and were protected from the development of obesity and insulin resistance when placed on a high fat diet (21). Furthermore, blood sugar, water intake, hemoglobin A1c, triglyceride, free fatty acid, total cholesterol, LDL cholesterol, and HDL cholesterol levels were not significantly affected by the absence of GIP receptors. These observations could presumably be explained by GLP-1 compensation for the lack of GIPR signaling (38) although additional mechanisms could also contribute. Thus total inhibition of GIPR signaling reduces high fat diet-induced obesity and insulin resistance and is not associated with serious adverse consequences.

Based on the above information it appears that reducing GIP action may have beneficial effects in terms of the development of obesity and insulin resistance. One way to inhibit GIP signaling is to inhibit hormone release from K cells. A potential advantage of this approach is that drugs may be able to target K cells from the intestinal lumen, rather than the blood, thereby avoiding potential side effects associated with systemic delivery of antagonists to either GIP or the GIPR. Results from our laboratory have shown that many of the molecules that regulate GIP release appear to be distinct from those that control hormone release from other types of EE, endocrine, and excitatory cells (39-42). However, the consequences of eliminating or reducing coordinate release of all hormones from K cells are unknown. They may differ from those seen after eliminating the GIPR since K cells have also been reported to produce xenin, a hormone that may promote glucagon release, basal and glucose-stimulated insulin release, secretion from the exocrine pancreas, gut motility, and intestinal microcirculation (43-48). The physiologic importance of xenin or unknown hormones produced by K cells has not been established. The present study was therefore undertaken to define the metabolic consequences of eliminating K cells in mice and in particular to determine whether mice lacking K cells were protected from obesity induced by a high fat diet.

### Materials and Methods for Examples 1-7

Design of Transgenic Constructs—transgenic constructs are illustrated in FIG. 1. GIP/RFP: The λGIP5-2 vector was generously provided by Dr. Rodger Liddle of Duke University (49) and contains the rat GIP promoter as well as a portion of the GIP structural gene. The initiator methionine in exon 2 of the GIP cDNA was converted to an Nco I site and then a Kpn I/Nco I fragment containing 3.1-kb of the rat GIP promoter through this initiator methionine was fused in frame to the initiator methionine for RFP from dsRed 2-1 (Clontech, Mountain View, Calif.). A 1-kb fragment containing the SV40 3′-UTR was PCR amplified from pGL2 basic and cloned downstream of the RFP stop codon. This fragment contains an intron, as well as splicing and polyadenylation signals, to ensure proper processing of the final primary transcript. GIP/DT: The plasmid pIBI30-176 encodes an attenuated diphtheria toxin A chain (DT) and was generously provided by Dr. Ian Maxwell of the University of Colorado Health Science Center (50). An Nco I fragment from GIP/RFP was replaced with the DT cDNA so that DT, rather than RFP, was produced.

Production of Transgenic Mice—GIP/RFP and GIP/DT transgenic mice were produced on a C57BL/6J background through the Washington University School of Medicine Diabetes and Research Training Center Transgenic Core using standard pronuclear injection techniques. Genotyping was conducted on DNA isolated from tail biopsies using PCR and transgene-specific primers. Upstream and downstream primers for the GIP/RFP transgene are 5′-GAG TTC ATG CGC TTC AAG GT-3′ (SEQ ID NO:1) and 5′-CCC ATG GTC TTC TTC TGC AT-3′ (SEQ ID NO:2), respectively. Upstream and downstream primers for the GIP/DT transgene are 5′-CGC CAT GGA TCC TGA TGA TG-3′ (SEQ ID NO:3) and 5′-CCA TGG CTT CAC AAA GAT CGC CTG AC-3′ (SEQ ID NO:4), respectively. Animals were housed in a barrier facility under light-controlled conditions (12-h light and 12-h dark cycle) and given free access to food and water except as indicated for experimental manipulations. Group sizes are indicated in each figure. All experiments in this study were conducted using male mice and animal protocols approved by the Washington University Animal Studies Committee. Statistical analyses were conducted using the student's t-test and/or ANOVA.

Experimental Diets—Animals were continued on standard chow or switched to a high fat diet starting at 8 weeks of age. Standard chow (PicoLab Rodent Diet 20, Ralston Purina, Saint Louis, Mo.) provided 3.08 kcal/g and 11.9% calories from fat. High fat “western” diet (TD.88137, Harlan Teklad, Madison, Wis.) provided 4.5 kcal/g and 42% calories from fat.

Immunohistochemistry—Small intestines were harvested, fixed, sectioned and labeled using indirect immunofluorescence techniques as previously described (40,41). Rabbit polyclonal antibodies to Ds-Red2 were obtained from Clontech. Some animals were injected with bromodeoxyuridine ninety minutes before they were sacrificed in order to label proliferating cells (51). To estimate the number of EE cells that co-express GLP-1 plus GIP, swiss rolls of mouse small intestines from wild type C57BL/6J mice were double-labeled using guinea pig anti-GIP plus rabbit anti-GLP-1 antibodies (41). The number of EE cells positive for GIP alone, GLP-1 alone, or GIP plus GLP-1 in random fields along the entire duodenal to ileal axis were then counted (41,51). Greater than 100 EE cells positive for each incretin were counted in the small intestine of each mouse.

RT-PCR—Procedures were essentially as previously described (39). Briefly, tissues were removed from mice and immediately snap frozen in liquid nitrogen. RNA was isolated from the indicated tissue or segment of the gut and reverse transcribed using the High Capacity cDNA Reverse Transcription Kit (Applied Biosystems, Foster City, Calif.). Aliquots of cDNA were then amplified using the Applied Biosystems 7500 Fast system with the indicated TaqMan gene expression assay and normalized to the amount of beta actin mRNA present in the same sample. Assay numbers are: a) GIP; Mm00433601_m1; b) Chromogranin A (CGA), Mm00514341_m1; c) glucagon, Mm00801712_m1; d) cholecystokinin, Mm00446170_m1; e) somatostatin (SST), Mm00436671_m1; f) ghrelin, Mm00445450_m1; g) secretin, Mm00441235_g1; h) insulin 1, Mm01259683_g1; i) amylin, Mm004394_m1; j) gastrin releasing peptide, Mm00612977_m1; k) gastrin releasing peptide receptor (GRPR), Mm00433860_m1; and l) beta actin, 4352933E. RFP mRNA was assayed using a TaqMan assay custom designed by Applied Biosystems. Forward and reverse primers are 5′-AGC GCG TGA TGA ACT TCG A-3′ (SEQ ID NO:5) and 5′-GCC GAT GAA CTT CAC CTT GTA GAT-3′ (SEQ ID NO:6). Sequence of the FAM-labeled probe was 5′-ACC CAG GAC TCC TCC-3′ (SEQ ID NO:7). Note that tissue-specific processing of preproglucagon generates glucagon-like peptides in intestinal L cells.

Glucose Tolerance Tests—Animals were fasted for 16 h but given free access to water. Blood glucose levels were determined before and at the indicated time after administration of glucose by intragastric gavage (3 mg per g body weight) or by intraperitoneal injection (1 mg per g body weight).

Food Tolerance Tests—Animals were fasted for 16 h but given free access to water. Blood glucose levels were determined before and at the indicated time after animals were given the same type of food that they were previously fed.

Insulin Tolerance Tests—Animals were fasted for 5 h but given free access to water. Blood glucose levels were then determined before and at the indicated times following intraperitoneal injection of recombinant human insulin (0.5 units per kg body weight).

Food Intake—Mice were switched to individualized housing and acclimated for 5 days before measurements were initiated (52). Total daily food intake was averaged over a 4-6 day period. Continuous food intake over a 24 h period was also assessed using the DietMax System (AccuScan Instruments Inc., Columbus, Ohio) at the University of Cincinnati Mouse Metabolic Phenotyping Center.

Intestinal Fat Absorption—Intestinal fat absorption was determined as part of the animals normal feeding regimen using a validated, non-invasive technique that does not require isotope analysis (53). Food and feces analyses were conducted at The University of Cincinnati Mouse Metabolic Phenotyping Center.

Energy Balance—Energy balance was assessed using the PhysioScan Oxygen Consumption/Carbon Dioxide Production System (AccuScan Instruments Inc.) at The University of Cincinnati Mouse Metabolic Phenotyping Center. Mice were placed in the PhysioScan chamber with food 3 h before the dark cycle and energy expenditure was recorded for 24 h. Food was removed the next evening and the animals were fasted for 18 h while additional measurements were recorded.

Assays—Blood glucose concentrations were determined using MediSense Precision Xtra Blood Glucose Test Strips (Abbott Laboratories, Alameda, Calif.). HbA1 c was determined on freshly collected blood using a Bayer Hemoglobin A1c reagent kit with the DCA 2000+ Analyzer according to the manufacturer's instructions (Bayer HealthCare LLC, Elkhart, Ind.). For determination of GIP, insulin, glucagon, and leptin, blood was added to chilled tubes. Plasma was then prepared and assayed for total GIP or insulin using ELISAs (Linco Research, Inc., Saint Charles, Mo.). Leptin, glucagon, and amylin, were assayed using a LincoPlex assay (Linco Research, Inc.). Active GLP-1 was measured in plasma containing a DPP IV inhibitor (Linco Research, Inc) using an ELISA according to the manufacturer's protocol (Linco Research, Inc). Since levels of active GLP-1 are very low following oral administration of 3 mg glucose per g body weight, mice were orally administered a high dose of glucose (6 mg/g body weight) (54) or 3 mg/g glucose plus intralipid (55) when this hormone was to be measured.

Nuclear Magnetic Resonance Imaging (MRI)—Conscious mice were placed in a restraint tube and analyzed using an EchoMRl (EchoMedical Systems, Houston, Tex.) to estimate lean body mass, fat tissue mass, and water composition.

Dual-Energy X-Ray Absorptionmetry (DEXA—DEXA was conducted on anesthetized mice using a small animal densitometer (Lunar PIXImus, Madison, Wis.).

### Example 1

### Regulatory Eements from the GIP Promoter and Gene Confine Transgene Expression to GIP-Producing Cells In Vivo

DT-mediated ablation of GIP-producing cells requires DNA regulatory elements that confer proper transgene expression. It was previously reported that 2.5-kb of the rat GIP promoter drives human insulin transgene expression specifically in K cells of transgenic mice (56). We generated multiple lines of transgenic mice using a similar construct and noted inappropriately high levels of human preproinsulin transcripts in the stomach of transgenic mice. Thus, a transgene containing additional regulatory sequences from the rat GIP promoter and gene was prepared (FIG. 1) and then used to drive RFP expression in transgenic mice. Real time PCR using RNA prepared from multiple tissues revealed that relative levels of endogenous GIP transcripts and transgene encoded RFP mRNAs are very tightly correlated. Furthermore, detectable levels of both gene products were observed only in the gut of GIP/RFP animals with highest levels in the proximal small intestine (FIG. 2). Immunohistochemical analyses revealed that within the intestine, RFP expression is confined to GIP-producing cells (FIG. 3). Taken together, these results indicate that these regulatory elements from the rat GIP promoter and gene target reporters to the appropriate cells in vivo.

### Example 2

### The GIP/DT Transgene Ablates Only GIP-Producing Cells

Forced expression of an attenuated Diphtheria Toxin A chain [DT; (50)] is a well-established strategy to ablate specific cell lineages in transgenic mice (57-59). It is important to note that this mutant DT exhibits greatly reduced toxicity compared to the wild type toxin which eliminates killing of cells adjacent to those targeted by the transgene as well as those that may exhibit very low levels of “leaky” promoter activity. This particular attenuated DT has been used to specifically ablate Paneth cells (60) and goblet cells (61) in the intestinal epithelium without killing adjacent cells or eliciting an immune response.

Transgenic mice were generated that express the GIP/DT transgene (FIG. 1). Histochemical staining of intestines using hematoxylin and eosin, alcian blue, periodate acid Schiff, and phloxine/tartrazine (51) demonstrate that cellular morphology, crypt-villus architecture, and Paneth and goblet cell numbers are similar in wild type and GIP/DT mice. The number of bromodeoxyuridine-labeled intestinal epithelial cells was also similar in both wild type and GIP/DT transgenic animals. Furthermore, bromodeoxyuridine-positive cells were confined to the mid-portion of the intestinal crypts. Thus, DT expression did not perturb cell proliferation. EE cell-derived hormones are each expressed in unique patterns along the proximal to distal axis of the gut [(3-6) see also FIG. 4]. RNA was isolated from the stomach and sequential segments of the small intestines from wild type and GIP/DT mice that had been fed standard chow. Liver served as a negative control. Real time PCR assays were utilized to quantify transcript levels. GIP mRNA levels in wild type mice is extremely high in the proximal small intestine and very low in the stomach and distal small intestine (FIG. 4B). Transcripts encoding GIP are greatly reduced in the intestines from GIP/DT animals. In the mouse, CGA is produced by many types of EE cells but not by those that produce GIP (41). CGA transcripts were present at similar levels along the entire proximal to distal axis of intestines from wild type and GIP/DT animals (FIG. 4C). Immunohistochemical studies confirm that GIP is present in singly dispersed EE cells in the proximal small intestine of wild type mice but is undetectable in intestines from GIP/DT animals. In contrast, CGA is present in normal numbers of EE cells in the small intestine of both wild type and GIP/DT mice. Consistent with reduced GIP mRNA levels and GIP-producing cells, circulating GIP is detectable in plasma prepared from wild type (86+/−26 pg/ml), but not GIP/DT (<3.3 pg/ml) mice. To confirm that GIP action is abolished, the incretin response was measured in mice that had been maintained on standard chow. Fifteen minutes after administration of oral glucose (3 mg/g body weight) to fasted animals, plasma insulin levels increased 5-fold in wild type mice but remained unchanged in the GIP/DT animals (FIG. 5A). Essentially identical results were obtained when mice were orally administered twice the dose of glucose or glucose plus intralipid (Table 2). Amylin is co-released with insulin from β-cells. Oral glucose-stimulated amylin release was also abolished in the GIP/DT mice fed standard chow. Thus, the absence of GIP-producing cells results in the complete loss of an incretin response (see below). Fasting plasma glucagon levels were similar in wild type and GIP/DT mice and decreased comparably in response to oral glucose (FIG. 5B).

TABLE 2
Plasma GLP-1 levels are similar in wild type and GIP/DT mice. Wild type (WT) and GIP/DT mice
(DT) maintained on standard chow (Chow) or high fat (HF) food were fasted for 16 h. Blood was
then collected before (fasting) or 15 minutes after oral administration of glucose (Glc; 6 mg/g
body weight) or glucose plus intralipid (Glc/Lipid; 3 mg glucose plus 4.5 μL 20% intralipid
per gram body weight). Plasma was assayed for active GLP-1 or insulin by ELISA. Note that plasma
GLP-1 levels in the GIP/DT mice are similar to or higher than those in nearly all groups of
similarly treated wild type mice and high fat feeding results in elevated GLP-1 levels. Aln
these groups, GLP-1 levels were below the limits of detection in 5 out of 12 wild type and 3
out of 10 GIP/DT animals. Thus, values representing the lowest limit of detectable GLP-1 in
the assay were used to estimate maximum average GLP-1 levels for these animals.

| | GLP-1 (pM) | | | Insulin (ng/ml) | | |
|---|---|---|---|---|---|---|
| Treatment | Fasting | Glc | Glc/Lipid | Fasting | Glc | Glc/Lipid |
| WT/Chow | ≦0.9^{A} | 2.4 +/− 0.5 | 2.0 +/− 0.3 | 0.40 +/− 0.07 | 1.47 +/− 0.20 | 1.41 +/− 0.24 |
| (n = 12) | | | | | | |
| DT/Chow | ≦1.5^{A} | 2.7 +/− 0.5 | 3.5 +/− 0.5 | 0.33 +/− 0.07 | 0.43 +/− 0.04 | 0.47 +/− 0.21 |
| (n = 10) | | | | | | |
| WT/HF | 3.1 +/− 0.7 | 11 +/− 1.6 | 7.5 +/− 3.7 | Not Done | Not Done | Not Done |
| (n = 4-9) | | | | | | |
| DT/HF | 3.6 +/− 0.7 | 5.5 +/− 0.9 | 6.7 +/− 3.2 | Not Done | Not Done | Not Done |
| (n = 3-8) | | | | | | |

The complete lack of an incretin response raised the possibility that the GIP/DT transgene also ablated GLP-1-producing cells. K cells do not co-express CCK, SST, substance P, serotonin, gastrin, or secretin (1,4-6,62). In contrast, a subset of EE cells in humans and pigs have been reported to produce both immunoreactive GIP and GLP-1 (63,64). However, less than 3% of the EE cells in the mouse intestine were reported to co-express GIP plus GLP-1 (1). To confirm this latter observation, paraffin embedded sections of intestines from wild type C57BL/6J mice were stained for GIP and GLP-1. Consistent with the published data from an independent lab (1), co-staining for both incretins was observed in only 2.3%+/−0.2% of the EE cells. GLP-1 is produced by cell-specific processing of preproglucagon. As shown in FIG. 4D, there are no statistically significant differences in preproglucagon mRNA levels in the stomach or small intestine in wild type versus GIP/DT mice. Similar numbers of GLP-1-immunoreactive cells were also observed in the small intestines from wild type and GIP/DT mice. Next, GLP-1 release was measured before and fifteen minutes after administration of oral nutrients. Fasting GLP-1 levels hovered around the lower limits of detection in wild type and GIP/DT mice fed standard chow (Table 2). Fifteen minutes after administration of oral glucose or glucose plus intralipid to fasted mice, plasma GLP-1 increased to similar levels in wild type and GIP/DT animals (Table 2). Gastrin releasing peptide (GRP) is produced by enteric neurons and is important for promoting oral glucose-stimulated GLP-1 release (54). The mRNA levels for GRP and its receptor are similar in intestinal samples from wild type and GIP/DT mice (FIGS. 4E and F). GLP-1 action is also normal since intraperitoneal administration of GLP-1 along with glucose improved glucose excursion from blood to similar extents in wild type and GIP/DT mice (FIG. 6). Taken together, these observations indicate that GLP-1 production, release, and action are apparently normal in GIP/DT mice fed standard chow. Transcripts encoding SST, CCK, secretin, and ghrelin were also similar in the stomach and nearly all intestinal segments from wild type and GIP/DT mice (FIGS. 4G to 4J).

### Example 3

### Mice Lacking GIP-Producing Cells Show Attenuated Weight Gain on a High Fat Diet

Wild type and GIP/DT mice were each randomized to 2 groups at 8 weeks of age. One group for each genotype (n=7-9 mice per group) was switched to a high fat “western” diet and the other group was maintained on standard chow. As shown in FIG. 7, age-matched wild type and GIP/DT mice maintained on standard chow exhibit similar body weights (30.2+/−0.7 g versus 29.8+/−1.0 g, respectively, at 35 weeks p=0.74). Wild type and GIP/DT mice maintained on the high fat diet for 35 weeks weighed 55% (p<0.001) and 11% (p<0.01), respectively, more than those maintained on standard chow. Thus, on a high fat diet, GIP/DT mice gained five times less weight than wild type littermates. Circulating leptin levels correlate well with body fat. Consistent with the body weight data, plasma leptin levels were similar in wild type and GIP/DT mice fed standard chow (FIG. 5C). Following high fat feeding for 15-weeks, leptin levels increased nearly 20-fold in wild type mice. This increase was markedly attenuated in the GIP/DT animals. MRI and DEXA analyses confirmed that the GIP/DT mice fed a high fat diet have decreased fat mass when compared to similarly fed wild type animals. In contrast, lean and fat body masses were similar in aged-matched wild type and GIP/DT mice that were maintained on standard chow. Thus animals lacking GIP-producing cells resist development of high fat diet-induced obesity.

### Example 4

### Mice Lacking GIP-Producing Cells Exhibit Decreased Intake of High Fat Food and Increased Energy Expenditure

Wild type and GIP/DT mice maintained on standard chow consumed similar amounts of food per day (FIG. 8). Conversely, GIP/DT mice maintained on a high fat diet for 21-weeks consumed 16% less high fat food per day than did age-matched wild type control animals. The pattern of food consumption over a 24-h period was similar in both groups of mice. The percentage of available fat that was absorbed from the high fat food was similar in both the GIP/DT and wild type mice fed the high fat diet (99.4+/−0.1 versus 98.7+/−0.4, respectively; p=0.15). As shown in FIG. 9, oxygen consumption, carbon dioxide production, and heat output were increased in GIP/DT mice fed the high fat diet. This increased energy expenditure was noted only during the dark cycle of fed animals. In contrast, the respiratory quotient was similar in both lines of mice. Taken together, these results suggest that decreased food intake coupled with increased energy expenditure accounts for the reduced body weight of the GIP/DT mice fed a high fat diet.

### Example 5

### Mice Lacking GIP-Producing Cells do not Develop Insulin Resistance on a High Fat Diet

Insulin sensitivity was assessed to determine if the reduced weight gain in the GIP/DT mice was associated with changes in insulin action. Following intraperitoneal administration of insulin (0.5 units per kg), glucose excursion from blood is essentially identical in wild type and GIP/DT mice maintained on standard chow for up to 33 weeks (FIG. 10A). Following 9 weeks on a high fat diet, the wild type mice exhibit a modest reduction in the glucose response to insulin when compared to GIP/DT animals (15% reduction in the area under the curve, p<0.01). After 33 weeks of high fat feeding, glucose excursion from blood is markedly improved in the GIP/DT mice compared to wild type animals (FIG. 10B). As an additional measure of insulin sensitivity, insulin to glucose ratios were determined in mice fed standard chow or high fat diets for 27-weeks. Random blood glucose levels are similar in all mice regardless of genotype or diet (FIG. 10C). However, the plasma insulin level, and thus, the insulin to glucose ratio, is elevated only in wild type animals maintained on the high fat diet (FIGS. 10D and E). Similar insulin to glucose ratios are observed following 5- and 16 h fasts. Thus, the GIP/DT mice do not develop high fat diet-induced insulin resistance.

### Example 6

### Glucose Homeostasis Following Oral and Intraperitoneal Glucose Administration is Nearly Normal in Mice Lacking GIP-Producing Cells

Since GIP potentiates insulin secretion in response to oral nutrients, glucose homeostasis was assessed in the GIP/DT mice. On each diet, glucose excursion from blood is similar in wild type and GIP/DT animals following intraperitoneal administration of glucose (FIG. 11A, B). This is consistent with data indicating that insulin and amylin mRNA levels are normal in the pancreas of GIP/DT mice (FIG. 4). However, regardless of genotype, animals fed the high fat diet exhibited reduced glucose clearance compared to mice fed standard chow. Importantly, glucose excursion did not worsen with age in the GIP/DT mice on either diet. In mice fed standard chow, plasma insulin levels both before and 2 minutes after intraperitoneal injection of glucose were essentially identical suggesting β-cell function is not perturbed in the GIP/DT mice (FIG. 12). Forty five minutes after the glucose injection, plasma insulin levels were lower in the GIP/DT versus wild type mice (p<0.05). Since both of these groups exhibit similar glucose clearance from blood following intraperitoneal injection of glucose (FIG. 11A), the GIP/DT animals may have improved insulin sensitivity. In mice fed the high fat diet, plasma insulin levels are increased 45 minutes, but not 2 minutes, after intraperitoneal administration of glucose, regardless of genotype (FIG. 11A). Thus, high fat feeding results in delayed insulin release but this effect is independent of the presence/absence of GIP-producing cells.

Blood glucose excursion in response to administration of oral glucose was also assessed in the same animals (FIG. 11C, D). As expected for mice lacking an incretin effect, the GIP/DT mice fed standard chow exhibit a reduced rate of blood glucose clearance compared to similarly fed wild type animals. Following high fat feeding for 31-weeks, the blood glucose excursion rate is also reduced in the GIP/DT versus wild type animals but is not worse than that in the GIP/DT mice fed standard chow. Unlike the case on standard chow, the GIP/DT mice fed a high fat diet exhibit a modest increase in oral glucose-stimulated insulin release (FIG. 5A). Fasting plasma GLP-1 levels are similar in both wild type and GIP/DT fed the high fat diet and are higher than those observed in the mice fed standard chow (Table 2). However, following administration of oral nutrients, plasma GLP-1 levels were greater in the wild type versus GIP/DT mice (Table 2). This suggests that on a high fat diet, the partially restored incretin effect may not be due to increased GLP-1 release.

### Example 7

### Glucose Homeostasis Following Ingestion of Physiologic Meals is Nearly Normal in Mice Lacking GIP-Producing Cells

Blood glucose excursion rates in response to administration of a single, high dose, oral glucose load may not reflect the response to ingestion of a mixed meal. Therefore, blood glucose levels were measured before and after fasted animals were given free access to the same type of chow on which they had been previously maintained (FIGS. 11E and F). Although blood glucose excursion rates are reduced in the GIP/DT versus wild type mice on either diet, blood glucose levels never exceed 275 mg/dL in the GIP/DT animals on either diet. To assess long-term glucose homeostasis, HbA1 c levels in blood were determined in animals that had been maintained on standard chow or high fat diets for 36 weeks. On standard chow, HbA1 c levels of 3.7% were observed for both wild type and GIP/DT mice. Following 36 weeks of high fat feeding, HbA1 c levels were increased to 4.2% (p=0.001) in the GIP/DT mice (FIG. 13). Thus, long-term glucose homeostasis is only slightly perturbed in these animals. Furthermore, the decreased body weight in the GIP/DT mice fed a high fat diet is not due to diabetes.

### Discussion for Examples 1-7

Identification of K cell-specific regulatory elements. In the mouse, highest levels of GIP transcripts are present in the proximal small intestine and much lower levels are expressed in the stomach and distal intestine (FIGS. 2 and 4). It was previously reported that 2.5-kb of the rat GIP promoter fused to the human insulin gene confers tissue-specific and K cell-specific insulin expression in transgenic mice (56). However, FIG. 2A from this same report revealed that highest levels of transgene-encoded insulin transcripts were observed in the stomach, not the duodenum, of the transgenic mice. Using a similar transgene, this same pattern of mis-expression was observed in independently generated transgenic mice. Therefore, additional regulatory elements required for proper GIP gene expression are located outside of this 2.5-kb promoter fragment. A new transgene (FIG. 1) was prepared that incorporated 3.1-kb, rather than 2.5-kb, of the rat GIP promoter. Since sequence analysis identified potential regulatory elements within intron 1 of the GIP structural gene (49), this entire intron was also included in the new transgene. Using RFP as a reporter, these additional regulatory elements conferred appropriate tissue-, region-, and cell-specific expression in vivo (FIGS. 2 and 3). The fidelity of transgene expression was further illustrated by the fact that only transcripts encoding GIP were ablated along the stomach to ileum axis of GIP/DT mice. In contrast to a previous report using rats (65), there was no evidence for GIP expression in the submandibular salivary gland of mice since neither GIP nor RFP transcripts were detected by our highly sensitive and specific real time PCR assays in this tissue in GIP/RFP animals. This is the first report describing a transgene that confers appropriate reporter expression to GIP-producing cells in transgenic mice. Although it is not known whether the additional critical regulatory elements reside within intron 1 or the additional 600-bp of the promoter, it is interesting to note that sequences located within intron 1 of the glucagon gene are also required for proper transgene expression in transgenic mice (66). Due to the importance of K cell-derived hormones for whole animal physiology, these novel regulatory elements will be extremely useful for generating additional transgenic mice that can be used to probe K cell function in vivo.

Ablation of GIP-producing cells eliminates the incretin response to oral glucose. Oral glucose-stimulated insulin release was essentially eliminated in the GIP/DT mice fed standard chow (FIG. 5A and Table 2) indicating that the total incretin response was abolished by ablating K cells. This observation was quite surprising since GIPR−/− mice exhibited less than a 2-fold reduction in oral glucose-stimulated insulin release (37,67,68). In fact, 15-minutes after administration of oral glucose (3 mg/g body weight) to male GIPR plus GLP-1 R double knockout mice, plasma insulin levels still increased nearly 5-fold (68). This incretin effect is only slightly reduced compared to that observed in the single GIPR or GLP-1 R mice. In contrast, insulin release increased only 30-50% in the GIP/DT mice following administration of oral glucose (FIG. 5A). The absence of oral glucose-stimulated insulin release in GIP/DT mice fed standard chow does not result from a β-cell insufficiency since glucose excursion from blood in response to intraperitoneal glucose is normal in these animals (FIG. 11A). Furthermore, insulin and amylin mRNA levels are similar in pancreatic RNA samples prepared from wild type and GIP/DT mice (FIG. 4). Importantly, GLP-1 production, release, and action were not attenuated in the GIP/DT mice fed standard chow (FIGS. 4 and 6, Table 2). Taken together, these observations raise the possibility that GIP/DT mice are lacking not only GIP, but also another major K cell-derived incretin(s) that is released following ingestion of nutrients.

Xenin has been reported to be produced by a sub-population of K cells (43). It is important to note that this hormone is a cleavage product derived from the ubiquitously expressed alpha subunit of the coat protein (69) and thus, could potentially arise from non-physiologic proteolysis of this protein. Supraphysiologic concentrations of xenin increased glucose-stimulated insulin release in perfused rat pancreas (44). In contrast to GIP, xenin is also released in response to sham feeding (70). Clearly, the physiologic importance for xenin has not been established.

Ablation of GIP-producing cells reduces diet-induced obesity and insulin resistance. Numerous physiologic studies strongly suggested that GIP plays an important role in promoting high fat diet-induced obesity and insulin resistance. Studies using GIPR−/− mice provided genetic evidence in support of this hypothesis. Biochemical studies suggest that GIP promotes weight gain by increasing glucose uptake, heparin-releasable lipoprotein lipase activity, and fat storage by adipocytes (21). Two potential strategies to reduce GIP signaling, and thus obesity and insulin resistance, in vivo would be to 1) inhibit GIPR activity by administration of GIPR antagonists and 2) inhibit GIP production, release, and/or action. Results presented in this paper indicate that as in GIPR−/− mice, animals genetically engineered to lack GIP-producing cells also resist development of high fat diet-induced obesity and insulin resistance. Importantly, the complete absence of GIP plus K cell-derived xenin or other unknown hormones does not appear to result in serious adverse effects. Furthermore, in both model systems, elimination of the GIP-mediated incretin effect did not lead to severely impaired glucose homeostasis since HbA1 c levels were similar in wild type and GIP/DT mice regardless of the diet (FIG. 13). This is particularly noteworthy since the GIP/DT mice do not have a demonstrable incretin effect when maintained on a standard chow diet. Taken together, these observations suggest reducing hormone production and release by K cells is a potential strategy to prevent and/or ameliorate diet-induced obesity and insulin resistance.

Common, as well as distinct, biochemical and physiological pathways appear to be perturbed in GIPR−/− and GIP/DT mice. Loss of GIP signaling presumably explains the amelioration of obesity and insulin sensitivity in both model systems. The fact that the GIP/DT mice consume less high fat food per day and exhibit greater energy expenditure than similarly fed wild type mice can most probably account for their reduced weight gain (FIGS. 7-9). Although there are conflicting reports concerning high fat food intake in GIPR−/− versus wild type mice (37,67), the observations reported herein agree with those reported for long-term diet related studies (67).

### Example 8

### Role of Xenin in Insulin Regulation

Transgenic Mice That do not Contain Intestinal K Cells—the cells that make GIP-have been generated. Unlike mice lacking GLP-1 plus GIP signaling, the GIP/DT mice do not exhibit a significant incretin response (FIG. 14) indicating that K cells produce either a novel incretin or a hormone required for the incretin response.

K cells produce a hormone in addition to GIP called xenin. The physiologic importance of xenin is unknown. Experiments using purified islets (FIGS. 15, 16) and insulin producing cell lines (FIG. 15) indicate that unlike GIP and GLP-1, the two known classical incretin hormones, xenin does not potentiate glucose-stimulated insulin release in vitro. Furthermore, unlike GIP and GLP-1, xenin alone fails to increase the rate of blood glucose clearance in wild type mice (FIG. 17). These results indicate that xenin does not exhibit classical incretin-like activity.

In vivo experiments indicate that GLP-1 exhibits classical incretin-like activity in both wild type and GIP/DT mice since GLP-1 potentiates the rate of blood glucose clearance similarly in both lines of mice (FIG. 17). Thus, the incretin activity of GLP-1 does not require products released from K cells. However, GIP exhibited incretin-like activity in wild type, but not GIP/DT, mice (FIG. 17) suggesting that the GIP-mediated incretin response requires additional hormones produced by K cells.

GLP-1, GIP and xenin are all released from enteroendocrine cells immediately after ingestion of nutrients. The GIP/DT mice do not contain K cells, and thus, are lacking (or contain reduced levels of) GIP and xenin. In contrast to GIP or xenin alone, a combination of GIP plus xenin restores the incretin effect in GIP/DT mice (FIG. 17).

A xenin receptor has not yet been identified. The C-terminus of xenin is highly homologous to that of neurotensin (NT) and it is thought that xenin may signal by binding to NT receptors. However, NT only partially restored the GIP-mediated incretin effect in GIP/DT mice since blood glucose clearance rates did not increase to the same extent as was observed with wild type animals [(FIG. 17); although note that GIP alone is sufficient for the GIP-mediated incretin response in wild type mice]. Therefore, NT and xenin exhibit overlapping, but not identical, effects on the GIP-mediated incretin response.

Since NT and/or xenin could potentially co-operate with GIP to elicit the GIP-mediated incretin response, we determined whether NT mRNA levels are similar in the intestines from wild type and GIP/DT mice. As shown in FIG. 18, NT mRNA levels increase progressively and to similar levels from the stomach to ileum of both wild type and GIP/DT mice. Since GIP/DT mice lack K cell-derived xenin but do not lack NT, the GIP-mediated incretin effect in the GIP/DT mice most likely requires xenin rather than NT.

GLP-1 release in response to oral glucose is similar in WT and GIP/DT animals (Table 3). Thus if xenin is involved in the endogenous GLP-1 mediated incretin response, intraperitoneal injection of xenin along with administration of glucose by intragastric gavage should increase blood glucose clearance rates in GIP/DT mice. As shown in FIG. 19, xenin had no effect on blood glucose clearance following administration of oral glucose. Thus, xenin potentiates the incretin effect mediated by GIP, but not GLP-1.

TABLE 3
GLP-1 (pM) Insulin (ng/ml)
Plasma GLP-1 levels are similar in wild type and GIP/DT mice. Wild type (WT) and GIP/DT mice (DT)
maintained on standard chow were fasted for 16-h. Blood was then collected before (fasting) or
15 minutes after oral administration of glucose (Glc; 6 mg/g body weight) or glucose plus intralipid
(Glc/Lipid; 3 mg glucose plus 4.5 μL 20% intralipid per gram body weight). Plasma was
assayed for active GLP-1 or insulin by ELISA. Note that plasma GLP-1 levels in the GIP/DT mice are
similar to or higher than those in similarly treated wild type mice. Aln these groups, GLP-1 levels
were below the limits of detection in 5 out of 12 wild type and 3 out of 10 GIP/DT animals.
Thus, values representing the lowest limit of detectable GLP-1 in the assay were used to
estimate maximum average GLP-1 levels for these animals.

| Treatment | Fasting | Glc | Glc/Lipid | Fasting | Glc | Glc/Lipid |
|---|---|---|---|---|---|---|
| WT/Chow | ≦0.9A | 2.4 +/− 0.5 | 2.0 +/− 0.3 | 0.40 +/− 0.07 | 1.47 +/− 0.20 | 1.41 +/− 0.24 |
| (n = 12) | | | | | | |
| DT/Chow | ≦1.5A | 2.7 +/− 0.5 | 3.5 +/− 0.5 | 0.33 +/− 0.07 | 0.43 +/− 0.04 | 0.47 +/− 0.21 |
| (n = 10) | | | | | | |

These results suggest for the first time that the failure of GIP to increase insulin secretion in the setting of glucose intolerance or diabetes may be related to a concomitant deficiency of the gastrointestinal peptide xenin. Xenin, or agents that increase xenin signaling, either alone or in combination with GIP, other intestinal peptides such as GLP-1 or more conventional treatments of diabetes, may thus be important therapeutic agents for safely and effectively treating glucose intolerance or diabetes.

Additionally, since GIP plays an important role in promoting obesity and xenin increases GIP action in islet b-cells, reducing xenin, or agents that reduce xenin signaling, either alone or in combination with GIP, other intestinal peptides such as GLP-1 or more conventional treatments of obesity, may thus be important therapeutic agents for safely and effectively treating obesity.

### Example 9

### Xenin-25 Potentiates GIP-Mediated Insulin Release in GIP/DT Mice

In Example 8, data demonstrated that GIP plus Xenin-25, but not GIP alone, reduces hyperglycemia in GIP/DT mice following the intraperitoneal injection of glucose. In this example, it is demonstrated that the reduction in hyperglycemia in GIP/DT mice is preceded by increased insulin release. Thus, Xenin-25 improves hyperglycemia by potentiating GIP-mediated insulin release.

WT and DT mice were fasted for 16-h (Fast). As indicated, some mice were then administered an intraperitoneal injection of glucose (1 g/kg) with vehicle alone (BSA), GIP alone, Xenin-25 alone (Xen) or GIP plus Xenin-25 (G+X). Five minutes later, blood was collected. Plasma was then prepared and assayed for insulin. In WT animals, injection of GIP alone results in a greater than 3-fold increase in plasma insulin levels and Xenin-25 does not significantly increase this GIP-mediated insulin release (G+X; FIG. 20). In contrast, injection of GIP in DT mice 1) elicited a much smaller increase in plasma insulin levels than was observed in WT mice and 2) injection of GIP plus Xenin-25 (G+X) increased plasma insulin levels to the same level noted in WT mice receiving GIP alone. Xenin-25 alone failed to increase insulin levels in either WT or DT mice.

### Example 10

### Xenin-25 Potentiates GIP Action in a Mouse Model of Human T2DM

Like humans with type 2 diabetes mellitus [T2DM; (71-73)], GIP/DT mice exhibit an incretin response to exogenously administered GLP-1, but not GIP (FIG. 17). Experiments here demonstrate that Xenin-25 also potentiates a GIP-mediated reduction in hyperglycemia in a mouse model of human T2DM.

NONcNZO10/Ltj mice exhibit a spontaneous, progressive, polygenic form of T2DM that is similar to that observed in humans (74;75). At the age indicated in FIG. 21, mice were fasted for 16-h. Blood glucose levels were measured before and at the indicated time after an intraperitoneal injection of glucose (1 g/kg) plus vehicle alone (BSA), GIP alone, Xenin-25 alone (Xen) or GIP plus Xenin-25 (GIP+Xen). In response to the intraperitoneal injection of glucose: 1) control as well as treated mice exhibit rapid reductions in hyperglycemia; 2) GIP alone does not significantly improve blood glucose levels 3) Xenin-25 potentiates the GIP-mediated reduction in blood glucose; and 4) Xenin-25 alone has no impact on blood glucose levels (FIG. 21A). In response to the intraperitoneal injection of glucose: 1) blood glucose levels in control mice (BSA) are much higher than similarly treated 5-week old control animals; 2) GIP alone causes a modest improvement in blood glucose levels; 3) Xenin-25 potentiates the GIP-mediated reduction in blood glucose levels to a much greater extent than is observed with 5-week old animals; and 4) Xenin-25 alone has no effect on blood glucose levels (FIG. 21B). Regardless of age, GIP plus Xenin-25 reduces hyperglycemia to a much greater extent than does GIP alone (FIG. 21C).

### Example 11

### Xenin-25 Potentiates GIP-Mediated Insulin Release in a Mouse Model of Human T2DM

The previous experiments demonstrated that Xenin-25 potentiates a GIP-mediated reduction in hyperglycemia in a mouse model of human T2DM. Experiments here demonstrate that Xenin-25 improves the GIP-mediated reduction in hyperglycemia in the NONcNZO10/Ltj mice by potentiating GIP-mediated insulin release.

NONcNZO10/Ltj mice were fasted for 16-h. Blood was collected before (0) or 15 minutes after an intraperitoneal injection of glucose (1 g/kg) in the presence of vehicle alone (BSA), GIP alone, Xenin-25 alone (Xen) or GIP plus Xenin-25 (GIP+Xen). GIP alone or Xenin-25 alone has little effect on plasma insulin levels following the intraperitoneal injection of glucose [compared to mice receiving vehicle alone (BSA)] (FIG. 22A). In contrast, Xenin-25 potentiated the GIP-mediated increase in plasma insulin levels. Results presented in FIG. 22B shows that: 1) fasting insulin levels (0 minutes) are higher in the 18-week old mice compared to 5-week old animals and 2) insulin release in response to Xenin-25 plus GIP is much greater than that in response to GIP alone.

Data presented in Examples 9-11 indicate that:

1. Xenin-25 potentiates GIP-mediated insulin release in GIP/DT mice

2. Xenin-25 potentiates the GIP-mediated reduction in hyperglycemia following an intraperitoneal injection of glucose in mice with diabetes.

3. Xenin-25 potentiates GIP-mediated insulin release following an intraperitoneal injection of glucose in mice with diabetes.

4. Xenin-25 alone has no impact on blood glucose or plasma insulin levels in either GIP/DT or NONcNZO10/Ltj mice

### Example 12

### Xenin-25 Does Not Directly Activate Islet Beta Cells

Experiments were conducted to determine whether Xenin-25 acts directly on pancreatic islet beta cells (FIG. 23). Islets were isolated from wild type C57BL/6J mice and then subjected to insulin release assays (FIG. 23A) in the presence of different concentrations of glucose. In the presence of stimulating, but not basal, concentrations of glucose, insulin release is potentiated by inclusion of either GLP-1 or GIP in the assay buffer. Thus, the purified islets respond appropriately to classical incretin hormones. In contrast to GLP-1 or GIP, Xenin-25 exerted no effect on glucose-stimulated insulin release in the isolated islets. A dose-response curve for Xenin-25 revealed that the lack of the incretin response was not due to the presence of inhibitory concentrations of the peptide (Not Shown).

Next, studies were performed with MIN6 cells, a well-characterized, glucose-responsive, insulin-producing cell line. First, insulin release assays were conducted in the presence of peptides. As with primary mouse islets, glucose-stimulated insulin release was amplified by addition of GLP-1 or GIP, but not by Xenin-8 (Not Shown) or Xenin-25 (FIG. 23B). Second, Min6 cells were treated with GLP-1, GIP, or Xenin-25 and Western blots conducted to determine whether the Xenin-25 increased MAPK signaling. As shown in FIG. 24, both GLP-1 and GIP caused profound increases in phosphorylation of MAPK whereas Xenin-25 had little effect. In contrast to results with Min6 cells, Xenin-25 greatly increased phosphorylation of MAPK in Panc1 cells—a human exocrine pancreas adenocarcinoma cell line (FIG. 24). Finally, insulin release assays were conducted in the presence of GIP plus Xenin-25. As shown in FIG. 23, GIP caused a dose-dependent increase in insulin release which was unaffected by the presence of Xenin-25. Taken together, these experiments suggest that Xenin-25 indirectly increases insulin release from pancreatic islet b-cells in vivo.

### Example 13

### Cholinergic Neurons Relay the Xenin-25 Signal to Beta Cells In Vivo

Activation of parasympathetic neurons that innervate the pancreas can increase glucose-mediated insulin release in vivo. Since Xenin-25 does not act directly on islet beta cells, studies were conducted to determine whether products released from parasympathetic neurons could relay the Xenin-25 signal to beta cells. Wild type and GIP/DT mice were fasted overnight and then administered glucose in combination with GIP plus Xenin-25 by intraperitoneal injection. Animals also received an intraperitoneal injection of atropine or saline 15 minutes before the glucose. Blood was collected and plasma prepared 5 minutes after the glucose injection. As shown in FIG. 25A, atropine, a competitive antagonist of muscarinic acetylcholine receptors, inhibited the increase in plasma insulin level 1.8-fold in GIP/DT mice (P=0.04). The magnitude of this reduction mirrors the 1.6-fold increase in plasma insulin levels in GIP/DT mice receiving GIP plus Xenin-25 versus GIP alone (see FIG. 20). Atropine had no statistically significant effect on insulin release from the wild type mice (FIG. 25A). Likewise, Xenin-25 had little effect on plasma insulin levels in wild type mice that received GIP plus Xenin-25 versus GIP alone (FIG. 20). Atropine had no effect on plasma insulin levels in either wild type or GIP/DT mice when GIP was administered in the absence of Xenin-25 (FIG. 25B). Thus, atropine inhibits only the Xenin-25 component of the GIP plus Xenin-25-mediated increase in plasma insulin. Subsets of parasympathetic neurons that innervate the islets release vasoactive intestinal polypeptide (VIP) and pituitary adenylate cyclase activating peptide (PACAP) when activated. N-terminally truncated forms of PACAP [PACAP(6-38) or PACAP(6-27)] are competitive inhibitors of both VIP receptors (VPAC1 and VPAC2) as well as the PACAP-specific receptor Pac. In contrast to atropine, PACAP(6-38) did not inhibit Xenin-25 potentiation of the GIP-mediated increase in plasma insulin levels (FIG. 25C). Taken together, these results indicate that Xenin-25 indirectly potentiates GIP-mediated insulin release in vivo by increasing acetylcholine release from cholinergic parasympathetic neurons.

### Example 14

### Graded Glucose Infusion Trials in Humans

Subjects fasted overnight and were administered glucose intravenously. Such administration bypasses the endogenous incretin system. The glucose infusion started at time zero, and the glucose dose was increased every 40 minutes (1, 2, 3, 4, 6, and 8 mg/kg/min). A continuous intravenous infusion of a fixed dose of peptides (or peptide combination) was also started at time zero. Pharmacological doses of peptides were used, and each peptide (or combination) was infused on a separate occasion. Blood was collected every 10 min and plasma glucose and C-peptide were measured. The insulin secretion rate (ISR) was calculated by deconvolution of the C-peptide levels using means known in the art. The combination of xenin and GIP increased the ISR in subjects with impaired glucose tolerance compared to GIP administration alone. (FIGS. 26-27)

### Example 15

### Food Tolerance Trials in Humans

Subjects fasted overnight and ingested a liquid meal at time zero. This stimulates the release of endogenous incretins and other gut hormones (e.g. release of endogenous GIP). A continuous intravenous infusion of a fixed dose of xenin-25 or albumin alone was started at time zero. Blood was collected at the times indicated in FIGS. 28-30, and plasma glucose and C-peptide levels were measured. As in Example 15, the ISR was calculated by deconvolution of the C-peptide levels. The administration of xenin-25 decreased plasma glucose levels to near normal in individuals with impaired glucose tolerance (FIG. 28), and resulted in an earlier ISR in subjects with an impaired glucose tolerance (FIGS. 29 and 30).

### REFERENCES

1. Aiken, K. D., Kisslinger, J. A., and Roth, K. A. (1994) Developmental Dynamics 201, 636-70
2. Roth, K. A. and Gordon, J. I. (1990) Proceedings of the National Academy of Sciences 87, 6408-6412
3. Sjolund, K., Sanden, G., Hakanson, R., and Sundler, F. (1983) Gastroenterology 85, 1120-1130
4. Brand, S. J. and Schmidt, W. E. (1995) Gastrointestinal Hormones. In Yamada, T., editor. Textbook of Gastroenterology, JB Lippincott Company, Philadelphia
5. Walsh, J. H. (1994) Gastrointestinal Hormones. In Johnson, L. R., editor. Physiology of the Gastrointestinal Tract, Raven Press, New York
6. Miller, L. J. (1999) Gastrointestinal Hormones and Receptors. In Yamada, T., Alpers, D. H., Laine, L., Owyang, C., and Powell, D. W., editors. Textbook of Gastroenterology, Lippincott Williams & Wilkens, Philadelphia
7. Nakazato, M., Murakami, N., Date, Y., Kojima, M., Matsuo, H., Kangawa, K., and Matsukura, S. (2001) Nature 409, 194-198
8. Tschop, M., Smiley, D. L., and Heiman, M. L. (2000) Nature 407, 908-913
9. Asakawa, A., Inui, A., Kaga, T., Yuzuriha, H., Nagata, T., Ueno, N., Makino, S., Fujimiya, M., Niijima, A., Fujino, M. A., and Kasuga, M. (2001) Gastroenterology 120, 337-345
10. Schwartz, M. W., Woods, S. C., Porte, D. J., Seeley, R. J., and Baskin, D. G. (2000) Nature 404, 661-671
11. Batterham, R. L., Cowley, M. A., Small, C. J., Herzog, H., Cohen, M. A., Dakin, C. L., Wren, A. M., Brynes, A. E., Low, M. J., Ghatei, M. A., Cone, R. D., and Bloom, S. R. (2002) Nature 418, 650-654
12. Tseng, C., Jarboe, L. A., and Wolfe, M. M. (1994) American Journal of Physiology 266, G887-G891
13. Fehmann, H., Goke, R., and Goke, B. (1995) Endocrine Reviews 16, 390-410
14. Drucker, D. J. (1998) Diabetes 47, 159-169
15. Cataland, S., Crockett, S. E., Brown, J. C., and Mazzaferri, E. L. (1974) Journal of Clinical. Endocrinology & Metabolism 39, 223-228
16. Sykes, S., Morgan, L. M., English, J., and Marks, V. (1980) Journal of Endocrinology 85, 201-207
17. Wolfe, M. M., Zhao, K. B., Glazier, K. D., Jarboe, L. A., and Tseng, C. C. (2000) American. Journal of Physiology—Gastrointestinal & Liver Physiology 279, G561-G566
18. Thomas, F. B., Mazzaferri, E. L., Crockett, S. E., Mekhjian, H. S., Gruemer, H. D., and Cataland, S. (1976) Gastroenterology 70, 523-527
19. Falko, J. M., Crockett, S. E., Cataland, S., and Mazzaferri, E. L. (1975) Journal of Clinical. Endocrinology & Metabolism 41, 260-265
20. Bailey, C. J., Flatt, P. R., Kwasowski, P., Powell, C. J., and Marks, V. (1986) Acta Endocrinol (Copenh) 112, 224-229
21. Miyawaki, K., Yamada, Y., Ban, N., Ihara, Y., Tsukiyama, K., Zhou, H., Fujimoto, S., Oku, A., Tsuda, K., Toyokuni, S., Hiai, H., Mizunoya, W., Fushiki, T., Holst, J. J., Makino, M., Tashita, A., Kobara, Y., Tsubamoto, Y., Jinnouchi, T., Jomori, T., and Seino, Y. (2002) Nat Med 8, 738-742
22. Yip, R. G., Boylan, M. O., Kieffer, T. J., and Wolfe, M. M. (1998) Endocrinology 139, 4004-4007
23. Hauner, H., Glatting, G., Kaminska, D., and Pfeiffer, E. F. (1988) Ann Nutr Metab 32, 282-288
24. Starich, G. H., Bar, R. S., and Mazzaferri, E. L. (1985) Am J Physiol 249, E603-E607
25. Eckel, R. H., Fujimoto, W. Y., and Brunzell, J. D. (1979) Diabetes 28, 1141-1142
26. Knapper, J. M., Puddicombe, S. M., Morgan, L. M., Fletcher, J. M., and Marks, V. (1993) Biochem Soc Trans 21, 135S
27. Wasada, T., McCorkle, K., Harris, V., Kawai, K., Howard, B., and Unger, R. H. (1981) J Clin Invest 68, 1106-1107
28. Ebert, R., Nauck, M., and Creutzfeldt, W. (1991) Horm Metab Res 23, 517-521
29. Beck, B., Max, J. P., and Villaume, C. (1988) Int J Obes 12, 41-47
30. Oben, J., Morgan, L., Fletcher, J., and Marks, V. (1991) J Endocrinol 130, 267-272
31. Baba, A. S., Harper, J. M., and Buttery, P. J. (2000) Comp Biochem Physiol B Biochem Mol Biol 127, 173-182
32. Salera, M., Giacomoni, P., Pironi, L., Cornia, G., Capelli, M., Marini, A., Benfenati, F., Miglioli, M., and Barbara, L. (1982) J Clin Endocrinol Metab 55, 329-336
33. Morgan, L. M., Hampton, S. M., Tredger, J. A., Cramb, R., and Marks, V. (1988) Br J Nutr 59, 373-380
34. Elahi, D., Andersen, D. K., Muller, D. C., Tobin, J. D., Brown, J. C., and Andres, R. (1984) Diabetes 33, 950-957
35. Creutzfeldt, W., Ebert, R., Willms, B., Frerichs, H., and Brown, J. C. (1978) Diabetologia 14, 15-24
36. Ebert, R., Frerichs, H., and Creutzfeldt, W. (1979) Eur J Clin Invest 9, 129-135
37. Miyawaki, K., Yamada, Y., Yano, H., Niwa, H., Ban, N., Ihara, Y., Kubota, A., Fujimoto, S., Kajikawa, M., Kuroe, A., Tsuda, K., Hashimoto, H., Yamashita, T., Jomori, T., Tashiro, F., Miyazaki, J., and Seino, Y. (1999) Proceedings of the. National. Academy. of Sciences of the. United. States. of America 96, 14843-14847
38. Pamir, N., Lynn, F. C., Buchan, A. M., Ehses, J., Hinke, S. A., Pospisilik, J. A., Miyawaki, K., Yamada, Y., Seino, Y., McIntosh, C. H., and Pederson, R. A. (2003) Am J Physiol Endocrinol Metab 284, E931-E939
39. Ramshur, E. B., Rull, T. R., and Wice, B. M. (2002) Journal of Cellular Physiology 192, 339-350
40. Wang, S. Y., Chi, M. M., Li, L., Moley, K. H., and Wice, B. M. (2003) American Journal of Physiology—Endocrinology 284, E988-E1000
41. Wang, S. Y., Liu, J., Li, L., and Wice, B. M. (2004) Journal of Histochemistry and Cytochemistry 52, 53-63
42. Li, L. and Wice, B. M. (2005) Am. J. Physiol Endocrinol. Metab 288, E208-E215
43. Anlauf, M., Weihe, E., Hartschuh, W., Hamscher, G., and Feurle, G. E. (2000) J. Histochem. Cytochem. 48, 1617-1626
44. Silvestre, R. A., Rodriguez-Gallardo, J., Egido, E. M., Hernandez, R., and Marco, J. (2003) Regul. Pept. 115, 25-29
45. Feurle, G. E. (1998) Peptides 19, 609-615
46. Feurle, G. E., Hamscher, G., Kusiek, R., Meyer, H. E., and Metzger, J. W. (1992) J. Biol. Chem. 267, 22305-22309
47. Feurle, G. E., Heger, M., Niebergall-Roth, E., Teyssen, S., Fried, M., Eberle, C., Singer, M. V., and Hamscher, G. (1997) J. Pept. Res. 49, 324-330
48. Heuser, M., Kleiman, I., Popken, O., Nustede, R., and Post, S. (2002) Regul. Pept. 107, 23-27
49. Higashimoto, Y. and Liddle, R. A. (1993) Biochemical and Biophysical research communications 193, 182-190
50. Maxwell, F., Maxwell, I. H., and Glode, L. M. (1987) Mol Cell Biol 7, 1576-1579
51. Wice, B. M. and Gordon, J. I. (1998) Journal of Biological Chemistry 273, 25310-25319
52. Abbott, C. R., Small, C. J., Sajedi, A., Smith, K. L., Parkinson, J. R., Broadhead, L. L., Ghatei, M. A., and Bloom, S. R. (2006) Int. J. Obes. (Lond) 30, 288-292
53. Jandacek, R. J., Heubi, J. E., and Tso, P. (2004) Gastroenterology 127, 139-144
54. Persson, K., Gingerich, R. L., Nayak, S., Wada, K., Wada, E., and Ahren, B. (2000) Am. J. Physiol Endocrinol. Metab 279, E956-E962
55. Lu, W. J., Yang, Q., Sun, W., Woods, S. C., D'Alessio, D., and Tso, P. (2007) Am J Physiol Gastrointest. Liver Physiol 293, G963-G971
56. Cheung, A. T., Dayanandan, B., Lewis, J. T., Korbutt, G. S., Rajotte, R. V., Bryer-Ash, M., Boylan, M. O., Wolfe, M. M., and Kieffer, T. J. (2000) Science 2000 290, 1959-1962
57. Breitman, M. L., Bryce, D. M., Giddens, E., Clapoff, S., Goring, D., Tsui, L. C., Klintworth, G. K., and Bernstein, A. (1989) Development 106, 457-463
58. Breitman, M. L., Rombola, H., Maxwell, I. H., Klintworth, G. K., and Bernstein, A. (1990) Mol Cell Biol 10, 474-479
59. Palmiter, R. D., Behringer, R. R., Quaife, C. J., Maxwell, F., Maxwell, I. H., and Brinster, R. L. (1987) Cell 50, 435-443
60. Garabedian, E. M., Roberts, L. J., McNevin, M. S., and Gordon, J. I. (1997) J Biol Chem 272, 23729-23740
61. Itoh, H., Beck, P. L., Inoue, N., Xavier, R., and Podolsky, D. K. (1999) J Clin Invest 104, 1539-1547
62. Liddle, R. A. (1997) Annu. Rev. Physiol 59, 221-242
63. Theodorakis, M. J., Carlson, O., Michopoulos, S., Doyle, M. E., Juhaszova, M., Petraki, K., and Egan, J. M. (2006) Am J Physiol Endocrinol Metab 290, E550-E559
64. Mortensen, K., Christensen, L. L., Holst, J. J., and Orskov, C. (2003) Regul. Pept. 114, 189-196
65. Tseng, C., Boylan, M. O., Jarboe, L. A., Williams, E. K., Sunday, M. E., and Wolfe, M. M. (1995) Molecular and Cellular Endocrinology 115, 13-19
66. Zhou, L., Nian, M., Gu, J., and Irwin, D. M. (2006) Am. J. Physiol Regul. Integr. Comp Physiol 290, R634-R641
67. Hansotia, T., Maida, A., Flock, G., Yamada, Y., Tsukiyama, K., Seino, Y., and Drucker, D. J. (2007) J. Clin. Invest 117, 143-152
68. Preitner, F., Ibberson, M., Franklin, I., Binnert, C., Pende, M., Gjinovci, A., Hansotia, T., Drucker, D. J., Wollheim, C., Burcelin, R., and Thorens, B. (2004) J. Clin. Invest 113, 635-645
69. Hamscher, G., Meyer, H. E., and Feurle, G. E. (1996) Peptides 17, 889-893
70. Feurle, G. E., Ikonomu, S., Partoulas, G., Stoschus, B., and Hamscher, G. (2003) Regul. Pept. 111, 153-159
71. Nauck, M A, Heimesaat, M M, Orskov, C, Holst, J J, Ebert, R, Creutzfeldt, W: Preserved incretin activity of glucagon-like peptide 1 [7-36 amide] but not of synthetic human gastric inhibitory polypeptide in patients with type-2 diabetes mellitus. J Clin Invest 91:301-307, 1993
72. Elahi, D, oon-Dyke, M, Fukagawa, N K, Meneilly, G S, Sclater, A L, Minaker, K L, Habener, J F, Andersen, D K: The insulinotropic actions of glucose-dependent insulinotropic polypeptide (GIP) and glucagon-like peptide-1 (7-37) in normal and diabetic subjects. Regul Pept 51:63-74, 1994
73. Vilsboll, T, Krarup, T, Madsbad, S, Holst, J J: Defective amplification of the late phase insulin response to glucose by GIP in obese Type II diabetic patients. Diabetologia 45:1111-1119, 2002
74. Leiter, E H, Reifsnyder, P C: Differential levels of diabetogenic stress in two new mouse models of obesity and type 2 diabetes. Diabetes 53 Suppl 1:S4-11, 2004
75. Karlsson, O, Edlund, T, Moss, J B, Rutter, W J, Walker, M D: A mutational analysis of the insulin gene transcription control region: expression in beta cells is dependent on two related sequences within the enhancer. Proceedings of the National Academy of Sciences 84:8819-8823, 1987
76. Ahren, B: Autonomic regulation of islet hormone secretion—implications for health and disease. Diabetologia 43:393-410, 2000
77. Ahren, B, Holst, J J: The cephalic insulin response to meal ingestion in humans is dependent on both cholinergic and noncholinergic mechanisms and is important for postprandial glycemia. Diabetes 50:1030-1038, 2001
78. Ahren B: Neuropeptides and Insulin Secretion. In International Textbook of Diabetes Mellitus. Third ed. DeFronzo R A, Ferrannini E, Keen H, Zimmet P, Eds. Hoboken, New Jersey, John Wiley & Sons, Ltd, 2004, p. 153-163
79. Winzell, M S, Ahren, B: Role of VIP and PACAP in islet function. Peptides 28:1805-1813, 2007
80. McCullough, A J, Marshall, J B, Bingham, C P, Rice, B L, Manning, L D, Kalhan, S C: Carbachol modulates GIP-mediated insulin release from rat pancreatic lobules in vitro. Am J Physiol 248:E299-E303, 1985

What is claimed is:

1. A method for increasing GIP activity in a subject with impaired glucose tolerance, the method comprising administering to the subject a composition comprising an isolated compound selected from the group consisting of xenin-25, xenin-8, a xenin agonist, and a xenin antagonist, and at least one isolated compound selected from the group consisting of a glucose dependent insulinotropic polypeptide (GIP) protein, a homologue, analog, or ortholog thereof, a dipeptidyl peptidase IV (DPP IV) inhibitor, a cholinergic drug, an insulin sensitizer, and an insulin secretagogue.

2. The method of claim 1, wherein GIP activity is increased in the subject.

3. The method of claim 2, wherein the increased GIP activity induces increased insulin secretion.

4. A method for modulating insulin secretion in a subject with impaired glucose tolerance, the method comprising administering to the subject a composition comprising an isolated compound selected from the group consisting of xenin-25, xenin-8, a xenin agonist, and a xenin antagonist, and at least one isolated compound selected from the group consisting of a glucose dependent insulinotropic polypeptide (GIP) protein, a homologue, analog, or ortholog thereof, a dipeptidyl peptidase IV (DPP IV) inhibitor, a cholinergic drug, an insulin sensitizer, and an insulin secretagogue.

5. The method of claim 4, wherein insulin secretion is increased in the subject.

6. The method of claim 4, wherein the composition comprises xenin-25 and GIP.

7. The method of claim 4, wherein the composition comprises xenin-8 and GIP.
